(19) United States
(12) Patent Application Publication
(10) Pub. No.: US 2026/0240644 A1
(43) Pub. Date: Aug. 20, 2026

(54) BIOPOLYMER-BASED TYMPANOSTOMY TUBES WITH PROGRAMMABLE, TUNABLE, AND ON-DEMAND DEGRADATION

(71) Applicants: Trustees of Tufts College, Medford, MA (US); Massachusetts Eye and Ear Infirmary, Boston, MA (US)

(72) Inventors: David L. Kaplan, Concord, MA (US); Vincent P. Fitzpatrick, Somerville, MA (US); Glenn Leung, Fairhaven, MA (US); Riley Patten, Somerville, MA (US); Michael L. Lovett, Middleton, MA (US); Aaron Remenschneider, Boston, MA (US); Christopher Hartnick, Boston, MA (US)

(21) Appl. No.: 19/431,454

(22) Filed: Dec. 23, 2025

Related U.S. Application Data
(63) Continuation of application No. PCT/US2024/036397, filed on Jul. 1, 2024.
(60) Provisional application No. 63/511,548, filed on Jun. 30, 2023.

(51) Int. Cl. A61F 2/18; A61L 27/20; A61L 27/36

(52) U.S. Cl. CPC A61F 2/18; A61L 27/20; A61L 27/3641; A61F 2002/183; A61F 2230/0067; A61F 2230/0069; A61F 2240/001; A61F 2250/0059

(57) ABSTRACT

Disclosed herein is an ear tube including at least one biopolymer. The ear tube has at least one characteristic centrifugal property selected from the group consisting of: i) a uniform density across at least a portion of the ear tube, ii) at least a portion of the ear tube is optically nontransparent, iii) at least a portion of the ear tube is free of exterior layering, iv) at least a portion of the ear tube is free of interior layering, v) at least a portion of the ear tube is interior-void-free, vi) at least a portion of the ear tube is free of cylindrical extrusion gaps, and vii) at least a portion of the ear tube is free of layering.

### CROSS-REFERENCE TO RELATED APPLICATIONS

This application claims benefit of and is a continuation of International Patent Application No. PCT/US2024/036397 (Attorney Docket No. 2095.0615), filed Jul. 1, 2024, and entitled “BIOPOLYMER-BASED TYMPANOSTOMY TUBES WITH PROGRAMMABLE, TUNABLE, AND ON-DEMAND DEGRADATION,” International Pub. No. WO 2025/007128, which is hereby incorporated by reference in its entirety for all purposes.

International Patent Application No. PCT/US2024/036397 relates to, incorporates by reference for all purposes, and claims priority to U.S. Provisional Patent Application 63/511,548 (Attorney Docket No. 2095.0618), filed Jun. 30, 2023.

### GOVERNMENT FUNDING STATEMENT

This invention was made with government support under P41EB027062 awarded by the National Institutes of Health. The government has certain rights in the invention.

### INCORPORATION BY REFERENCE

This application incorporates by reference the following patents in their entirety for all purposes: U.S. Pat. No. 4,468,218, which issued on Aug. 28, 1984, and U.S. Pat. No. 11,229,726, which issued on Jan. 25, 2022. This application also incorporates by reference a related provisional application, U.S. Provisional Application No. 63/511,572, filed on Jun. 30, 2023 and entitled “CENTRIFUGALLY-MOLDED ARTICLES AND METHODS”, which provides a description of one particular method by which the ear tubes described herein can be made.

### BACKGROUND

Myringotomy and tympanostomy tube placement (M&T), is one of the most common pediatric surgeries in the United States, with nearly 700,000 children undergoing this procedure annually. These ventilation tubes (typical height and diameter of ~3 mm) are inserted following an incision (myringotomy) in the tympanic membrane (TM) as a means of bypassing the dysfunctional Eustachian tube and allowing drainage of the middle ear space.

Current commercially available tympanostomy tubes are made of synthetic, bioinert, and non-degradable materials such as fluoroplastic or silicone that are designed to self-extrude. Unfortunately, the extrusion process can be unpredictable, resulting in tubes that prematurely extrude or become stuck within the eardrum or ear canal and require surgical removal. Furthermore, tubes may become clogged or coated with biofilm resulting in non-ventilation of the middle ear or persistent otorrhea (ear drainage), in which case parents must administer antibiotic otic topical drops for up to a week. Given the sheer volume of M&T procedures, complications from tympanostomy tubes represent a significant burden on pediatric health—and innovative solutions in materials and designs that resolve complications from tympanostomy tubes would carry a large impact.

There are no current solutions that exist that address the issues described above. All existing commercial products are bioinert and non-resorbable. Furthermore, no existing product offers an “on-demand” degradation capability. Current approaches to issues with an implanted tympanostomy tube rely on surgical intervention under general anesthesia to remove the ear tube.

A need exists for new ear tubes that overcome one or more of the aforementioned shortcomings.

### SUMMARY

In some aspects, the techniques described herein relate to an ear tube including at least one biopolymer, the ear tube having at least one characteristic centrifugal property selected from the group consisting of: i) a uniform density across at least a portion of the ear tube; ii) at least a portion of the ear tube is optically nontransparent; iii) at least a portion of the ear tube is free of exterior layering; iv) at least a portion of the ear tube is free of interior layering; v) at least a portion of the ear tube is interior-void-free; vi) at least a portion of the ear tube is free of cylindrical extrusion gaps; and vii) at least a portion of the ear tube is free of layering.

In some aspects, the techniques described herein relate to an ear tube including at least one material assembled via an aqueous solution, the ear tube having at least one characteristic centrifugal property selected from the group consisting of: i) a uniform density across at least a portion of the ear tube; ii) at least a portion of the ear tube is optically nontransparent; iii) at least a portion of the ear tube is free of exterior layering; iv) at least a portion of the ear tube is free of interior layering; v) at least a portion of the ear tube is interior-void-free; vi) at least a portion of the ear tube is free of cylindrical extrusion gaps; and vii) at least a portion of the ear tube is free of layering.

In some aspects, the techniques described herein relate to an ear tube, including: a hollow cylindrical body having at least one flanged end, wherein the ear tube includes at least one biopolymer, the ear tube having at least one characteristic centrifugal property selected from the group consisting of: i) a uniform density across at least a portion of the ear tube; ii) at least a portion of the ear tube is optically nontransparent; iii) at least a portion of the ear tube is free of exterior layering; iv) at least a portion of the ear tube is free of interior layering; v) at least a portion of the ear tube is interior-void-free; vi) at least a portion of the ear tube is free of cylindrical extrusion gaps; and vii) at least a portion of the ear tube is free of layering.

In some aspects, the techniques described herein relate to an ear tube, including: a main tube, the main tube extending along a center axis defining a conduit for fluid communication, the main tube having a first end and a second end; and an outer flange extending radially away from the main tube on the first end, wherein the ear tube includes at least one biopolymer, the ear tube having at least one characteristic centrifugal property selected from the group consisting of: i) a uniform density across at least a portion of the ear tube; ii) at least a portion of the ear tube is optically nontransparent; iii) at least a portion of the ear tube is free of exterior layering; iv) at least a portion of the ear tube is free of interior layering; v) at least a portion of the ear tube is interior-void-free; vi) at least a portion of the ear tube is free of cylindrical extrusion gaps; and vii) at least a portion of the ear tube is free of layering.

In some aspects, the techniques described herein relate to an ear tube, including: a main tube, the main tube extending along a center axis defining a conduit for fluid communication, the main tube having a first end and a second end; an outer flange extending radially away from the main tube on a first end; an inner flange extending radially away from the main tube on a second end; and a frustoconical portion of the main tube extending longitudinally from at least one of the first end or the second end, the frustoconical portion extending beyond a width of the flange on the at least one of the first end or the second end, wherein the ear tube includes at least one biopolymer, the ear tube having at least one characteristic centrifugal property selected from the group consisting of: i) a uniform density across at least a portion of the ear tube; ii) at least a portion of the ear tube is optically nontransparent; iii) at least a portion of the ear tube is free of exterior layering; iv) at least a portion of the ear tube is free of interior layering; v) at least a portion of the ear tube is interior-void-free; vi) at least a portion of the ear tube is free of cylindrical extrusion gaps; and vii) at least a portion of the ear tube is free of layering.

In some aspects, the techniques described herein relate to a method of forming an ear tube, including: depositing at least one aqueous biopolymer solution into an ear tube mold; subjecting the ear tube mold and its contents to a first centrifugation; depositing a polymerization bath into the ear tube mold and subjecting the ear tube mold and its contents to a second centrifugation to polymerize the aqueous biopolymer solution into a shape of an ear tube; and drying the ear tube.

In some aspects, the techniques described herein relate to a method of degrading an ear tube, including: exposing an ear tube to a solvent, wherein the ear tube includes at least one biopolymer having at least one characteristic centrifugal property selected from the group consisting of: i) a uniform density across at least a portion of the ear tube; ii) at least a portion of the ear tube is optically nontransparent; iii) at least a portion of the ear tube is free of exterior layering; iv) at least a portion of the ear tube is free of interior layering; v) at least a portion of the ear tube is interior-void-free; vi) at least a portion of the ear tube is free of cylindrical extrusion gaps; and vii) at least a portion of the ear tube is free of layering.

These and other systems, methods, objects, features, and advantages of the present disclosure will be apparent to those skilled in the art from the following detailed description of the preferred embodiment and the drawings.

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

The patent or application file contains at least one drawing executed in color. Copies of this patent or patent application publication with color drawing(s) will be provided by the Office upon request and payment of the necessary fee.

FIG. 1 depicts ear tube mold designs.

FIG. 2 depicts example ear tubes.

FIG. 3 depicts multimaterial designs for the ear tubes.

FIG. 4 depicts example ear tubes.

FIG. 5 depicts glucose being used in enzymatically mediated degradation of chitosan by hydrogen peroxide.

FIG. 6 depicts results of a Kirby-Bauer disc diffusion assay on silk and chitosan discs, coated or not with Ciprodex. n=6. Measurements were taken after 24 hours of culture with Staphylococcus aureus, at 37° C. Statistical analysis performed by paired t-test on control and dip coated groups. P-values shown on the graph.

FIG. 7A and FIG. 7B depict: (A,B) Weight loss from enzymatic degradation of silk films (FIG. 7A) and hydrogels (FIG. 7B), prepared from a 3% (w/v) silk solution and immersed in proteinase K, protease XIV, α-chymotrypsin, collagenase, MMP-1, MMP-2, or deionized water (control). Remaining mass of material was measured after each time point and compared against the control. Values are the average±standard deviation of N=5. Adapted from Brown et al. 2015. (FIG. 7C) Changes in the molecular weight of 0.5 g/dL shrimp chitosan (DD) 84%) solution after reacting with different initial concentrations of H₂O₂ at 80° C. for different times.

FIG. 8 depicts a collar ear tube design.

FIG. 9 depicts a degradable inner flange.

FIG. 10A depicts a Side view of chitosan ear tube (left) vs. silicone ear tube (right); FIG. 10B depicts a top view of chitosan ear tube (left) vs. silicone ear tube (right). Scale bar 1 mm.

FIG. 11A and FIG. 11B depict a timeline of an initial study design.

FIG. 12 depicts InCrowd Survey of ENT surgeon preferences (n=10).

FIG. 13 depicts Hematoxylin- and Eosin-stained slides of explanted chitosan discs implanted subcutaneously in the back of rats.

FIG. 14 depicts Modified Study Groups and Timeline.

FIG. 15 depicts otoscope pictures taken at different timepoints after implantation.

FIG. 16 depicts: Left: Representative images of ear tubes 3 weeks after implantation, showing patency; Right: representative image of one wax-blocked ear tube, 3 weeks after implantation.

FIG. 17A depicts: a representative tympanogram-no perforation. Form of curve indicates canal & middle ear not communicating (e.g., healthy membrane, healed membrane after dissolution, healed membrane after tube extrusion, or blocked tube). FIG. 17B depicts a representative tympanogram-open due to tube. Form of curve indicates canal & middle ear communicating (e.g., patent tube).

FIG. 18A depicts a Side view of unused chitosan ear tube (left) vs. degraded chitosan ear tube covered in wax (right) Scale bar 1 mm; FIG. 18B is a front view of unused chitosan ear tube (left) vs. degraded chitosan ear tube covered in wax (right). Scale bar 1 mm. FIG. 18C is a diagram of ear tube positioning, showing middle ear and ear canal (fromlancastergeneralhealth.org); FIG. 18D depicts extruded silicone tube (from reddit.com).

FIG. 19A-19E depict prototype ear tube undergoing benchtop degradation simulation shows 5% hydrogen peroxide degrades tube sooner. It was inserted into a slit within a nitrile sheet under tension. For 3 days, 3% (top row) or 5% (bottom row) hydrogen peroxide was deposited topically onto the apparatus and allowed to dwell for 10 minutes. (FIG. 19A) Day 1 before treatment. (FIG. 19B) Day 1 after treatment. (FIG. 19C) Day 2 before treatment. (FIG. 19D) Day 2 after treatment. (FIG. 19E) Day 3 before treatment. (FIG. 19F) Day 3 after treatment. Scale bar: 1 mm

FIGS. 20A and 20B depicts three weeks after implantation (no hydrogen peroxide treatment): Representative DPOAE curve (t=3 weeks) and tables (before implantation and t=3 weeks), representative tympanogram and otoscope image (t=3 weeks).

FIGS. 21A and 21B depict after degradation with hydrogen peroxide: Representative DPOAE curve (t=4 weeks) and tables (before implantation and t=4 weeks), representative tympanogram and otoscope image (t=4 weeks).

FIG. 22 depicts a summary of the main in vivo findings.

FIG. 23 depicts results of the PrestoBlue assay using L929 fibroblasts, after 6 days of culture.

FIG. 24 depicts stainless steel molds for ear tube manufacturing.

FIG. 25 depicts mechanical properties of chitosan ear tubes vs commercial ear tubes. Left: yield force; Right: Young's modulus.

FIG. 26 depicts release kinetics of ciprofloxacin and dexamethasone from loaded chitosan constructs, in PBS at 37° C.

FIG. 27 depicts results of the Kirby Bauer disc diffusion assay.

### DETAILED DESCRIPTION

Before the present invention is described in further detail, it is to be understood that the invention is not limited to the particular embodiments described. It is also understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

Specific structures, devices, and methods relating to surface patterning are disclosed. It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

As used herein, “free of cylindrical extrusion gaps” refers to a lack of observable gaps within an article that are characteristic of a three-dimensional printing process that utilizes extruded cylinders of polymers.

As used herein, “free of layering” refers to a lack of observable layer structures within an article. Layering can be observed by imaging (e.g., scanning electron microscopy), spectroscopy (e.g., FTIR or fluorescence spectroscopy), or other analytical method (e.g., differential scanning calorimetry). Examples of techniques that do exhibit layering in their resulting products (i.e., these do not produce products that are free of layering, to the best of our understanding) are dip-coating (the separate coating layers can be distinctly identified) or three-dimensional printing (the printing process is typically a layered process that can be identified).

As used herein, “free of exterior layering” refers to an article being free of layering as observed from an exterior of the article.

As used herein, “free of interior layering” refers to an article being free of layering as observed internally within the article. In most cases, the absence of interior layering can be observed by cross-sectioning the article and assessing the newly-exposed face for evidence of layering.

As used herein, “interior-void-free” refers to an article that lacks interior voids having a largest physical dimension of between 50 μm and 1 mm or between 50 μm and 200 μm. It should be appreciated that certain porogen approaches could be used to generate an article that has intentionally-placed pores amidst a solid framework that itself is interior-void-free. In this instance, an interior-void-free article could be adapted to possess pores that fall within the size definitions here, but those pores would readily be identified by a skilled artisan as different and distinct from voids that are unintentionally formed in the interior of an article.

As used here, “optically nontransparent” refers to a material having a visible light attenuation for a predetermined visible wavelength (or range of wavelengths) of 50% or greater, including but not limited to, 60% or greater, 70% or greater, 80% or greater, 90% or greater, or 95% or greater.

As used herein, “random sampling” refers to any random or pseudo-random selection of sampling locations from an article. Some techniques may be as close to truly random as possible, while some may be pseudo-random. It is contemplated that the random nature of the sampling may be randomized on an article-by-article basis or may be randomized once for a whole series of articles. As one specific example, a script can be crafted for imaging software to randomly select sampling locations by using a grid array and a random number generator. In some cases, random sampling involves more than one random selection (e.g., a random selection of which cross-section(s) to take of an article, followed by a random selection of which location(s) on the newly-exposed faces to analyze).

As used herein, “representative sampling” refers to a sampling method for assessing an article for a given property that provide a skilled artisan with at least 90%, at least 95%, or at least 99% confidence that the sampling selection provides an accurate measurement of the given property throughout a predetermined volume (e.g., the entire article or a specific region of the article). Naturally, increasing the number of samples taken will increase the confidence. The locations of the samples are chosen or randomly selected to ensure adequate coverage for the above-referenced confidence level. In some cases, representative sampling involves more than one representative selection (e.g., a representative selection of which cross-section(s) to take of an article, followed by a representative selection of which location(s) on the newly-exposed faces to analyze).

As used herein, a “uniform” property is a property that varies by less than 10%, less than 5%, less than 1%, or less than 0.1% across the entirety of an article, as shown by a random sampling, a representative sampling, or another sampling technique that has been shown to provide statistically valid sampling.

As used herein, “silk fibroin” refers to silk fibroin protein whether produced by silkworm, spider, or other insect, or otherwise generated (Lucas et al., Adv. Protein Chem., 13:107-242 (1958)). Any type of silk fibroin can be used in different embodiments described herein. Silk fibroin produced by silkworms, such as Bombyx mori, is the most common and represents an earth-friendly, renewable resource. For instance, silk fibroin used in a silk film may be attained by extracting sericin from the cocoons of B. mori. Organic silkworm cocoons are also commercially available. There are many different silks, however, including spider silk (e.g., obtained from Nephila clavipes), transgenic silks, genetically engineered silks, such as silks from bacteria, yeast, mammalian cells, transgenic animals, or transgenic plants, and variants thereof, that can be used. See, e.g., WO 97/08315 and U.S. Pat. No. 5,245,012, each of which is incorporated herein by reference in their entireties.

Disclosed herein are new tympanostomy tubes (ear tubes) made of biopolymers (e.g., silk, chitosan, and combinations thereof). These ear tubes present several advantages over existing solutions. In one example, the disclosed ear tubes are made of natural, non-synthetic biopolymers, with lesser risk of biocompatibility issues. In one example, the disclosed ear tubes can be functionalized with bioactive molecules like antibiotics or anti-inflammatory drugs in order to present better outcomes for the patient, and to minimize costs of the separate administration of drugs (e.g., Ciprodex, which is commonly administered in the form of drops placed in the ear canal after tube implantation). In one example, the disclosed ear tubes' degradation can be controlled. This can take place in several forms: tuning of degradation over a specific timeframe (for example by loading enzymes into the ear tube), being able to specifically start the degradation process on demand, by the healthcare provider. This latter feature is extremely desirable for clinicians and patients, because it allows ear tube removal when necessary, without the need for general anesthesia or a trip to the operating room. It also allows removal of an ear tube that might get stuck in the ear tube, an event which would also typically require an operation with general anesthesia. As such, this can greatly improve patient care, and significantly decrease costs. Thus, certain benefits include non-surgical removal, reduced scar tissue formation (a proxy for hearing preservation), and the overall reduction in medical treatment visits and use of general anesthesia, leading to improved health equity.

Chitosan, which can be used to make the disclosed ear tubes, is a naturally occurring polysaccharide biopolymer, and its derivatives are non-toxic, biocompatible, and biodegradable with certain important biological properties, such as intrinsic antimicrobial properties, mucoadhesion, and permeability enhancing properties, which may be imparted to the ear tubes comprising chitosan or a derivative thereof. Other biological activities may include antifungal, antitumor, anticancer, anti-diabetic, wound healing, and antioxidant activities. Depending on the degree of deacetylation, the degree of modification, and/or the molecular weight (MW) of chitosan, certain properties (e.g., bioactivity, biocompatibility, and biodegradability) may be present, absent, enhanced, or less prominent. Example curing solutions to be used with chitosan include sodium hydroxide, a mixture of sodium hydroxide and ethanol, ethanol, urea (carbamide), carbonic acid, or sodium tripolyphosphate.

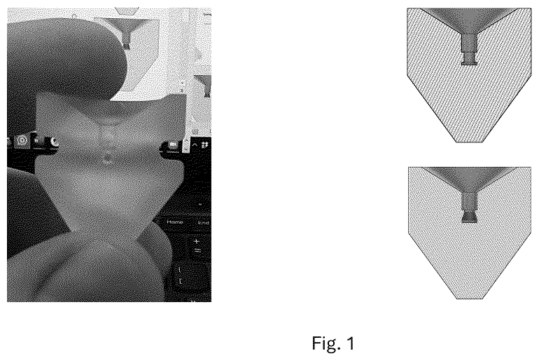

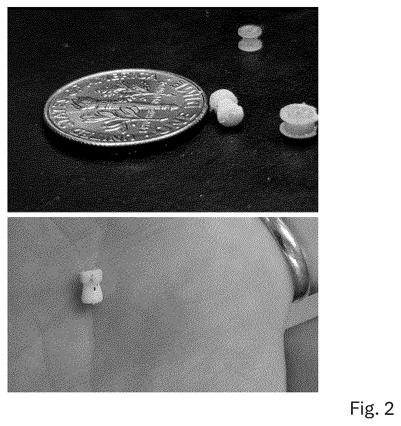

Disclosed herein is a novel bioengineered tympanostomy tube for the treatment of chronic to improve middle ear ventilation in children who suffer from ear infections that do not resolve on their own or with standard treatments (e.g., antibiotics), or for children predisposed to recurring ear infections. This procedure, known as myringotomy and tympanostomy tube placement (M&T), is one of the most common pediatric surgeries in the United States, with nearly 700,000 children undergoing this procedure annually. During the procedure, an incision (myringotomy) is made in the tympanic membrane, the fluid in the middle ear is suctioned, and a ventilation tube (typical height and diameter of ~3 mm) is inserted to promote ventilation of the middle ear. Disclosed herein is a unique bioengineered biopolymer-based tympanostomy tube that can locally elute antibiotics and anti-inflammatory agents to prevent post-operative infections while also degrading on-demand without surgical intervention. FIG. 1 depicts a mold for the ear tube (left, physical mold; right, two different designs) and the resulting ear tubes (FIG. 2) are shown.

The use of biopolymers, centrifugal molding and a mild aqueous-based method of polymerization allows the loading of bioactive molecules like antibiotics or anti-inflammatory agents, which will improve patient outcomes. These molecules can be loaded directly into the biopolymer solution prior to casting. Alternatively, these molecules can be loaded post molding using approaches like dipcoating.

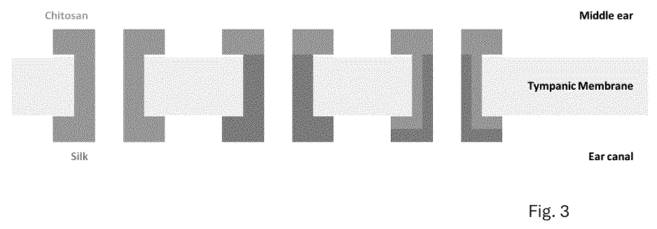

Disclosed herein, and referring to FIG. 3, are multimaterial designs for the ear tubes. In addition to the simple chitosan ear tube, degradation of the inner flange is disclosed, to allow extrusion of the ear tube only towards the ear canal, where it can be removed.

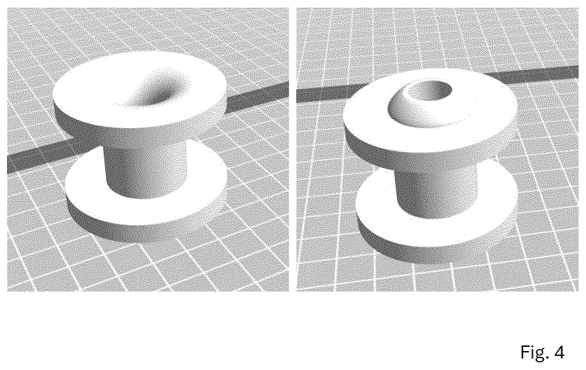

Using a molding approach, new designs of the outer flanges or the internal topography of the tube are possible, to adjust surface tension, to improve drainage and ventilation of the middle ear (see FIG. 4). Alternative approaches of manufacturing include thermal molding followed by machining.

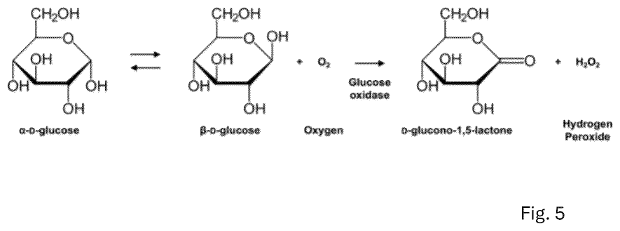

Alternative approaches of tuning degradation include loading with enzymes. For example, loading silk tubes with protease XIV enables tunable degradation. In another example of tunable degradation, chitosan or silk/chitosan composite may be loaded with encapsulated glucose oxidase. In another example, a glucose solution may be used to induce the enzymatically mediated degradation of the chitosan by hydrogen peroxide (see FIG. 5).

Disclosed herein are ear tubes including at least one biopolymer. Example ear tubes have at least one characteristic centrifugal property selected from the group consisting of: i) a uniform density across at least a portion of the ear tube, ii) at least a portion of the ear tube is optically nontransparent, iii) at least a portion of the ear tube is free of exterior layering, iv) at least a portion of the ear tube is free of interior layering, v) at least a portion of the ear tube is interior-void-free, vi) at least a portion of the ear tube is free of cylindrical extrusion gaps, and vii) at least a portion of the ear tube is free of layering.

In an example ear tube, at least one of an outer flange or an inner flange of the ear tube is angled. In the example, at least one of the outer flange or inner flange defines a plane which is inclined at an angle with respect to a plane disposed perpendicular to an axis of a main tube of the ear tube. The angle is between 15 degrees and 45 degrees, including but not limited to, at least 15 degrees, at least 30 degrees, or at least 45 degrees. In an example, at least one of the outer flange or inner flange includes an insertion protuberance.

In an example ear tube, an outer flange and an inner flange of the ear tube are at least one of same or different with respect to at least one of a thickness, a relative angle, a shape, an orientation, or a geometry. In an example ear tube, the ear tube has a uniform diameter throughout its length. In an example ear tube, the ear tube has a non-uniform diameter along its length. In an example ear tube, the ear tube tapers along its length. In an example ear tube, the ear tube has a uniform density throughout.

In an example ear tube, the ear tube includes a cross-sectional profile that is uniform throughout the ear tube. The uniform cross-sectional profile is determined by at least one of a visual inspection, scanning electron microscopy, fluorescence microscopy, DSC, TEM, XRD, EPMA, or FTIR. The cross-sectional profile relates to at least one of a density, an absence of voids, a consistent pattern, or a chemical profile.

In an example ear tube, a main tube of the ear tube has an axial length at least several times its diameter.

In an example ear tube, a main tube of the ear tube has an inner diameter of between 0.1 and 2.9 mm. In an example ear tube, a main tube of the ear tube has an inner diameter of between 0.1 and 2.5 mm. In an example ear tube, a main tube of the ear tube has an inner diameter of between 0.1 and 2 mm. In an example ear tube, a main tube of the ear tube has an inner diameter of between 0.1 and 1.5 mm. In an example ear tube, a main tube of the ear tube has an inner diameter of between 0.1 and 1 mm. In an example ear tube, a main tube of the ear tube has an inner diameter of between 1.0 and 3. In an example ear tube, a main tube of the ear tube has an inner diameter of, between 1.0 and 2 mm. In an example ear tube, a main tube of the ear tube has an inner diameter of between 1.0 and 1.5 mm. In an example ear tube, a main tube of the ear tube has an inner diameter of at least 0.1 mm. In an example ear tube, a main tube of the ear tube has an inner diameter of at least 0.5 mm. In an example ear tube, a main tube of the ear tube has an inner diameter of at least 0.6 mm. In an example ear tube, a main tube of the ear tube has an inner diameter of at least 0.7 mm. In an example ear tube, a main tube of the ear tube has an inner diameter of at least 0.8 mm. In an example ear tube, a main tube of the ear tube has an inner diameter of at least 0.9 mm. In an example ear tube, a main tube of the ear tube has an inner diameter of at least 1.0 mm. In an example ear tube, a main tube of the ear tube has an inner diameter of at least 1.1 mm. In an example ear tube, a main tube of the ear tube has an inner diameter of at least 1.2 mm. In an example ear tube, a main tube of the ear tube has an inner diameter of at least 1.3 mm. In an example ear tube, a main tube of the ear tube has an inner diameter of at least 1.4 mm. In an example ear tube, a main tube of the ear tube has an inner diameter of at least 1.5 mm. In an example ear tube, a main tube of the ear tube has an inner diameter of at least 2.0 mm. In an example ear tube, a main tube of the ear tube has an inner diameter of at least 2.5 mm. In an example ear tube, a main tube of the ear tube has an inner diameter of at least 3.0 mm. In an example ear tube, a main tube of the ear tube has an inner diameter of at least 3.8 mm.

In an example ear tube, a main tube of the ear tube has an outer diameter of between 0.1 and 2.9 mm. In an example ear tube, a main tube of the ear tube has an outer diameter of between 0.1 and 2.5 mm. In an example ear tube, a main tube of the ear tube has an outer diameter of between 0.1 and 2 mm. In an example ear tube, a main tube of the ear tube has an outer diameter of between 0.1 and 1.5 mm. In an example ear tube, a main tube of the ear tube has an outer diameter of between 0.1 and 1 mm. In an example ear tube, a main tube of the ear tube has an outer diameter of between 1.0 and 3 mm. In an example ear tube, a main tube of the ear tube has an outer diameter of between 1.0 and 2 mm. In an example ear tube, a main tube of the ear tube has an outer diameter of between 1.0 and 1.5 mm. In an example ear tube, a main tube of the ear tube has an outer diameter of at least 0.1 mm. In an example ear tube, a main tube of the ear tube has an outer diameter of at least 0.5 mm. In an example ear tube, a main tube of the ear tube has an outer diameter of at least 0.6 mm. In an example ear tube, a main tube of the ear tube has an outer diameter of at least 0.7 mm. In an example ear tube, a main tube of the ear tube has an outer diameter of at least 0.8 mm. In an example ear tube, a main tube of the ear tube has an outer diameter of at least 0.9 mm. In an example ear tube, a main tube of the ear tube has an outer diameter of at least 1.0 mm. In an example ear tube, a main tube of the ear tube has an outer diameter of at least 1.1 mm. In an example ear tube, a main tube of the ear tube has an outer diameter of at least 1.2 mm. In an example ear tube, a main tube of the ear tube has an outer diameter of at least 1.3 mm. In an example ear tube, a main tube of the ear tube has an outer diameter of at least 1.4 mm. In an example ear tube, a main tube of the ear tube has an outer diameter of at least 1.5 mm. In an example ear tube, a main tube of the ear tube has an outer diameter of at least 2.0 mm. In an example ear tube, a main tube of the ear tube has an outer diameter of at least 2.5 mm. In an example ear tube, a main tube of the ear tube has an outer diameter of at least 3.0 mm. In an example ear tube, a main tube of the ear tube has an outer diameter of at least 3.8 mm.

In an example ear tube, the ear tube has an inner flange. The inner flange diameter is between 1.0 mm and 10.0 mm. The inner flange diameter is between 2.0 mm and 9.0 mm. The inner flange diameter is between 2.0 mm and 7.0 mm. The inner flange diameter is between 3.0 mm and 6.0 mm. The inner flange diameter is at least 1 mm. The inner flange diameter is at least 1.5 mm. The inner flange diameter is at least 2.0 mm. The inner flange diameter is at least 3.0 mm. The inner flange diameter is at least 4.0 mm. The inner flange diameter is at least 5.0 mm. The inner flange diameter is at least 6.0 mm. The inner flange diameter is at least 7.0 mm. The inner flange diameter is at least 8.0 mm. The inner flange diameter is at least 9.0 mm. The inner flange diameter is at least 10.0 mm.

In an example ear tube, the ear tube has an outer flange. The outer flange diameter is between 1.0 mm and 10.0 mm. The outer flange diameter is between 2.0 mm and 9.0 mm. The outer flange diameter is between 2.0 mm and 7.0 mm. The outer flange diameter is between 3.0 mm and 6.0 mm. The outer flange diameter is at least 1 mm. The outer flange diameter is at least 1.5 mm. The outer flange diameter is at least 2.0 mm. The outer flange diameter is at least 3.0 mm. The outer flange diameter is at least 4.0 mm. The outer flange diameter is at least 5.0 mm. The outer flange diameter is at least 6.0 mm. The outer flange diameter is at least 7.0 mm. The outer flange diameter is at least 8.0 mm. The outer flange diameter is at least 9.0 mm. The outer flange diameter is at least 10.0 mm.

In an example ear tube, a wall of a main tube of the ear tube has a thickness of between 0.1 mm and 0.5 mm. In an example ear tube, a wall of a main tube of the ear tube has a thickness of between 0.1 mm and 0.4 mm. In an example ear tube, a wall of a main tube of the ear tube has a thickness of between 0.3 mm and 0.5 mm. In an example ear tube, a wall of a main tube of the ear tube has a thickness of at least 0.1 mm. In an example ear tube, a wall of a main tube of the ear tube has a thickness of at least 0.2 mm. In an example ear tube, a wall of a main tube of the ear tube has a thickness of at least 0.3 mm. In an example ear tube, a wall of a main tube of the ear tube has a thickness of at least 0.4 mm. In an example ear tube, a wall of a main tube of the ear tube has a thickness of at least 0.5 mm.

In an example ear tube, a distance between a top surface of an inner flange of the ear tube and a bottom surface of an outer flange of the ear tube is between 0.05 mm and 12.0 mm. In an example ear tube, a distance between a top surface of an inner flange of the ear tube and a bottom surface of an outer flange of the ear tube is between 0.1 mm and 7.5 mm. In an example ear tube, a distance between a top surface of an inner flange of the ear tube and a bottom surface of an outer flange of the ear tube is between 1 mm and 7.5 mm. In an example ear tube, a distance between a top surface of an inner flange of the ear tube and a bottom surface of an outer flange of the ear tube is between 2.0 mm and 5.0 mm. In an example ear tube, a distance between a top surface of an inner flange of the ear tube and a bottom surface of an outer flange of the ear tube is at least 0.05 mm. In an example ear tube, a distance between a top surface of an inner flange of the ear tube and a bottom surface of an outer flange of the ear tube is at least 0.1 mm. In an example ear tube, a distance between a top surface of an inner flange of the ear tube and a bottom surface of an outer flange of the ear tube is at least 0.5 mm. In an example ear tube, a distance between a top surface of an inner flange of the ear tube and a bottom surface of an outer flange of the ear tube is at least 1.0 mm. In an example ear tube, a distance between a top surface of an inner flange of the ear tube and a bottom surface of an outer flange of the ear tube is at least 2.0 mm. In an example ear tube, a distance between a top surface of an inner flange of the ear tube and a bottom surface of an outer flange of the ear tube is at least 3.0 mm. In an example ear tube, a distance between a top surface of an inner flange of the ear tube and a bottom surface of an outer flange of the ear tube is at least 4.0 mm. In an example ear tube, a distance between a top surface of an inner flange of the ear tube and a bottom surface of an outer flange of the ear tube is at least 5.0 mm. In an example ear tube, a distance between a top surface of an inner flange of the ear tube and a bottom surface of an outer flange of the ear tube is at least 7.5 mm. In an example ear tube, a distance between a top surface of an inner flange of the ear tube and a bottom surface of an outer flange of the ear tube is at least 10.0 mm. In an example ear tube, a distance between a top surface of an inner flange of the ear tube and a bottom surface of an outer flange of the ear tube is at least 12.0 mm.

In an example ear tube, a total length of the ear tube is between 0.5 and 3 mm, between 0.5 and 2.5 mm, between 1 and 2.5 mm, between 1 and 2 mm, between 1 and 1.5 mm, between 2.0 mm and 5.0 mm, or between 3.0 mm and 8.0 mm, including but not limited to, at least 1.5 mm, at least 2.0 mm. at least 3.0 mm, at least 4.0 mm, at least 5.0 mm, at least 7.5 mm, or at least 8.0 mm.

In an example ear tube, an outer flange and an inner flange of the ear tube are formed from a same biopolymer. In an example ear tube, an outer flange and an inner flange of the ear tube are formed from different biopolymers.

In an example ear tube, an outer flange and an inner flange of the ear tube are formed from at least two biopolymers, each of the at least two biopolymers defining a thickness of the flange.

In an example ear tube, a main tube of the ear tube is formed from a single biopolymer. In an example ear tube, a main tube of the ear tube is formed from a plurality of biopolymers, wherein a first biopolymer of the plurality of biopolymers forms an inner surface of the main tube and a second biopolymer of the plurality of biopolymers is disposed circumferentially around the first biopolymer and forms an outer surface of the main tube.

In an example ear tube, the ear tube comprises a first biopolymer forming at least a portion of a main tube of the ear tube and at least a portion of at least one flange, and a second biopolymer forming at least a portion of the main tube of the ear tube and at least a portion of at least one flange.

In an example ear tube, an inner flange of the ear tube comprises chitosan and a main tube of the ear tube and outer flange comprise silk.

In an example ear tube, the ear tube includes at least one biopolymer. The at least one biopolymer is silk fibroin, or at least one of silk fibroin or chitosan polysaccharide.

In an example ear tube, the ear tube includes silk fibroin and chitosan polysaccharide. In an example ear tube, the ear tube includes silk fibroin and alginate.

In an example ear tube, the ear tube includes at least one biopolymer, the at least one biopolymer is an aqueous silk solution, an aqueous alginate solution, an aqueous fibrinogen solution, an aqueous chitosan solution, or an aqueous collagen solution.

In an example ear tube, the ear tube is formed via centrifugal molding, via thermal molding and machining, or via centrifugal molding, thermal molding, and machining.

In an example ear tube, the ear tube includes at least one biopolymer, the at least one biopolymer is assembled via an aqueous-based polymerization.

In an example ear tube, the ear tube comprises at least one additive. In the example, the at least one additive is at least one of a bioactive molecule, a hydrophilic plasticizer, a hydrophobic plasticizer, a pigment, or a dye. The bioactive molecule is an active pharmaceutical ingredient, an antibiotic, an anti-inflammatory agent, a steroid, a growth factor, or an enzyme. The enzyme is at least one of a proteolytic enzyme, a serine protease, protease XIV, proteinase K. alpha-chymotrypsin, collagenase, MMP-1, MMP-2, or glucose oxidase. In the example, the at least one additive is dip-coated onto the ear tube. The dip-coated ear tube is subjected to water annealing. In the example, the additive is dissolved in a biopolymer solution before dip-coating. In the example, the ear tube is impregnated with the at least one additive.

In an example ear tube, the ear tube is designed to degrade on demand. In the example, degradation commences when the ear tube is exposed to a solvent. The solvent is at least one of a glucose solution, hydrogen peroxide, carbamide peroxide, an alcohol, a calcium chelator, or EDTA.

In an example ear tube, the ear tube is a tympanostomy tube.

Disclosed herein is an ear tube including at least one material assembled via an aqueous solution, the ear tube having at least one characteristic centrifugal property selected from the group consisting of: i) a uniform density across at least a portion of the ear tube, ii) at least a portion of the ear tube is optically nontransparent, iii) at least a portion of the ear tube is free of exterior layering, iv) at least a portion of the ear tube is free of interior layering, v) at least a portion of the ear tube is interior-void-free, vi) at least a portion of the ear tube is free of cylindrical extrusion gaps, and vii) at least a portion of the ear tube is free of layering.

In an example ear tube, at least one of an outer flange or an inner flange is angled. In the example, the at least one of the outer flange or inner flange defines a plane which is inclined at an angle with respect to a plane disposed perpendicular to an axis of a main tube. The angle is between 15 degrees and 45 degrees, including but not limited to, at least 15 degrees, at least 30 degrees, or at least 45 degrees. In the example, at least one of the outer flange or inner flange comprises an insertion protuberance.

In an example ear tube, an outer flange and an inner flange of the ear tube are at least one of same or different with respect to at least one of a thickness, a relative angle, a shape, an orientation, or a geometry.

In an example ear tube, the ear tube has a uniform diameter throughout its length. In an example ear tube, the ear tube has a non-uniform diameter along its length. In an example ear tube, the ear tube tapers along its length. In an example ear tube, the ear tube has a uniform density throughout.

In an example ear tube, the ear tube includes a cross-sectional profile that is uniform throughout the ear tube. In the example, the uniform cross-sectional profile is determined by at least one of a visual inspection, scanning electron microscopy, fluorescence microscopy, DSC, TEM, XRD, EPMA, or FTIR. In the example, the cross-sectional profile relates to at least one of a density, an absence of voids, a consistent pattern, or a chemical profile.

In an example ear tube, a main tube of the ear tube has an axial length at least several times its diameter.

In an example ear tube, a main tube of the ear tube has an inner diameter of between 0.1 and 2.9 mm. In an example ear tube, a main tube of the ear tube has an inner diameter of between 0.1 and 2.5 mm. In an example ear tube, a main tube of the ear tube has an inner diameter of between 0.1 and 2 mm. In an example ear tube, a main tube of the ear tube has an inner diameter of between 0.1 and 1.5 mm. In an example ear tube, a main tube of the ear tube has an inner diameter of between 0.1 and 1 mm. In an example ear tube, a main tube of the ear tube has an inner diameter of between 1.0 and 3 mm. In an example ear tube, a main tube of the ear tube has an inner diameter of between 1.0 and 2 mm. In an example ear tube, a main tube of the ear tube has an inner diameter of between 1.0 and 1.5 mm. In an example ear tube, a main tube of the ear tube has an inner diameter of at least 0.1 mm. In an example ear tube, a main tube of the ear tube has an inner diameter of at least 0.5 mm. In an example ear tube, a main tube of the ear tube has an inner diameter of at least 0.6 mm. In an example ear tube, a main tube of the ear tube has an inner diameter of at least 0.7 mm. In an example ear tube, a main tube of the ear tube has an inner diameter of at least 0.8 mm. In an example ear tube, a main tube of the ear tube has an inner diameter of at least 0.9 mm. In an example ear tube, a main tube of the ear tube has an inner diameter of at least 1.0 mm. In an example ear tube, a main tube of the ear tube has an inner diameter of at least 1.1 mm. In an example ear tube, a main tube of the ear tube has an inner diameter of at least 1.2 mm. In an example ear tube, a main tube of the ear tube has an inner diameter of at least 1.3 mm. In an example ear tube, a main tube of the ear tube has an inner diameter of at least 1.4 mm. In an example ear tube, a main tube of the ear tube has an inner diameter of at least 1.5 mm. In an example ear tube, a main tube of the ear tube has an inner diameter of at least 2.0 mm. In an example ear tube, a main tube of the ear tube has an inner diameter of at least 2.5 mm. In an example ear tube, a main tube of the ear tube has an inner diameter of at least 3.0 mm. In an example ear tube, a main tube of the ear tube has an inner diameter of at least 3.8 mm.

In an example ear tube, a main tube of the ear tube has an outer diameter of between 0.1 and 2.9 mm. In an example ear tube, a main tube of the ear tube has an outer diameter of between 0.1 and 2.5 mm. In an example ear tube, a main tube of the ear tube has an outer diameter of between 0.1 and 2 mm. In an example ear tube, a main tube of the ear tube has an outer diameter of between 0.1 and 1.5 mm. In an example ear tube, a main tube of the ear tube has an outer diameter of between 0.1 and 1 mm. In an example ear tube, a main tube of the ear tube has an outer diameter of between 1.0 and 3 mm. In an example ear tube, a main tube of the ear tube has an outer diameter of between 1.0 and 2 mm. In an example ear tube, a main tube of the ear tube has an outer diameter of between 1.0 and 1.5 mm. In an example ear tube, a main tube of the ear tube has an outer diameter of at least 0.1 mm. In an example ear tube, a main tube of the ear tube has an outer diameter of at least 0.5 mm. In an example ear tube, a main tube of the ear tube has an outer diameter of at least 0.6 mm. In an example ear tube, a main tube of the ear tube has an outer diameter of at least 0.7 mm. In an example ear tube, a main tube of the ear tube has an outer diameter of at least 0.8 mm. In an example ear tube, a main tube of the ear tube has an outer diameter of at least 0.9 mm. In an example ear tube, a main tube of the ear tube has an outer diameter of at least 1.0 mm. In an example ear tube, a main tube of the ear tube has an outer diameter of at least 1.1 mm. In an example ear tube, a main tube of the ear tube has an outer diameter of at least 1.2 mm. In an example ear tube, a main tube of the ear tube has an outer diameter of at least 1.3 mm. In an example ear tube, a main tube of the ear tube has an outer diameter of at least 1.4 mm. In an example ear tube, a main tube of the ear tube has an outer diameter of at least 1.5 mm. In an example ear tube, a main tube of the ear tube has an outer diameter of at least 2.0 mm. In an example ear tube, a main tube of the ear tube has an outer diameter of at least 2.5 mm. In an example ear tube, a main tube of the ear tube has an outer diameter of at least 3.0 mm. In an example ear tube, a main tube of the ear tube has an outer diameter of at least 3.8 mm.

In an example ear tube, a diameter of an inner flange is between 1.0 mm and 10.0 mm. In an example ear tube, a diameter of an inner flange is between 2.0 mm and 9.0 mm. In an example ear tube, a diameter of an inner flange is between 2.0 mm and 7.0 mm. In an example ear tube, a diameter of an inner flange is between 3.0 mm and 6.0 mm. In an example ear tube, a diameter of an inner flange is at least 1 mm. In an example ear tube, a diameter of an inner flange is at least 1.5 mm. In an example ear tube, a diameter of an inner flange is at least 2.0 mm. In an example ear tube, a diameter of an inner flange is at least 3.0 mm. In an example ear tube, a diameter of an inner flange is at least 4.0 mm. In an example ear tube, a diameter of an inner flange is at least 5.0 mm. In an example ear tube, a diameter of an inner flange is at least 6.0 mm. In an example ear tube, a diameter of an inner flange is at least 7.0 mm. In an example ear tube, a diameter of an inner flange is at least 8.0 mm. In an example ear tube, a diameter of an inner flange is at least 9.0 mm. In an example ear tube, a diameter of an inner flange is at least 10.0 mm.

In an example ear tube, a diameter of an outer flange is between 1.0 mm and 10.0 mm. In an example ear tube, a diameter of an outer flange between 2.0 mm and 9.0 mm. In an example ear tube, a diameter of an outer flange between 2.0 mm and 7.0 mm. In an example ear tube, a diameter of an outer flange or between 3.0 mm and 6.0 mm. In an example ear tube, a diameter of an outer flange at least 1 mm. In an example ear tube, a diameter of an outer flange at least 1.5 mm. In an example ear tube, a diameter of an outer flange at least 2.0 mm. In an example ear tube, a diameter of an outer flange at least 3.0 mm. In an example ear tube, a diameter of an outer flange at least 4.0 mm. In an example ear tube, a diameter of an outer flange at least 5.0 mm. In an example ear tube, a diameter of an outer flange at least 6.0 mm. In an example ear tube, a diameter of an outer flange at least 7.0 mm. In an example ear tube, a diameter of an outer flange at least 8.0 mm. In an example ear tube, a diameter of an outer flange at least 9.0 mm. In an example ear tube, a diameter of an outer flange is at least 10.0 mm.

In an example ear tube, a wall of a main tube has a thickness of between 0.1 mm and 0.5 mm. In an example ear tube, a wall of a main tube has a thickness of between 0.1 mm and 0.4 mm. In an example ear tube, a wall of a main tube has a thickness of between 0.3 mm and 0.5 mm. In an example ear tube, a wall of a main tube has a thickness of at least 0.1 mm. In an example ear tube, a wall of a main tube has a thickness of at least 0.2 mm. In an example ear tube, a wall of a main tube has a thickness of at least 0.3 mm. In an example ear tube, a wall of a main tube has a thickness of at least 0.4 mm. In an example ear tube, a wall of a main tube has a thickness of at least 0.5 mm.

In an example ear tube, a distance between a top surface of an inner flange and a bottom surface of an outer flange is between 0.05 mm and 12.0 mm. In an example ear tube, a distance between a top surface of an inner flange and a bottom surface of an outer flange is between 0.1 mm and 7.5 mm. In an example ear tube, a distance between a top surface of an inner flange and a bottom surface of an outer flange is between 1 mm and 7.5 mm. In an example ear tube, a distance between a top surface of an inner flange and a bottom surface of an outer flange is between 2.0 mm and 5.0 mm. In an example ear tube, a distance between a top surface of an inner flange and a bottom surface of an outer flange is at least 0.05 mm. In an example ear tube, a distance between a top surface of an inner flange and a bottom surface of an outer flange is at least 0.1 mm. In an example ear tube, a distance between a top surface of an inner flange and a bottom surface of an outer flange is at least 0.5 mm. In an example ear tube, a distance between a top surface of an inner flange and a bottom surface of an outer flange is at least 1.0 mm. In an example ear tube, a distance between a top surface of an inner flange and a bottom surface of an outer flange is at least 2.0 mm. In an example ear tube, a distance between a top surface of an inner flange and a bottom surface of an outer flange is at least 3.0 mm. In an example ear tube, a distance between a top surface of an inner flange and a bottom surface of an outer flange is at least 4.0 mm. In an example ear tube, a distance between a top surface of an inner flange and a bottom surface of an outer flange is at least 5.0 mm. In an example ear tube, a distance between a top surface of an inner flange and a bottom surface of an outer flange is at least 7.5 mm. In an example ear tube, a distance between a top surface of an inner flange and a bottom surface of an outer flange is at least 10.0 mm. In an example ear tube, a distance between a top surface of an inner flange and a bottom surface of an outer flange is at least 12.0 mm.

In an example ear tube, a total length of the ear tube is between 0.5 and 3 mm. In an example ear tube, a total length of the ear tube is between 0.5 and 2.5 mm. In an example ear tube, a total length of the ear tube is between 1 and 2.5 mm. In an example ear tube, a total length of the ear tube is between 1 and 2 mm. In an example ear tube, a total length of the ear tube is between 1 and 1.5 mm. In an example ear tube, a total length of the ear tube is between 2.0 mm and 5.0 mm. In an example ear tube, a total length of the ear tube is between 3.0 mm and 8.0 mm. In an example ear tube, a total length of the ear tube is at least 1.5 mm. In an example ear tube, a total length of the ear tube is at least 2.0 mm. In an example ear tube, a total length of the ear tube is at least 3.0 mm. In an example ear tube, a total length of the ear tube is at least 4.0 mm. In an example ear tube, a total length of the ear tube is at least 5.0 mm. In an example ear tube, a total length of the ear tube is at least 7.5 mm. In an example ear tube, a total length of the ear tube is at least 8.0 mm.

In an example ear tube, an outer flange and an inner flange of the ear tube are formed from a same material. In an example ear tube, an outer flange and an inner flange of the ear tube are formed from different materials. In an example ear tube, an outer flange and an inner flange of the ear tube are formed from at least two biopolymers, each of the at least two biopolymers defining a thickness of the flange. In an example ear tube, a main tube is formed from a single biopolymer. In an example ear tube, a main tube is formed from a plurality of biopolymers, wherein a first biopolymer of the plurality of biopolymers forms an inner surface of the main tube and a second biopolymer of the plurality of biopolymers is disposed circumferentially around the first biopolymer and forms an outer surface of the main tube. In an example ear tube, the ear tube includes a first biopolymer forming at least a portion of a main tube of the ear tube and at least a portion of at least one flange, and a second biopolymer forming at least a portion of the main tube and at least a portion of at least flange. In an example ear tube, an inner flange of the ear tube includes chitosan and a main tube of the ear tube and an outer flange comprise silk. In an example ear tube, the at least one material is silk fibroin. In an example ear tube, the at least one material is at least one of silk fibroin or chitosan polysaccharide. In an example ear tube, the ear tube includes silk fibroin and chitosan polysaccharide. In an example ear tube, the ear tube includes silk fibroin and alginate. In an example ear tube, the at least one material is an aqueous silk solution, an aqueous alginate solution, an aqueous fibrinogen solution, an aqueous chitosan solution, or an aqueous collagen solution.

In an example ear tube, the ear tube is formed via centrifugal molding. In an example ear tube, the ear tube is formed via thermal molding and machining. In an example ear tube, the ear tube is formed via centrifugal molding, thermal molding, and machining.

In an example ear tube, the material is at least one biopolymer and is assembled via an aqueous-based polymerization.

In an example ear tube, the ear tube includes at least one additive. In the example, the at least one additive is at least one of a bioactive molecule, a hydrophilic plasticizer, a hydrophobic plasticizer, a pigment, or a dye. The bioactive molecule is an active pharmaceutical ingredient, an antibiotic, an anti-inflammatory agent, a steroid, a growth factor, or an enzyme. The enzyme is at least one of a proteolytic enzyme, a serine protease, protease XIV, proteinase K. alpha-chymotrypsin, collagenase, MMP-1, MMP-2, or glucose oxidase. In the example, the at least one additive is dip-coated onto the ear tube. The dip-coated ear tube is subjected to water annealing. In the example, the additive is dissolved in a biopolymer solution before dip-coating. In the example, the ear tube is impregnated with the at least one additive.

In an example ear tube, the ear tube is designed to degrade on demand. Degradation commences when the ear tube is exposed to a solvent. The solvent is at least one of a glucose solution, hydrogen peroxide, carbamide peroxide, an alcohol, a calcium chelator, or EDTA.

In an example ear tube, the ear tube is a tympanostomy tube.

Disclosed herein is an ear tube including a hollow cylindrical body having at least one flanged end, wherein the ear tube includes at least one biopolymer. The ear tube has at least one characteristic centrifugal property selected from the group consisting of: i) a uniform density across at least a portion of the ear tube, ii) at least a portion of the ear tube is optically nontransparent, iii) at least a portion of the ear tube is free of exterior layering, iv) at least a portion of the ear tube is free of interior layering, v) at least a portion of the ear tube is interior-void-free, vi) at least a portion of the ear tube is free of cylindrical extrusion gaps, and vii) at least a portion of the ear tube is free of layering.

In an example ear tube, at least one of an outer flange or an inner flange is angled. The at least one of the outer flange or inner flange defines a plane which is inclined at an angle with respect to a plane disposed perpendicular to an axis of the hollow cylindrical body. The angle is between 15 degrees and 45 degrees, including but not limited to, at least 15 degrees, at least 30 degrees, or at least 45 degrees. In the example, at least one of the outer flange or inner flange includes an insertion protuberance.

In an example ear tube, the at least one flanged end includes an outer flange and an inner flange of the ear tube that are at least one of same or different with respect to at least one of a thickness, a relative angle, a shape, an orientation, or a geometry.

In an example ear tube, the hollow cylindrical body has a uniform diameter. In an example ear tube, the hollow cylindrical body has a non-uniform diameter along its length. In an example ear tube, the hollow cylindrical body tapers along its length. In an example ear tube, the ear tube has a uniform density throughout.

In an example ear tube, the ear tube comprises a cross-sectional profile that is uniform throughout the ear tube. In the example, the uniform cross-sectional profile is determined by at least one of a visual inspection, scanning electron microscopy, fluorescence microscopy, DSC, TEM, XRD, EPMA, or FTIR. In the example, the cross-sectional profile relates to at least one of a density, an absence of voids, a consistent pattern, or a chemical profile.

In an example ear tube, the hollow cylindrical body has an axial length at least several times its diameter.

In an example ear tube, the hollow cylindrical body has an inner diameter of between 0.1 and 2.9 mm. In an example ear tube, the hollow cylindrical body has an inner diameter of between 0.1 and 2.5 mm. In an example ear tube, the hollow cylindrical body has an inner diameter of between 0.1 and 2 mm. In an example ear tube, the hollow cylindrical body has an inner diameter of between 0.1 and 1.5 mm. In an example ear tube, the hollow cylindrical body has an inner diameter of between 0.1 and 1 mm. In an example ear tube, the hollow cylindrical body has an inner diameter of between 1.0 and 3 mm. In an example ear tube, the hollow cylindrical body has an inner diameter of between 1.0 and 2 mm. In an example ear tube, the hollow cylindrical body has an inner diameter of between 1.0 and 1.5 mm. In an example ear tube, the hollow cylindrical body has an inner diameter of at least 0.1 mm. In an example ear tube, the hollow cylindrical body has an inner diameter of at least 0.5 mm. In an example ear tube, the hollow cylindrical body has an inner diameter of at least 0.6 mm. In an example ear tube, the hollow cylindrical body has an inner diameter of at least 0.7 mm. In an example ear tube, the hollow cylindrical body has an inner diameter of at least 0.8 mm. In an example ear tube, the hollow cylindrical body has an inner diameter of at least 0.9 mm. In an example ear tube, the hollow cylindrical body has an inner diameter of at least 1.0 mm. In an example ear tube, the hollow cylindrical body has an inner diameter of at least 1.1 mm. In an example ear tube, the hollow cylindrical body has an inner diameter of at least 1.2 mm. In an example ear tube, the hollow cylindrical body has an inner diameter of at least 1.3 mm. In an example ear tube, the hollow cylindrical body has an inner diameter of at least 1.4 mm. In an example ear tube, the hollow cylindrical body has an inner diameter of at least 1.5 mm. In an example ear tube, the hollow cylindrical body has an inner diameter of at least 2.0 mm. In an example ear tube, the hollow cylindrical body has an inner diameter of at least 2.5 mm. In an example ear tube, the hollow cylindrical body has an inner diameter of at least 3.0 mm. In an example ear tube, the hollow cylindrical body has an inner diameter of at least 3.8 mm.

In an example ear tube, the hollow cylindrical body has an outer diameter of between 0.1 and 2.9 mm. In an example ear tube, the hollow cylindrical body has an outer diameter of between 0.1 and 2.5 mm. In an example ear tube, the hollow cylindrical body has an outer diameter of between 0.1 and 2 mm. In an example ear tube, the hollow cylindrical body has an outer diameter of between 0.1 and 1.5 mm. In an example ear tube, the hollow cylindrical body has an outer diameter of between 0.1 and 1 mm. In an example ear tube, the hollow cylindrical body has an outer diameter of between 1.0 and 3 mm. In an example ear tube, the hollow cylindrical body has an outer diameter of between 1.0 and 2 mm. In an example ear tube, the hollow cylindrical body has an outer diameter of between 1.0 and 1.5 mm. In an example ear tube, the hollow cylindrical body has an outer diameter of at least 0.1 mm. In an example ear tube, the hollow cylindrical body has an outer diameter of at least 0.5 mm. In an example ear tube, the hollow cylindrical body has an outer diameter of at least 0.6 mm. In an example ear tube, the hollow cylindrical body has an outer diameter of at least 0.7 mm. In an example ear tube, the hollow cylindrical body has an outer diameter of at least 0.8 mm. In an example ear tube, the hollow cylindrical body has an outer diameter of at least 0.9 mm. In an example ear tube, the hollow cylindrical body has an outer diameter of at least 1.0 mm. In an example ear tube, the hollow cylindrical body has an outer diameter of at least 1.1 mm. In an example ear tube, the hollow cylindrical body has an outer diameter of at least 1.2 mm. In an example ear tube, the hollow cylindrical body has an outer diameter of at least 1.3 mm. In an example ear tube, the hollow cylindrical body has an outer diameter of at least 1.4 mm. In an example ear tube, the hollow cylindrical body has an outer diameter of at least 1.5 mm. In an example ear tube, the hollow cylindrical body has an outer diameter of at least 2.0 mm. In an example ear tube, the hollow cylindrical body has an outer diameter of at least 2.5 mm. In an example ear tube, the hollow cylindrical body has an outer diameter of at least 3.0 mm. In an example ear tube, the hollow cylindrical body has an outer diameter of at least 3.8 mm.

In an example ear tube, the inner flange diameter is between 1.0 mm and 10.0 mm. In an example ear tube, the inner flange diameter is between 2.0 mm and 9.0 mm. In an example ear tube, the inner flange diameter is between 2.0 mm and 7.0 mm. In an example ear tube, the inner flange diameter is between 3.0 mm and 6.0 mm. In an example ear tube, the inner flange diameter is at least 1 mm. In an example ear tube, the inner flange diameter is at least 1.5 mm. In an example ear tube, the inner flange diameter is at least 2.0 mm. In an example ear tube, the inner flange diameter is at least 3.0 mm. In an example ear tube, the inner flange diameter is at least 4.0 mm. In an example ear tube, the inner flange diameter is at least 5.0 mm. In an example ear tube, the inner flange diameter is at least 6.0 mm. In an example ear tube, the inner flange diameter is at least 7.0 mm. In an example ear tube, the inner flange diameter is at least 8.0 mm. In an example ear tube, the inner flange diameter is at least 9.0 mm. In an example ear tube, the inner flange diameter is at least 10.0 mm.

In an example ear tube, the outer flange diameter is between 1.0 mm and 10.0 mm. In an example ear tube, the outer flange diameter is between 2.0 mm and 9.0 mm. In an example ear tube, the outer flange diameter is between 2.0 mm and 7.0 mm. In an example ear tube, the outer flange diameter is or between 3.0 mm and 6.0 mm. In an example ear tube, the outer flange diameter is at least 1 mm. In an example ear tube, the outer flange diameter is at least 1.5 mm. In an example ear tube, the outer flange diameter is at least 2.0 mm. In an example ear tube, the outer flange diameter is at least 3.0 mm. In an example ear tube, the outer flange diameter is at least 4.0 mm. In an example ear tube, the outer flange diameter is at least 5.0 mm. In an example ear tube, the outer flange diameter is at least 6.0 mm. In an example ear tube, the outer flange diameter is at least 7.0 mm. In an example ear tube, the outer flange diameter is at least 8.0 mm. In an example ear tube, the outer flange diameter is at least 9.0 mm. In an example ear tube, the outer flange diameter is at least 10.0 mm.

In an example ear tube, a wall of the hollow cylindrical body has a thickness of between 0.1 mm and 0.5 mm. In an example ear tube, a wall of the hollow cylindrical body has a thickness of between 0.1 mm and 0.4 mm. In an example ear tube, a wall of the hollow cylindrical body has a thickness of between 0.3 mm and 0.5 mm. In an example ear tube, a wall of the hollow cylindrical body has a thickness of at least 0.1 mm. In an example ear tube, a wall of the hollow cylindrical body has a thickness of at least 0.2 mm. In an example ear tube, a wall of the hollow cylindrical body has a thickness of at least 0.3 mm. In an example ear tube, a wall of the hollow cylindrical body has a thickness of at least 0.4 mm. In an example ear tube, a wall of the hollow cylindrical body has a thickness of at least 0.5 mm.

In an example ear tube, a distance between a top surface of the inner flange and a bottom surface of the outer flange is between 0.05 mm and 12.0 mm. In an example ear tube, a distance between a top surface of the inner flange and a bottom surface of the outer flange is between 0.1 mm and 7.5 mm. In an example ear tube, a distance between a top surface of the inner flange and a bottom surface of the outer flange is between 1 mm and 7.5 mm. In an example ear tube, a distance between a top surface of the inner flange and a bottom surface of the outer flange is between 2.0 mm and 5.0 mm. In an example ear tube, a distance between a top surface of the inner flange and a bottom surface of the outer flange is at least 0.05 mm. In an example ear tube, a distance between a top surface of the inner flange and a bottom surface of the outer flange is at least 0.1 mm. In an example ear tube, a distance between a top surface of the inner flange and a bottom surface of the outer flange is at least 0.5 mm. In an example ear tube, a distance between a top surface of the inner flange and a bottom surface of the outer flange is at least 1.0 mm. In an example ear tube, a distance between a top surface of the inner flange and a bottom surface of the outer flange is at least 2.0 mm. In an example ear tube, a distance between a top surface of the inner flange and a bottom surface of the outer flange is at least 3.0 mm. In an example ear tube, a distance between a top surface of the inner flange and a bottom surface of the outer flange is at least 4.0 mm. In an example ear tube, a distance between a top surface of the inner flange and a bottom surface of the outer flange is at least 5.0 mm. In an example ear tube, a distance between a top surface of the inner flange and a bottom surface of the outer flange is at least 7.5 mm. In an example ear tube, a distance between a top surface of the inner flange and a bottom surface of the outer flange is at least 10.0 mm. In an example ear tube, a distance between a top surface of the inner flange and a bottom surface of the outer flange is at least 12.0 mm.

In an example ear tube, a total length of the ear tube is between 0.5 and 3 mm. In an example ear tube, a total length of the ear tube is between 0.5 and 2.5 mm. In an example ear tube, a total length of the ear tube is between 1 and 2.5 mm. In an example ear tube, a total length of the ear tube is between 1 and 2 mm. In an example ear tube, a total length of the ear tube is between 1 and 1.5 mm. In an example ear tube, a total length of the ear tube is between 2.0 mm and 5.0 mm. In an example ear tube, a total length of the ear tube is or between 3.0 mm and 8.0 mm. In an example ear tube, a total length of the ear tube is at least 1.5 mm. In an example ear tube, a total length of the ear tube is at least 2.0 mm. In an example ear tube, a total length of the ear tube is at least 3.0 mm. In an example ear tube, a total length of the ear tube is at least 4.0 mm. In an example ear tube, a total length of the ear tube is at least 5.0 mm. In an example ear tube, a total length of the ear tube is at least 7.5 mm. In an example ear tube, a total length of the ear tube is at least 8.0 mm.

In an example ear tube, the outer flange and the inner flange are formed from the same biopolymer. In an example ear tube, the outer flange and the inner flange are formed from different biopolymers. In an example ear tube, an outer flange and an inner flange are formed from at least two biopolymers, each of the at least two biopolymers defining a thickness of the flange. In an example ear tube, the hollow cylindrical body is formed from a single biopolymer. In an example ear tube, the hollow cylindrical body is formed from a plurality of biopolymers, wherein a first biopolymer of the plurality of biopolymers forms an inner surface of the hollow cylindrical body and a second biopolymer of the plurality of biopolymers is disposed circumferentially around the first biopolymer and forms an outer surface of the hollow cylindrical body. In an example ear tube, the ear tube includes a first biopolymer forming at least a portion of the main tube and at least a portion of at least one of the flanges, and a second biopolymer forming at least a portion of the hollow cylindrical body and at least a portion of at least one of the flanges. In an example ear tube, an inner flange comprises chitosan and the hollow cylindrical body and an outer flange comprise silk. In an example ear tube, the at least one biopolymer is silk fibroin. In an example ear tube, the at least one biopolymer is at least one of silk fibroin or chitosan polysaccharide. In an example ear tube, the ear tube comprises silk fibroin and chitosan polysaccharide. In an example ear tube, the ear tube comprises silk fibroin and alginate. In an example ear tube, the at least one biopolymer is an aqueous silk solution, an aqueous alginate solution, an aqueous fibrinogen solution, an aqueous chitosan solution, or an aqueous collagen solution. In an example ear tube, the ear tube is formed via centrifugal molding. In an example ear tube, the ear tube is formed via thermal molding and machining. In an example ear tube, the ear tube is formed via centrifugal molding, thermal molding, and machining. In an example ear tube, the at least one biopolymer is assembled via an aqueous-based polymerization.

In an example ear tube, the ear tube includes at least one additive. In the example, the at least one additive is at least one of a bioactive molecule, a hydrophilic plasticizer, a hydrophobic plasticizer, a pigment, or a dye. The bioactive molecule is an active pharmaceutical ingredient, an antibiotic, an anti-inflammatory agent, a steroid, a growth factor, or an enzyme. The enzyme is at least one of a proteolytic enzyme, a serine protease, protease XIV, proteinase K. alpha-chymotrypsin, collagenase, MMP-1, MMP-2, or glucose oxidase. In the example, the at least one additive is dip-coated onto the ear tube. The dip-coated ear tube is subjected to water annealing. In the example, the additive is dissolved in a biopolymer solution before dip-coating. The ear tube is impregnated with the at least one additive.

In an example ear tube, the ear tube is designed to degrade on demand. Degradation commences when the ear tube is exposed to a solvent. The solvent is at least one of a glucose solution, hydrogen peroxide, carbamide peroxide, an alcohol, a calcium chelator, or EDTA.

In an example ear tube, the ear tube is a tympanostomy tube.

Disclosed herein is an ear tube including a main tube, the main tube extending along a center axis defining a conduit for fluid communication, the main tube having a first end and a second end, and an outer flange extending radially away from the main tube on the first end. The ear tube includes at least one biopolymer. The ear tube has at least one characteristic centrifugal property selected from the group consisting of: i) a uniform density across at least a portion of the ear tube, ii) at least a portion of the ear tube is optically nontransparent, iii) at least a portion of the ear tube is free of exterior layering, iv) at least a portion of the ear tube is free of interior layering, v) at least a portion of the ear tube is interior-void-free, vi) at least a portion of the ear tube is free of cylindrical extrusion gaps, and vii) at least a portion of the ear tube is free of layering.

In an example ear tube, at least one of the outer flange or inner flange is angled. In the example, the at least one of the outer flange or inner flange defines a plane which is inclined at an angle with respect to a plane disposed perpendicular to the axis of the main tube. The angle is between 15 degrees and 45 degrees, including but not limited to, at least 15 degrees, at least 30 degrees, or at least 45 degrees. In the example, at least one of the outer flange or inner flange includes an insertion protuberance.

In an example ear tube, the outer flange and an optional inner flange of the ear tube are at least one of same or different with respect to at least one of a thickness, a relative angle, a shape, an orientation, or a geometry.

In an example ear tube, the ear tube has a uniform diameter throughout a main tube. In an example ear tube, the ear tube has a non-uniform diameter along a length of the main tube. In an example ear tube, the ear tube tapers along a length of the main tube. In an example ear tube, the ear tube has a uniform density throughout.

In an example ear tube, the ear tube comprises a cross-sectional profile that is uniform throughout the ear tube. In the example, the uniform cross-sectional profile is determined by at least one of a visual inspection, scanning electron microscopy, fluorescence microscopy, DSC, TEM, XRD, EPMA, or FTIR. In the example, the cross-sectional profile relates to at least one of a density, an absence of voids, a consistent pattern, or a chemical profile.

In an example ear tube, the main tube has an axial length at least several times its diameter.

In an example ear tube, the main tube has an inner diameter of between 0.1 and 2.9 mm. In an example ear tube, the main tube has an inner diameter of between 0.1 and 2.5 mm. In an example ear tube, the main tube has an inner diameter of between 0.1 and 2 mm. In an example ear tube, the main tube has an inner diameter of between 0.1 and 1.5 mm. In an example ear tube, the main tube has an inner diameter of between 0.1 and 1 mm. In an example ear tube, the main tube has an inner diameter of between 1.0 and 3 mm. In an example ear tube, the main tube has an inner diameter of between 1.0 and 2 mm. In an example ear tube, the main tube has an inner diameter of between 1.0 and 1.5 mm. In an example ear tube, the main tube has an inner diameter of at least 0.1 mm. In an example ear tube, the main tube has an inner diameter of at least 0.5 mm. In an example ear tube, the main tube has an inner diameter of at least 0.6 mm. In an example ear tube, the main tube has an inner diameter of at least 0.7 mm. In an example ear tube, the main tube has an inner diameter of at least 0.8 mm. In an example ear tube, the main tube has an inner diameter of at least 0.9 mm. In an example ear tube, the main tube has an inner diameter of at least 1.0 mm. In an example ear tube, the main tube has an inner diameter of at least 1.1 mm. In an example ear tube, the main tube has an inner diameter of at least 1.2 mm. In an example ear tube, the main tube has an inner diameter of at least 1.3 mm. In an example ear tube, the main tube has an inner diameter of at least 1.4 mm. In an example ear tube, the main tube has an inner diameter of at least 1.5 mm. In an example ear tube, the main tube has an inner diameter of at least 2.0 mm. In an example ear tube, the main tube has an inner diameter of at least 2.5 mm. In an example ear tube, the main tube has an inner diameter of at least 3.0 mm. In an example ear tube, the main tube has an inner diameter of at least 3.8 mm.

In an example ear tube, the main tube has an outer diameter of between 0.1 and 2.9 mm. In an example ear tube, the main tube has an outer diameter of between 0.1 and 2.5 mm. In an example ear tube, the main tube has an outer diameter of between 0.1 and 2 mm. In an example ear tube, the main tube has an outer diameter of between 0.1 and 1.5 mm. In an example ear tube, the main tube has an outer diameter of between 0.1 and 1 mm. In an example ear tube, the main tube has an outer diameter of between 1.0 and 3 mm. In an example ear tube, the main tube has an outer diameter of between 1.0 and 2 mm. In an example ear tube, the main tube has an outer diameter of or between 1.0 and 1.5 mm. In an example ear tube, the main tube has an outer diameter of at least 0.1 mm. In an example ear tube, the main tube has an outer diameter of at least 0.5 mm. In an example ear tube, the main tube has an outer diameter of at least 0.6 mm. In an example ear tube, the main tube has an outer diameter of at least 0.7 mm. In an example ear tube, the main tube has an outer diameter of at least 0.8 mm. In an example ear tube, the main tube has an outer diameter of at least 0.9 mm. In an example ear tube, the main tube has an outer diameter of at least 1.0 mm. In an example ear tube, the main tube has an outer diameter of at least 1.1 mm. In an example ear tube, the main tube has an outer diameter of at least 1.2 mm. In an example ear tube, the main tube has an outer diameter of at least 1.3 mm. In an example ear tube, the main tube has an outer diameter of at least 1.4 mm. In an example ear tube, the main tube has an outer diameter of at least 1.5 mm. In an example ear tube, the main tube has an outer diameter of at least 2.0 mm. In an example ear tube, the main tube has an outer diameter of at least 2.5 mm. In an example ear tube, the main tube has an outer diameter of at least 3.0 mm. In an example ear tube, the main tube has an outer diameter of at least 3.8 mm.

In an example ear tube, the inner flange diameter is between 1.0 mm and 10.0 mm. In an example ear tube, the inner flange diameter is between 2.0 mm and 9.0 mm. In an example ear tube, the inner flange diameter is between 2.0 mm and 7.0 mm. In an example ear tube, the inner flange diameter is between 3.0 mm and 6.0 mm. In an example ear tube, the inner flange diameter is at least 1 mm. In an example ear tube, the inner flange diameter is at least 1.5 mm. In an example ear tube, the inner flange diameter is at least 2.0 mm. In an example ear tube, the inner flange diameter is at least 3.0 mm. In an example ear tube, the inner flange diameter is at least 4.0 mm. In an example ear tube, the inner flange diameter is at least 5.0 mm. In an example ear tube, the inner flange diameter is at least 6.0 mm. In an example ear tube, the inner flange diameter is at least 7.0 mm. In an example ear tube, the inner flange diameter is at least 8.0 mm. In an example ear tube, the inner flange diameter is at least 9.0 mm. In an example ear tube, the inner flange diameter is or at least 10.0 mm.

In an example ear tube, the outer flange diameter is between 1.0 mm and 10.0 mm. In an example ear tube, the outer flange diameter is between 2.0 mm and 9.0 mm. In an example ear tube, the outer flange diameter is between 2.0 mm and 7.0 mm. In an example ear tube, the outer flange diameter is between 3.0 mm and 6.0 mm. In an example ear tube, the outer flange diameter is at least 1 mm. In an example ear tube, the outer flange diameter is at least 1.5 mm. In an example ear tube, the outer flange diameter is at least 2.0 mm. In an example ear tube, the outer flange diameter is at least 3.0 mm. In an example ear tube, the outer flange diameter is at least 4.0 mm. In an example ear tube, the outer flange diameter is at least 5.0 mm. In an example ear tube, the outer flange diameter is at least 6.0 mm. In an example ear tube, the outer flange diameter is at least 7.0 mm. In an example ear tube, the outer flange diameter is at least 8.0 mm. In an example ear tube, the outer flange diameter is at least 9.0 mm. In an example ear tube, the outer flange diameter is at least 10.0 mm.

In an example ear tube, a wall of the main tube has a thickness of between 0.1 mm and 0.5 mm. In an example ear tube, a wall of the main tube has a thickness of between 0.1 mm and 0.4 mm. In an example ear tube, a wall of the main tube has a thickness of between 0.3 mm and 0.5 mm. In an example ear tube, a wall of the main tube has a thickness of at least 0.1 mm. In an example ear tube, a wall of the main tube has a thickness of at least 0.2 mm. In an example ear tube, a wall of the main tube has a thickness of at least 0.3 mm. In an example ear tube, a wall of the main tube has a thickness of at least 0.4 mm. In an example ear tube, a wall of the main tube has a thickness of at least 0.5 mm.

In an example ear tube, a distance between a top surface of an optional inner flange and a bottom surface of the outer flange is between 0.05 mm and 12.0 mm. In an example ear tube, a distance between a top surface of an optional inner flange and a bottom surface of the outer flange is between 0.1 mm and 7.5 mm. In an example ear tube, a distance between a top surface of an optional inner flange and a bottom surface of the outer flange is between 1 mm and 7.5 mm. In an example ear tube, a distance between a top surface of an optional inner flange and a bottom surface of the outer flange is between 2.0 mm and 5.0 mm. In an example ear tube, a distance between a top surface of an optional inner flange and a bottom surface of the outer flange is at least 0.05 mm. In an example ear tube, a distance between a top surface of an optional inner flange and a bottom surface of the outer flange is at least 0.1 mm. In an example ear tube, a distance between a top surface of an optional inner flange and a bottom surface of the outer flange is at least 0.5 mm. In an example ear tube, a distance between a top surface of an optional inner flange and a bottom surface of the outer flange is at least 1.0 mm. In an example ear tube, a distance between a top surface of an optional inner flange and a bottom surface of the outer flange is at least 2.0 mm. In an example ear tube, a distance between a top surface of an optional inner flange and a bottom surface of the outer flange is at least 3.0 mm. In an example ear tube, a distance between a top surface of an optional inner flange and a bottom surface of the outer flange is at least 4.0 mm. In an example ear tube, a distance between a top surface of an optional inner flange and a bottom surface of the outer flange is at least 5.0 mm. In an example ear tube, a distance between a top surface of an optional inner flange and a bottom surface of the outer flange is at least 7.5 mm. In an example ear tube, a distance between a top surface of an optional inner flange and a bottom surface of the outer flange is at least 10.0 mm. In an example ear tube, a distance between a top surface of an optional inner flange and a bottom surface of the outer flange is at least 12.0 mm.

In an example ear tube, a total length of the ear tube is between 0.5 and 3 mm. In an example ear tube, a total length of the ear tube is between 0.5 and 2.5 mm. In an example ear tube, a total length of the ear tube is between 1 and 2.5 mm. In an example ear tube, a total length of the ear tube is between 1 and 2 mm. In an example ear tube, a total length of the ear tube is between 1 and 1.5 mm. In an example ear tube, a total length of the ear tube is between 2.0 mm and 5.0 mm. In an example ear tube, a total length of the ear tube is or between 3.0 mm and 8.0 mm. In an example ear tube, a total length of the ear tube is at least 1.5 mm. In an example ear tube, a total length of the ear tube is at least 2.0 mm. In an example ear tube, a total length of the ear tube is at least 3.0 mm. In an example ear tube, a total length of the ear tube is at least 4.0 mm. In an example ear tube, a total length of the ear tube is at least 5.0 mm. In an example ear tube, a total length of the ear tube is at least 7.5 mm. In an example ear tube, a total length of the ear tube is at least 8.0 mm.

In an example ear tube, the outer flange and an optional inner flange are formed from a same biopolymer.

In an example ear tube, the outer flange and an optional inner flange are formed from different biopolymers.

In an example ear tube, the outer flange and the inner flange are formed from at least two biopolymers, each of the at least two biopolymers defining a thickness of the flange.

In an example ear tube, the main tube is formed from a single biopolymer. In an example ear tube, the main tube is formed from a plurality of biopolymers, wherein a first biopolymer of the plurality of biopolymers forms an inner surface of the main tube and a second biopolymer of the plurality of biopolymers is disposed circumferentially around the first biopolymer and forms an outer surface of the main tube. In an example ear tube, the ear tube includes a first biopolymer forming at least a portion of the main tube and at least a portion of at least one of the flanges, and a second biopolymer forming at least a portion of the main tube and at least a portion of at least one of the flanges. In an example ear tube, an optional inner flange includes chitosan and the main tube and outer flange include silk. In an example ear tube, the at least one biopolymer is silk fibroin. In an example ear tube, the at least one biopolymer is at least one of silk fibroin or chitosan polysaccharide. In an example ear tube, the ear tube includes silk fibroin and chitosan polysaccharide. In an example ear tube, the ear tube includes silk fibroin and alginate. In an example ear tube, the at least one biopolymer is an aqueous silk solution, an aqueous alginate solution, an aqueous fibrinogen solution, an aqueous chitosan solution, or an aqueous collagen solution.

In an example ear tube, the ear tube is formed via centrifugal molding. In an example ear tube, the ear tube is formed via thermal molding and machining. In an example ear tube, the ear tube is formed via centrifugal molding, thermal molding, and machining. In an example ear tube, the at least one biopolymer is assembled via an aqueous-based polymerization.

In an example ear tube, the ear tube includes at least one additive. In the example, the at least one additive is at least one of a bioactive molecule, a hydrophilic plasticizer, a hydrophobic plasticizer, a pigment, or a dye. The bioactive molecule is an active pharmaceutical ingredient, an antibiotic, an anti-inflammatory agent, a steroid, a growth factor, or an enzyme. The enzyme is at least one of a proteolytic enzyme, a serine protease, protease XIV, proteinase K. alpha-chymotrypsin, collagenase, MMP-1, MMP-2, or glucose oxidase. The at least one additive is dip-coated onto the ear tube. The dip-coated ear tube is subjected to water annealing. In the example, the additive is dissolved in a biopolymer solution before dip-coating. In the example, the ear tube is impregnated with the at least one additive.

In an example ear tube, the ear tube is designed to degrade on demand. In the example, degradation commences when the ear tube is exposed to a solvent. The solvent is at least one of a glucose solution, hydrogen peroxide, carbamide peroxide, an alcohol, a calcium chelator, or EDTA.

In an example ear tube, the ear tube is a tympanostomy tube.

Disclosed herein is an ear tube including a main tube, the main tube extending along a center axis defining a conduit for fluid communication, the main tube having a first end and a second end, an outer flange extending radially away from the main tube on a first end, an inner flange extending radially away from the main tube on a second end, and a frustoconical portion of the main tube extending longitudinally from at least one of the first end or the second end, the frustoconical portion extending beyond a width of the flange on the at least one of the first end or the second end, wherein the ear tube includes at least one biopolymer. The ear tube has at least one characteristic centrifugal property selected from the group consisting of: i) a uniform density across at least a portion of the ear tube, ii) at least a portion of the ear tube is optically nontransparent, iii) at least a portion of the ear tube is free of exterior layering, iv) at least a portion of the ear tube is free of interior layering, v) at least a portion of the ear tube is interior-void-free, vi) at least a portion of the ear tube is free of cylindrical extrusion gaps, and vii) at least a portion of the ear tube is free of layering.

In an example ear tube, at least one of the outer flange or inner flange is angled. In the example, the at least one of the outer flange or inner flange defines a plane which is inclined at an angle with respect to a plane disposed perpendicular to the axis of the main tube. The angle is between 15 degrees and 45 degrees, including but not limited to, at least 15 degrees, at least 30 degrees, or at least 45 degrees.

In an example ear tube, at least one of the outer flange or inner flange comprises an insertion protuberance.

In an example ear tube, an outer flange and an inner flange of the ear tube are at least one of same or different with respect to at least one of a thickness, a relative angle, a shape, an orientation, or a geometry.

In an example ear tube, the ear tube has a uniform diameter throughout a main tube. In an example ear tube, the ear tube has a non-uniform diameter along a length of the main tube. In an example ear tube, the ear tube tapers along a length of the main tube. In an example ear tube, the ear tube has a uniform density throughout.

In an example ear tube, the ear tube includes a cross-sectional profile that is uniform throughout the ear tube. In the example, the uniform cross-sectional profile is determined by at least one of a visual inspection, scanning electron microscopy, fluorescence microscopy, DSC, TEM, XRD, EPMA, or FTIR. In the example, the cross-sectional profile relates to at least one of a density, an absence of voids, a consistent pattern, or a chemical profile.

In an example ear tube, the main tube has an axial length at least several times its diameter.

In an example ear tube, the main tube has an inner diameter of between 0.1 and 2.9 mm. In an example ear tube, the main tube has an inner diameter of between 0.1 and 2.5 mm. In an example ear tube, the main tube has an inner diameter of between 0.1 and 2 mm. In an example ear tube, the main tube has an inner diameter of between 0.1 and 1.5 mm. In an example ear tube, the main tube has an inner diameter of between 0.1 and 1 mm. In an example ear tube, the main tube has an inner diameter of between 1.0 and 3 mm. In an example ear tube, the main tube has an inner diameter of between 1.0 and 2 mm. In an example ear tube, the main tube has an inner diameter of between 1.0 and 1.5 mm. In an example ear tube, the main tube has an inner diameter of at least 0.1 mm. In an example ear tube, the main tube has an inner diameter of at least 0.5 mm. In an example ear tube, the main tube has an inner diameter of at least 0.6 mm. In an example ear tube, the main tube has an inner diameter of at least 0.7 mm. In an example ear tube, the main tube has an inner diameter of at least 0.8 mm. In an example ear tube, the main tube has an inner diameter of at least 0.9 mm. In an example ear tube, the main tube has an inner diameter of at least 1.0 mm. In an example ear tube, the main tube has an inner diameter of at least 1.1 mm. In an example ear tube, the main tube has an inner diameter of at least 1.2 mm. In an example ear tube, the main tube has an inner diameter of at least 1.3 mm. In an example ear tube, the main tube has an inner diameter of at least 1.4 mm. In an example ear tube, the main tube has an inner diameter of at least 1.5 mm. In an example ear tube, the main tube has an inner diameter of at least 2.0 mm. In an example ear tube, the main tube has an inner diameter of at least 2.5 mm. In an example ear tube, the main tube has an inner diameter of at least 3.0 mm. In an example ear tube, the main tube has an inner diameter of at least 3.8 mm.

In an example ear tube, the main tube has an outer diameter of between 0.1 and 2.9 mm. In an example ear tube, the main tube has an outer diameter of between 0.1 and 2.5 mm. In an example ear tube, the main tube has an outer diameter of between 0.1 and 2 mm. In an example ear tube, the main tube has an outer diameter of between 0.1 and 1.5 mm. In an example ear tube, the main tube has an outer diameter of between 0.1 and 1 mm. In an example ear tube, the main tube has an outer diameter of between 1.0 and 3 mm. In an example ear tube, the main tube has an outer diameter of between 1.0 and 2 mm. In an example ear tube, the main tube has an outer diameter of or between 1.0 and 1.5 mm. In an example ear tube, the main tube has an outer diameter of at least 0.1 mm. In an example ear tube, the main tube has an outer diameter of at least 0.5 mm. In an example ear tube, the main tube has an outer diameter of at least 0.6 mm. In an example ear tube, the main tube has an outer diameter of at least 0.7 mm. In an example ear tube, the main tube has an outer diameter of at least 0.8 mm. In an example ear tube, the main tube has an outer diameter of at least 0.9 mm. In an example ear tube, the main tube has an outer diameter of at least 1.0 mm. In an example ear tube, the main tube has an outer diameter of at least 1.1 mm. In an example ear tube, the main tube has an outer diameter of at least 1.2 mm. In an example ear tube, the main tube has an outer diameter of at least 1.3 mm. In an example ear tube, the main tube has an outer diameter of at least 1.4 mm. In an example ear tube, the main tube has an outer diameter of at least 1.5 mm. In an example ear tube, the main tube has an outer diameter of at least 2.0 mm. In an example ear tube, the main tube has an outer diameter of at least 2.5 mm. In an example ear tube, the main tube has an outer diameter of at least 3.0 mm. In an example ear tube, the main tube has an outer diameter of at least 3.8 mm.

In an example ear tube, the inner flange diameter is between 1.0 mm and 10.0 mm. In an example ear tube, the inner flange diameter is between 2.0 mm and 9.0 mm. In an example ear tube, the inner flange diameter is between 2.0 mm and 7.0 mm. In an example ear tube, the inner flange diameter is between 3.0 mm and 6.0 mm. In an example ear tube, the inner flange diameter is at least 1 mm. In an example ear tube, the inner flange diameter is at least 1.5 mm. In an example ear tube, the inner flange diameter is at least 2.0 mm. In an example ear tube, the inner flange diameter is at least 3.0 mm. In an example ear tube, the inner flange diameter is at least 4.0 mm. In an example ear tube, the inner flange diameter is at least 5.0 mm. In an example ear tube, the inner flange diameter is at least 6.0 mm. In an example ear tube, the inner flange diameter is at least 7.0 mm. In an example ear tube, the inner flange diameter is at least 8.0 mm. In an example ear tube, the inner flange diameter is at least 9.0 mm. In an example ear tube, the inner flange diameter is or at least 10.0 mm.

In an example ear tube, the outer flange diameter is between 1.0 mm and 10.0 mm. In an example ear tube, the outer flange diameter is between 2.0 mm and 9.0 mm. In an example ear tube, the outer flange diameter is between 2.0 mm and 7.0 mm. In an example ear tube, the outer flange diameter is between 3.0 mm and 6.0 mm. In an example ear tube, the outer flange diameter is at least 1 mm. In an example ear tube, the outer flange diameter is at least 1.5 mm. In an example ear tube, the outer flange diameter is at least 2.0 mm. In an example ear tube, the outer flange diameter is at least 3.0 mm. In an example ear tube, the outer flange diameter is at least 4.0 mm. In an example ear tube, the outer flange diameter is at least 5.0 mm. In an example ear tube, the outer flange diameter is at least 6.0 mm. In an example ear tube, the outer flange diameter is at least 7.0 mm. In an example ear tube, the outer flange diameter is at least 8.0 mm. In an example ear tube, the outer flange diameter is at least 9.0 mm. In an example ear tube, the outer flange diameter is at least 10.0 mm.

In an example ear tube, a wall of the main tube has a thickness of between 0.1 mm and 0.5 mm. In an example ear tube, a wall of the main tube has a thickness of between 0.1 mm and 0.4 mm. In an example ear tube, a wall of the main tube has a thickness of between 0.3 mm and 0.5 mm. In an example ear tube, a wall of the main tube has a thickness of at least 0.1 mm. In an example ear tube, a wall of the main tube has a thickness of at least 0.2 mm. In an example ear tube, a wall of the main tube has a thickness of at least 0.3 mm. In an example ear tube, a wall of the main tube has a thickness of at least 0.4 mm. In an example ear tube, a wall of the main tube has a thickness of at least 0.5 mm.

In an example ear tube, a distance between a top surface of an inner flange and a bottom surface of the outer flange is between 0.05 mm and 12.0 mm. In an example ear tube, a distance between a top surface of an inner flange and a bottom surface of the outer flange is between 0.1 mm and 7.5 mm. In an example ear tube, a distance between a top surface of an inner flange and a bottom surface of the outer flange is between 1 mm and 7.5 mm. In an example ear tube, a distance between a top surface of an inner flange and a bottom surface of the outer flange is between 2.0 mm and 5.0 mm. In an example ear tube, a distance between a top surface of an inner flange and a bottom surface of the outer flange is at least 0.05 mm. In an example ear tube, a distance between a top surface of an inner flange and a bottom surface of the outer flange is at least 0.1 mm. In an example ear tube, a distance between a top surface of an inner flange and a bottom surface of the outer flange is at least 0.5 mm. In an example ear tube, a distance between a top surface of an inner flange and a bottom surface of the outer flange is at least 1.0 mm. In an example ear tube, a distance between a top surface of an inner flange and a bottom surface of the outer flange is at least 2.0 mm. In an example ear tube, a distance between a top surface of an inner flange and a bottom surface of the outer flange is at least 3.0 mm. In an example ear tube, a distance between a top surface of an inner flange and a bottom surface of the outer flange is at least 4.0 mm. In an example ear tube, a distance between a top surface of an inner flange and a bottom surface of the outer flange is at least 5.0 mm. In an example ear tube, a distance between a top surface of an inner flange and a bottom surface of the outer flange is at least 7.5 mm. In an example ear tube, a distance between a top surface of an inner flange and a bottom surface of the outer flange is at least 10.0 mm. In an example ear tube, a distance between a top surface of an inner flange and a bottom surface of the outer flange is at least 12.0 mm.

In an example ear tube, a total length of the ear tube is between 0.5 and 3 mm. In an example ear tube, a total length of the ear tube is between 0.5 and 2.5 mm. In an example ear tube, a total length of the ear tube is between 1 and 2.5 mm. In an example ear tube, a total length of the ear tube is between 1 and 2 mm. In an example ear tube, a total length of the ear tube is between 1 and 1.5 mm. In an example ear tube, a total length of the ear tube is between 2.0 mm and 5.0 mm. In an example ear tube, a total length of the ear tube is or between 3.0 mm and 8.0 mm. In an example ear tube, a total length of the ear tube is at least 1.5 mm. In an example ear tube, a total length of the ear tube is at least 2.0 mm. In an example ear tube, a total length of the ear tube is at least 3.0 mm. In an example ear tube, a total length of the ear tube is at least 4.0 mm. In an example ear tube, a total length of the ear tube is at least 5.0 mm. In an example ear tube, a total length of the ear tube is at least 7.5 mm. In an example ear tube, a total length of the ear tube is at least 8.0 mm.

In an example ear tube, the outer flange and the inner flange are formed from a same biopolymer.

In an example ear tube, the outer flange and the inner flange are formed from different biopolymers.

In an example ear tube, the outer flange and the inner flange are formed from at least two biopolymers, each of the at least two biopolymers defining a thickness of the flange. In an example ear tube, the main tube is formed from a single biopolymer. In an example ear tube, the main tube is formed from a plurality of biopolymers, wherein a first biopolymer of the plurality of biopolymers forms an inner surface of the main tube and a second biopolymer of the plurality of biopolymers is disposed circumferentially around the first biopolymer and forms an outer surface of the main tube. In an example ear tube, the ear tube includes a first biopolymer forming at least a portion of the main tube and at least a portion of at least one of the flanges, and a second biopolymer forming at least a portion of the main tube and at least a portion of at least one of the flanges. In an example ear tube, the inner flange comprises chitosan and the main tube and outer flange comprise silk. In an example ear tube, the at least one biopolymer is silk fibroin. In an example ear tube, the at least one biopolymer is at least one of silk fibroin or chitosan polysaccharide. In an example ear tube, the ear tube includes silk fibroin and chitosan polysaccharide. In an example ear tube, the ear tube includes silk fibroin and alginate. In an example ear tube, the at least one biopolymer is an aqueous silk solution, an aqueous alginate solution, an aqueous fibrinogen solution, an aqueous chitosan solution, or an aqueous collagen solution.

In an example ear tube, the ear tube is formed via centrifugal molding. In an example ear tube, the ear tube is formed via thermal molding and machining. In an example ear tube, the ear tube is formed via centrifugal molding, thermal molding, and machining. In an example ear tube, the at least one biopolymer is assembled via an aqueous-based polymerization.

In an example ear tube, the ear tube comprises at least one additive. In the example, the at least one additive is at least one of a bioactive molecule, a hydrophilic plasticizer, a hydrophobic plasticizer, a pigment, or a dye. In the example, the bioactive molecule is an active pharmaceutical ingredient, an antibiotic, an anti-inflammatory agent, a steroid, a growth factor, or an enzyme. The enzyme is at least one of a proteolytic enzyme, a serine protease, protease XIV, proteinase K. alpha-chymotrypsin, collagenase, MMP-1, MMP-2, or glucose oxidase. In the example, the at least one additive is dip-coated onto the ear tube. The dip-coated ear tube is subjected to water annealing. In the example, the additive is dissolved in a biopolymer solution before dip-coating. In the example, the ear tube is impregnated with the at least one additive.

In an example ear tube, the ear tube is designed to degrade on demand. In the example, degradation commences when the ear tube is exposed to a solvent. The solvent is at least one of a glucose solution, hydrogen peroxide, carbamide peroxide, an alcohol, a calcium chelator, or EDTA.

In an example ear tube, the ear tube is a tympanostomy tube.

Disclosed herein is a method of forming an ear tube including depositing at least one aqueous biopolymer solution into an ear tube mold, subjecting the ear tube mold and its contents to a first centrifugation, depositing a polymerization bath into the ear tube mold and subjecting the ear tube mold and its contents to a second centrifugation to polymerize the aqueous biopolymer solution into a shape of an ear tube, and drying the ear tube.

In an example method, the ear tube dwells in the mold after the second centrifugation to complete polymerization before the drying.

In an example method, the at least one aqueous biopolymer solution is an aqueous silk solution, an aqueous alginate solution, an aqueous fibrinogen solution, an aqueous chitosan solution, or an aqueous collagen solution.

In an example method, the method further includes subjecting the ear tube to at least one of cross-linking, dipcoating, covalent bonding, heat treatment, pressure treatment, water annealing, sterilization, surface modifications, chemical etching, chemical smoothing, or physical smoothing.

Disclosed herein is a method of degrading an ear tube including exposing an ear tube to a solvent, wherein the ear tube comprises at least one biopolymer having at least one characteristic centrifugal property selected from the group consisting of: i) a uniform density across at least a portion of the ear tube, ii) at least a portion of the ear tube is optically nontransparent, iii) at least a portion of the ear tube is free of exterior layering, iv) at least a portion of the ear tube is free of interior layering, v) at least a portion of the ear tube is interior-void-free, vi) at least a portion of the ear tube is free of cylindrical extrusion gaps, and vii) at least a portion of the ear tube is free of layering.

In an example method, the solvent is at least one of a glucose solution, hydrogen peroxide, carbamide peroxide, an alcohol, a calcium chelator, or EDTA.

In an example method, exposing is over a period of at least one of minutes, hours, or days.

In any of the disclosed ear tubes or methods herein, there is a uniform density across the at least a portion of the ear tube. In any of the disclosed ear tubes or methods herein, the at least a portion of the ear tube is optically nontransparent. In any of the disclosed ear tubes or methods herein, the at least a portion of the ear tube is free of exterior layering. In any of the disclosed ear tubes or methods herein, the at least a portion of the ear tube is free of interior layering. In any of the disclosed ear tubes or methods herein, the at least a portion of the ear tube is interior-void-free. In any of the disclosed ear tubes or methods herein, the at least a portion of the ear tube is free of cylindrical extrusion gaps. In any of the disclosed ear tubes or methods herein, the at least a portion of the ear tube is free of layering.

Disclosed herein is an ear tube including chitosan. The ear tube has at least one characteristic centrifugal property selected from the group consisting of: i) a uniform density across at least a portion of the ear tube, ii) at least a portion of the ear tube is optically nontransparent, iii) at least a portion of the ear tube is free of exterior layering, iv) at least a portion of the ear tube is free of interior layering, v) at least a portion of the ear tube is interior-void-free, vi) at least a portion of the ear tube is free of cylindrical extrusion gaps, and vii) at least a portion of the ear tube is free of layering.

In an example ear tube, at least one of an outer flange or an inner flange of the ear tube is angled. In the example, the at least one of the outer flange or inner flange defines a plane which is inclined at an angle with respect to a plane disposed perpendicular to an axis of a main tube of the ear tube. The angle is between 15 degrees and 45 degrees, including but not limited to, at least 15 degrees, at least 30 degrees, or at least 45 degrees. In the example, at least one of the outer flange or inner flange includes an insertion protuberance.

In an example ear tube, an outer flange and an inner flange of the ear tube are at least one of same or different with respect to at least one of a thickness, a relative angle, a shape, an orientation, or a geometry. In an example ear tube, the ear tube has a uniform diameter throughout its length. In an example ear tube, the ear tube has a non-uniform diameter along its length. In an example ear tube, the ear tube tapers along its length. In an example ear tube, the ear tube has a uniform density throughout.

In an example ear tube, the ear tube includes a cross-sectional profile that is uniform throughout the ear tube. In the example, the uniform cross-sectional profile is determined by at least one of a visual inspection, scanning electron microscopy, fluorescence microscopy, DSC, TEM, XRD, EPMA, or FTIR. In the example, the cross-sectional profile relates to at least one of a density, an absence of voids, a consistent pattern, or a chemical profile.

In an example ear tube, a main tube of the ear tube has an axial length at least several times its diameter.

In an example ear tube, a main tube of the ear tube has an inner diameter of between 0.1 and 2.9 mm, between 0.1 and 2.5 mm, between 0.1 and 2 mm, between 0.1 and 1.5 mm, between 0.1 and 1 mm, between 1.0 and 3 mm, between 1.0 and 2 mm, or between 1.0 and 1.5 mm, including but not limited to, at least 0.1 mm, at least 0.5 mm, at least 0.6 mm, at least 0.7 mm, at least 0.8 mm, at least 0.9 mm, at least 1.0 mm, at least 1.1 mm, at least 1.2 mm, at least 1.3 mm, at least 1.4 mm, at least 1.5 mm, at least 2.0 mm, at least 2.5 mm, at least 3.0 mm, or at least 3.8 mm.

In an example ear tube, a main tube of the ear tube has an outer diameter of between 0.1 and 2.9 mm, between 0.1 and 2.5 mm, between 0.1 and 2 mm, between 0.1 and 1.5 mm, between 0.1 and 1 mm, between 1.0 and 3 mm, between 1.0 and 2 mm, or between 1.0 and 1.5 mm, including but not limited to, at least 0.1 mm, at least 0.5 mm, at least 0.6 mm, at least 0.7 mm, at least 0.8 mm, at least 0.9 mm, at least 1.0 mm, at least 1.1 mm, at least 1.2 mm, at least 1.3 mm, at least 1.4 mm, at least 1.5 mm, at least 2.0 mm, at least 2.5 mm, at least 3.0 mm, or at least 3.8 mm.

In an example ear tube, the ear tube has an inner flange, the inner flange diameter is between 1.0 mm and 10.0 mm, between 2.0 mm and 9.0 mm, between 2.0 mm and 7.0 mm, or between 3.0 mm and 6.0 mm, including but not limited to, at least 1 mm, at least 1.5 mm, at least 2.0 mm, at least 3.0 mm, at least 4.0 mm, at least 5.0 mm, at least 6.0 mm, at least 7.0 mm, at least 8.0 mm, at least 9.0 mm, or at least 10.0 mm.

In an example ear tube, the ear tube has an outer flange, the outer flange diameter is between 1.0 mm and 10.0 mm, between 2.0 mm and 9.0 mm, between 2.0 mm and 7.0 mm, or between 3.0 mm and 6.0 mm, including but not limited to, at least 1 mm, at least 1.5 mm, at least 2.0 mm, at least 3.0 mm, at least 4.0 mm, at least 5.0 mm, at least 6.0 mm, at least 7.0 mm, at least 8.0 mm, at least 9.0 mm, or at least 10.0 mm.

In an example ear tube, a wall of a main tube of the ear tube has a thickness of between 0.1 mm and 0.5 mm, between 0.1 mm and 0.4 mm, or between 0.3 mm and 0.5 mm, including but not limited to, at least 0.1 mm, at least 0.2 mm, at least 0.3 mm, at least 0.4 mm, or at least 0.5 mm.

In an example ear tube, a distance between a top surface of an inner flange of the ear tube and a bottom surface of an outer flange of the ear tube is between 0.05 mm and 12.0 mm, between 0.1 mm and 7.5 mm, between 1 mm and 7.5 mm, between 2.0 mm and 5.0 mm, including but not limited to, at least 0.05 mm, at least 0.1 mm, at least 0.5 mm, at least 1.0 mm, at least 2.0 mm. at least 3.0 mm, at least 4.0 mm, at least 5.0 mm, at least 7.5 mm, at least 10.0 mm, or at least 12.0 mm.

In an example ear tube, a total length of the ear tube is between 0.5 and 3 mm, between 0.5 and 2.5 mm, between 1 and 2.5 mm, between 1 and 2 mm, between 1 and 1.5 mm, between 2.0 mm and 5.0 mm, or between 3.0 mm and 8.0 mm, including but not limited to, at least 1.5 mm, at least 2.0 mm. at least 3.0 mm, at least 4.0 mm, at least 5.0 mm, at least 7.5 mm, or at least 8.0 mm.

In an example ear tube, an outer flange and an inner flange of the ear tube are both formed from chitosan.

In an example ear tube, an outer flange and an inner flange of the ear tube are formed from different biopolymers, at least one of the biopolymers comprising chitosan.

In an example ear tube, an outer flange and an inner flange of the ear tube are formed from at least two biopolymers, each of the at least two biopolymers defining a thickness of the flange.

In an example ear tube, a main tube of the ear tube is formed from chitosan.

In an example ear tube, a main tube of the ear tube is formed from a plurality of biopolymers, wherein a first biopolymer of the plurality of biopolymers forms an inner surface of the main tube and a second biopolymer of the plurality of biopolymers is disposed circumferentially around the first biopolymer and forms an outer surface of the main tube, and wherein at least one of the first biopolymer or the second biopolymer is chitosan.

In an example ear tube, the ear tube includes a first biopolymer forming at least a portion of a main tube of the ear tube and at least a portion of at least one flange, and a second biopolymer forming at least a portion of the main tube of the ear tube and at least a portion of at least one flange, and wherein at least one of the first biopolymer or the second biopolymer is chitosan.

In an example ear tube, an inner flange of the ear tube includes chitosan and a main tube of the ear tube and outer flange comprise silk.

In an example ear tube, the ear tube comprises at least one biopolymer, the at least one biopolymer is silk fibroin. In an example ear tube, the ear tube includes at least one biopolymer, the at least one biopolymer is at least one of silk fibroin or chitosan polysaccharide. In an example ear tube, the ear tube includes silk fibroin and chitosan polysaccharide. In an example ear tube, the ear tube includes silk fibroin and alginate. In an example ear tube, the ear tube includes at least one biopolymer, the at least one biopolymer is an aqueous silk solution, an aqueous alginate solution, an aqueous fibrinogen solution, an aqueous chitosan solution, or an aqueous collagen solution.

In an example ear tube, the ear tube is formed via centrifugal molding. In an example ear tube, the ear tube is formed via thermal molding and machining. In an example ear tube, the ear tube is formed via centrifugal molding, thermal molding, and machining.

In an example ear tube, the ear tube includes at least one biopolymer, the at least one biopolymer is assembled via an aqueous-based polymerization.

In an example ear tube, the ear tube includes at least one additive. In the example, the at least one additive is at least one of a bioactive molecule, a hydrophilic plasticizer, a hydrophobic plasticizer, a pigment, or a dye. The bioactive molecule is an active pharmaceutical ingredient, an antibiotic, an anti-inflammatory agent, a steroid, a growth factor, or an enzyme. The enzyme is at least one of a proteolytic enzyme, a serine protease, protease XIV, proteinase K. alpha-chymotrypsin, collagenase, MMP-1, MMP-2, or glucose oxidase. In the example, the at least one additive is dip-coated onto the ear tube. The dip-coated ear tube is subjected to water annealing. In the example, the additive is dissolved in a biopolymer solution before dip-coating. In the example, the ear tube is impregnated with the at least one additive.

In an example ear tube, the ear tube is designed to degrade on demand. In the example, degradation commences when the ear tube is exposed to a solvent. The solvent is at least one of a glucose solution, hydrogen peroxide, carbamide peroxide, an alcohol, a calcium chelator, or EDTA.

In an example ear tube, the ear tube is a tympanostomy tube. In an example ear tube, the ear tube comprises a hollow cylindrical body having at least one flanged end. In an example ear tube, the ear tube includes a main tube, the main tube extending along a center axis defining a conduit for fluid communication, the main tube having a first end and a second end, and an outer flange extending radially away from the main tube on the first end. In an example ear tube, the ear tube includes a main tube, the main tube extending along a center axis defining a conduit for fluid communication, the main tube having a first end and a second end, an outer flange extending radially away from the main tube on a first end, an inner flange extending radially away from the main tube on a second end, and a frustoconical portion of the main tube extending longitudinally from at least one of the first end or the second end, the frustoconical portion extending beyond a width of the flange on the at least one of the first end or the second end.

Disclosed herein is an ear tube including chitosan assembled via an aqueous solution. The ear tube has at least one characteristic centrifugal property selected from the group consisting of i) a uniform density across at least a portion of the ear tube, ii) at least a portion of the ear tube is optically nontransparent, iii) at least a portion of the ear tube is free of exterior layering, iv) at least a portion of the ear tube is free of interior layering, v) at least a portion of the ear tube is interior-void-free, vi) at least a portion of the ear tube is free of cylindrical extrusion gaps, and vii) at least a portion of the ear tube is free of layering.

Disclosed herein is an ear tube including a hollow cylindrical body having at least one flanged end, wherein the ear tube includes chitosan, and the ear tube has at least one characteristic centrifugal property selected from the group consisting of: i) a uniform density across at least a portion of the ear tube, ii) at least a portion of the ear tube is optically nontransparent, iii) at least a portion of the ear tube is free of exterior layering, iv) at least a portion of the ear tube is free of interior layering, v) at least a portion of the ear tube is interior-void-free, vi) at least a portion of the ear tube is free of cylindrical extrusion gaps, and vii) at least a portion of the ear tube is free of layering.

Disclosed herein is an ear tube including a main tube, the main tube extending along a center axis defining a conduit for fluid communication, the main tube having a first end and a second end, and an outer flange extending radially away from the main tube on the first end, wherein the ear tube includes chitosan. The ear tube has at least one characteristic centrifugal property selected from the group consisting of: i) a uniform density across at least a portion of the ear tube, ii) at least a portion of the ear tube is optically nontransparent, iii) at least a portion of the ear tube is free of exterior layering, iv) at least a portion of the ear tube is free of interior layering, v) at least a portion of the ear tube is interior-void-free, vi) at least a portion of the ear tube is free of cylindrical extrusion gaps, and vii) at least a portion of the ear tube is free of layering.

Disclosed herein is an ear tube including a main tube, the main tube extending along a center axis defining a conduit for fluid communication, the main tube having a first end and a second end, an outer flange extending radially away from the main tube on a first end, an inner flange extending radially away from the main tube on a second end, and a frustoconical portion of the main tube extending longitudinally from at least one of the first end or the second end, the frustoconical portion extending beyond a width of the flange on the at least one of the first end or the second end, wherein the ear tube comprises chitosan. The ear tube has at least one characteristic centrifugal property selected from the group consisting of: i) a uniform density across at least a portion of the ear tube, ii) at least a portion of the ear tube is optically nontransparent, iii) at least a portion of the ear tube is free of exterior layering, iv) at least a portion of the ear tube is free of interior layering, v) at least a portion of the ear tube is interior-void-free, vi) at least a portion of the ear tube is free of cylindrical extrusion gaps, and vii) at least a portion of the ear tube is free of layering.

Disclosed herein is a method of forming an ear tube including depositing a chitosan solution into an ear tube mold, subjecting the ear tube mold and its contents to a first centrifugation, depositing a polymerization bath into the ear tube mold and subjecting the ear tube mold and its contents to a second centrifugation to polymerize the chitosan solution into a shape of an ear tube, and drying the ear tube.

In an example method, the ear tube dwells in the mold after the second centrifugation to complete polymerization before the drying.

An example method further includes subjecting the ear tube to at least one of cross-linking, dipcoating, covalent bonding, heat treatment, pressure treatment, water annealing, sterilization, surface modifications, chemical etching, chemical smoothing, or physical smoothing.

Disclosed herein is a method of degrading an ear tube including exposing an ear tube to a solvent, wherein the ear tube includes chitosan. The ear tube has at least one characteristic centrifugal property selected from the group consisting of: i) a uniform density across at least a portion of the ear tube, ii) at least a portion of the ear tube is optically nontransparent, iii) at least a portion of the ear tube is free of exterior layering, iv) at least a portion of the ear tube is free of interior layering, v) at least a portion of the ear tube is interior-void-free, vi) at least a portion of the ear tube is free of cylindrical extrusion gaps, and vii) at least a portion of the ear tube is free of layering.

In an example method, the solvent is at least one of a glucose solution, hydrogen peroxide, carbamide peroxide, an alcohol, a calcium chelator, or EDTA.

In an example method, exposing is over a period of at least one of minutes, hours, or days.

According to various embodiments, a variety of functionalizing agents may be used with the silk-containing embodiments described herein (e.g., silk membrane, silk composition, silk articles, silk matrix, silk foam, silk microsphere, liquid composition, whipped silk cream, silk meringue, compressed silk meringue, hot-pressed silk meringue, silk leather, silk powder, silk toner, etc.). It should be understood that the examples herein may recite one or a few silk-containing embodiments but are applicable to any silk-containing embodiment, as applicable. In some embodiments, a functionalizing agent may be any compound or molecule that facilitates the attachment to and/or development (e.g., growth) of one or more endothelial cells on a silk membrane. In some embodiments, a functionalizing agent may be any compound or molecule that facilitates the attachment and/or development (e.g., growth) of one or more megakaryocytes and/or hematopoietic progenitor cells on a silk matrix and/or silk membrane. In some embodiments, a functionalizing agent may be or comprise an agent suitable for facilitating the production of one or more of white blood cells and red blood cells.

In some embodiments, a functionalizing agent may be or comprise a cell attachment mediator and/or an extracellular matrix protein, for example: collagen (e.g., collagen type I, collagen type III, collagen type IV or collagen type VI), elastin, fibronectin, vitronectin, laminin, fibrinogen, von Willebrand factor, proteoglycans, decorin, perlecan, nidogen, hyaluronan, and/or peptides containing known integrin binding domains e.g. “RGD” integrin binding sequence, or variations thereof, that are known to affect cellular attachment.

In some embodiments, a functionalizing agent may be any soluble molecule produced by endothelial cells. Non-limiting examples include fibroblast growth factor-1 (FGF1) and vascular endothelial growth factors (VEGF).

According to some embodiments, a plurality of functionalizing agents may be used. For example, in some embodiments wherein production of platelets is desired, provided compositions may comprise the use of laminin, fibronectin and/or fibrinogen, and type IV collagen in order to facilitate the attachment and growth of endothelial cells on a silk membrane (e.g., a porous silk membrane) and/or attachment of megakaryocytes to a silk matrix.

In some embodiments, a functionalizing agent may be embedded or otherwise associated with a silk membrane and/or silk matrix such that at least a portion of the functionalizing agent is surrounded by a silk membrane and/or silk matrix as contrasted to a functionalizing agent simply being positioned along the surface of a silk membrane and/or silk matrix. In some embodiments, a functionalizing agent is distributed along and/or incorporated in substantially the entire surface area of a silk membrane/silk wall. In some embodiments, a functionalizing agent is distributed and/or incorporated only at one or more discrete portions of a silk membrane/wall and/or silk matrix. In some embodiments, a functionalizing agent is distributed in and/or along at least one of the lumen-facing side of a silk wall and the matrix-facing side of a silk wall.

According to various embodiments, any application-appropriate amount of one or more functionalizing agents may be used. In some embodiments, the amount of an individual functionalizing agent may be between about 1 μg/ml and 1,000 μg/ml (e.g., between about 2 and 1,000, 5 and 1,000, 10 and 1,000, 10 and 500, 10 and 100 μg/ml). In some embodiments, the amount of an individual functionalizing agent may be at least 1 μg/ml (e.g., at least 5, 10, 15, 20 25, 50, 100, 200, 300 400, 500, 600, 700, 800, or 900 μg/ml). In some embodiments, the amount of an individual functionalizing agent is at most 1,000 μg/ml (e.g., 900, 800, 700, 600, 500, 400, 300 200, 100, 90, 80, 70, 60, 50, 40, 30, 20, 10, or 5 μg/ml).

In some aspects, the composition comprises one or more sensing agents, such as a sensing dye. The sensing agents/sensing dyes are environmentally sensitive and produce a measurable response to one or more environmental factors. In some aspects, the environmentally-sensitive agent or dye may be present in the composition in an effective amount to alter the composition from a first chemical-physical state to a second chemical-physical state in response to an environmental parameter (e.g., a change in pH, light intensity or exposure, temperature, pressure or strain, voltage, physiological parameter of a subject, and/or concentration of chemical species in the surrounding environment) or an externally applied stimulus (e.g., optical interrogation, acoustic interrogation, and/or applied heat). In some cases, the sensing dye is present to provide one optical appearance under one given set of environmental conditions and a second, different optical appearance under a different given set of environmental conditions. Suitable concentrations for the sensing agents described herein can be the concentrations for the colorants and additives described elsewhere herein. A person having ordinary skill in the chemical sensing arts can determine a concentration that is appropriate for use in a sensing application of the inks described herein.

In some aspects, the first and second chemical-physical state may be a physical property of the composition, such as mechanical property, a chemical property, an acoustical property, an electrical property, a magnetic property, an optical property, a thermal property, a radiological property, or an organoleptic property. Exemplary sensing dyes or agents include, but are not limited to, a pH sensitive agent, a thermal sensitive agent, a pressure or strain sensitive agent, a light sensitive agent, or a potentiometric agent.

Exemplary pH sensitive dyes or agents include, but are not limited to, cresol red, methyl violet, crystal violet, ethyl violet, malachite green, methyl green, 2-(p-dimethylaminophenylazo) pyridine, paramethyl red, metanil yellow, 4-phenylazodiphenylamine, thymol blue, metacresol purple, orange IV, 4-o-Tolylazo-o-toluindine, quinaldine red, 2,4-dinitrophenol, erythrosine disodium salt, benzopurpurine 4B, N,N-dimethyl-p-(m-tolylazo) aniline, p-dimethylaminoazobenene, 4,4′-bis(2-amino-1-naphthylazo)-2,2′-stilbenedisulfonic acid, tetrabromophenolphthalein ethyl ester, bromophenol blue, Congo red, methyl orange, ethyl orange, 4-(4-dimethylamino-1-naphylazo)-3-methoxybenesulfonic acid, bromocresol green, resazurin, 4-phenylazo-1-napthylamine, ethyl red 2-(1-dimethylaminophenyazo) pyridine, 4-(p-ethoxypehnylazo)-m-phenylene-diamine monohydrochloride, resorcin blue, alizarin red S, methyl red, propyl red, bromocresol purple, chlorophenol red, p-nitrophenol, alizarin 2-(2,4-dinitrophenylazo) 1-napthol-3,6-disulfonic acid, bromothymol blue, 6,8-dinitro-2,4-(1H) quinazolinedione, brilliant yellow, phenol red, neutral red, m-nitrophenol, cresol red, turmeric, metacresol purple, 4,4′-bis(3-amino-1-naphthylazo)-2,2′-stilbenedisulfonic acid, thymol blue, p-naphtholbenzein, phenolphthalein, o-cresolphthalein, ethyl bis(2,4-dimethylphenyl) ethanoate, thymolphthalein, nitrazine yellow, alizarin yellow R, alizarin, p-(2,4-dihydroxyphenylazo) benzenesulfonic acid, 5,5′-indigodisulfonic acid, 2,4,6-trinitrotoluene, 1,3,5-trinitrobenezne, and clayton yellow.

Exemplary light responsive dyes or agents include, but are not limited to, photochromic compounds or agents, such as triarylmethanes, stilbenes, azasilbenes, nitrones, fulgides, spiropyrans, napthopyrans, spiro-oxzines, quinones, derivatives and combinations thereof.

Exemplary potentiometric dyes include, but are not limited to, substituted amiononaphthylehenylpridinium (ANEP) dyes, such as di-4-ANEPPS, di-8-ANEPPS, and N-(4-Sulfobutyl)-4-(6-(4-(Dibutylamino)phenyl) hexatrienyl) Pyridinium (RH237).

Exemplary temperature sensitive dyes or agents include, but are not limited to, thermochromic compounds or agents, such as thermochromic liquid crystals, leuco dyes, fluoran dyes, octadecylphosphonic acid.

Exemplary pressure or strain sensitive dyes or agents include, but are not limited to, spiropyran compounds and agents.

Exemplary chemi-sensitive dyes or agents include, but are not limited to, antibodies such as immunoglobulin G (IgG) which may change color from blue to red in response to bacterial contamination.

In some aspects, the compositions comprise one or more additive, dopant, or biologically active agent suitable for a desired intended purpose. In some aspects, the additive or dopant may be present in the composition in an amount effective to impart an optical or organoleptic property to the composition. Exemplary additives or dopants that impart optical or organoleptic properties include, but are not limited to, dyes/pigments, flavorants, aroma compounds, granular or fibrous fillers.

Additionally or alternatively, the additive, dopant, or biologically active agent may be present in the composition in an amount effective to “functionalize” the composition to impart a desired mechanical property or added functionality to the composition. Exemplary additive, dopants, or biologically active agent that impart the desired mechanical property or added functionality include, but are not limited to: environmentally sensitive/sensing dyes; active biomolecules; conductive or metallic particles; micro and nanofibers (e.g., silk nanofibers for reinforcement, carbon nanofibers); nanotubes; inorganic particles (e.g., hydroxyapatite, tricalcium phosphate, bioglasses); drugs (e.g., antibiotics, small molecules or low molecular weight organic compounds); proteins and fragments or complexes thereof (e.g., enzymes, antigens, antibodies and antigen-binding fragments thereof); DNA/RNA (e.g., siRNA, miRNA, mRNA); cells and fractions thereof (viruses and viral particles; prokaryotic cells such as bacteria; eukaryotic cells such as mammalian cells and plant cells; fungi).

In some aspects, the additive or dopant comprises a flavoring agent or flavorant.

Exemplary flavorants include ester flavorants, amino acid flavorants, nucleic acid flavorants, organic acid flavorants, and inorganic acid flavorants, such as, but not limited to, diacetyl, acetylpropionyl, acetoin, isoamyl acetate, benzaldehyde, cinnamaldehyde, ethyl propionate, methyl anthranilate, limonene, ethyl decadienoate, allyl hexanoate, ethyl maltol, ethylvanillin, methyl salicylate, manzanate, glutamic acid salts, glycine salts, guanylic acids salts, inosinic acid salts, acetic acid, ascorbic acid, citric acid, fumaric acid, lactic acid, malic acid, phosphoric acid, tartaric acid, derivatives, and mixtures thereof.

In some aspects, the additive or dopant comprises an aroma compound. Exemplary aroma compounds include ester aroma compounds, terpene aroma compounds, cyclic terpenes, and aromatic aroma compounds, such as, but not limited to, geranyl acetate, methyl formate, methyl acetate, methyl propionate, methyl butyrate, ethyl acetate, ethyl butyrate, isoamyl acetate, pentyl butrate, pentyl pentanoate, octyl acetate, benzyl acetate, methyl anthranilate, myrecene, geraniol, nerol, citral, cironellal, cironellol, linalool, nerolidol, limonene, camphor, menthol, carone, terpineol, alpha-lonone, thujone, eucalyptol, benzaldehyde, eugenol, cinnamaldehyde, ethyl maltol, vanillin, anisole, anethole, estragole, thymol.

In some aspects, the additive or dopant comprises a colorant, such as a dye or pigment. In some aspects, the dye or pigment imparts a color or grayscale to the composition. The colorant can be different than the sensing agents and/or sensing dyes below. Any organic and/or inorganic pigments and dyes can be included in the inks. Exemplary pigments suitable for use in the present disclosure include International Color Index or C.I. Pigment Black Numbers 1, 7, 1 1 and 31, C.I. Pigment Blue Numbers 15, 15:1, 15:2, 15:3, 15:4, 15:6, 16, 27, 29, 61 and 62, C.I. Pigment Green Numbers 7, 17, 18 and 36, C.I. Pigment Orange Numbers 5, 13, 16, 34 and 36, C.I. Pigment Violet Numbers 3, 19, 23 and 27, C.I. Pigment Red Numbers 3, 17, 22, 23, 48:1, 48:2, 57:1, 81:1, 81:2, 81:3, 81:5, 101, 1 14, 122, 144, 146, 170, 176, 179, 181, 185, 188, 202, 206, 207, 210 and 249, C.I. Pigment Yellow Numbers 1, 2, 3, 12, 13, 14, 17, 42, 65, 73, 74, 75, 83, 30, 93, 109, 1 10, 128, 138, 139, 147, 142, 151, 154 and 180, D&C Red No. 7, D&C Red No. 6 and D&C Red No. 34, carbon black pigment (such as Regal 330, Cabot Corporation), quinacridone pigments (Quinacridone Magenta (228-0122), available from Sun Chemical Corporation, Fort Lee, N.J.), diarylide yellow pigment (such as AAOT Yellow (274-1788) available from Sun Chemical Corporation); and phthalocyanine blue pigment (such as Blue 15:3 (294-1298) available from Sun Chemical Corporation). The classes of dyes suitable for use in present invention can be selected from acid dyes, natural dyes, direct dyes (either cationic or anionic), basic dyes, and reactive dyes. The acid dyes, also regarded as anionic dyes, are soluble in water and mainly insoluble in organic solvents and are selected, from yellow acid dyes, orange acid dyes, red acid dyes, violet acid dyes, blue acid dyes, green acid dyes, and black acid dyes. European Patent 0745651, incorporated herein by reference, describes a number of acid dyes that are suitable for use in the present disclosure. Exemplary yellow acid dyes include Acid Yellow 1 International Color Index or C.I. 10316); Acid Yellow 7 (C.I. 56295); Acid Yellow 17 (C.I. 18965); Acid Yellow 23 (C.I. 19140); Acid Yellow 29 (C.I. 18900); Acid Yellow 36 (C.I. 13065); Acid Yellow 42 (C.I. 22910); Acid Yellow 73 (C.I. 45350); Acid Yellow 99 (C.I. 13908); Acid Yellow 194; and Food Yellow 3 (C.I. 15985). Exemplary orange acid dyes include Acid Orange 1 (C.I. 13090/1); Acid Orange 10 (C.I. 16230); Acid Orange 20 (C.I. 14603); Acid Orange 76 (C.I. 18870); Acid Orange 142; Food Orange 2 (C.I. 15980); and Orange B.

Exemplary red acid dyes include Acid Red 1. (C.I. 18050); Acid Red 4 (C.I. 14710); Acid Red 18 (C.I. 16255), Acid Red 26 (C.I. 16150); Acid Red 2.7 (C.I. as Acid Red 51 (C.I. 45430, available from BASF Corporation, Mt. Olive, N.J.) Acid Red 52 (C.I. 45100); Acid Red 73 (C.I. 27290); Acid Red 87 (C. I. 45380); Acid Red 94 (C.I. 45440) Acid Red 194; and Food Red 1 (C.I. 14700). Exemplary violet acid dyes include Acid Violet 7 (C.I. 18055); and Acid Violet 49 (C.I. 42640). Exemplary blue acid dyes include Acid Blue 1 (C.I. 42045); Acid Blue 9 (C.I. 42090); Acid Blue 22 (C.I. 42755); Acid Blue 74 (C.I. 73015); Acid Blue 93 (C.I. 42780); and Acid Blue 158A (C.I. 15050). Exemplary green acid dyes include Acid Green 1 (C.I. 10028); Acid Green 3 (C.I. 42085); Acid Green 5 (C.I. 42095); Acid Green 26 (C.I. 44025); and Food Green 3 (C.I. 42053). Exemplary black acid dyes include Acid Black 1 (C.I. 20470); Acid Black 194 (Basantol® X80, available from BASF Corporation, an azo/l: 2 CR-complex.

Exemplary direct dyes for use in the present disclosure include Direct Blue 86 (C.I. 74180); Direct Blue 199; Direct Black 168; Direct Red 253; and Direct Yellow 107/132 (C.I. Not Assigned).

Exemplary natural dyes for use in the present disclosure include Alkanet (C.I. 75520, 75530); Annafto (C.I. 75120); Carotene (C.I. 75130); Chestnut; Cochineal (C.I.75470); Cutch (C.I. 75250, 75260); Divi-Divi; Fustic (C.I. 75240); Hypernic (C.I. 75280); Logwood (C.I. 75200); Osage Orange (C.I. 75660); Paprika; Quercitron (C.I. 75720); Sanrou (C.I. 75100); Sandal Wood (C.I. 75510, 75540, 75550, 75560); Sumac; and Tumeric (C.I. 75300). Exemplary reactive dyes for use in the present disclosure include Reactive Yellow 37 (monoazo dye); Reactive Black 31 (disazo dye); Reactive Blue 77 (phthalo cyanine dye) and Reactive Red 180 and Reactive Red 108 dyes. Suitable also are the colorants described in The Printing Ink Manual (5th ed., Leach et al. eds. (2007), pages 289-299. Other organic and inorganic pigments and dyes and combinations thereof can be used to achieve the colors desired.

In addition to or in place of visible colorants, compositions provided herein can contain ETV fluorophores that are excited in the ETV range and emit light at a higher wavelength (typically 400 nm and above). Examples of ETV fluorophores include but are not limited to materials from the coumarin, benzoxazole, rhodamine, napthalimide, perylene, benzanthrones, benzoxanthones or benzothia-xanthones families. The addition of a UV fluorophore (such as an optical brightener for instance) can help maintain maximum visible light transmission. The amount of colorant, when present, generally is between 0.05% to 5% or between 0.1% and 1% based on the weight of the composition.

For non-white compositions, the amount of pigment/dye generally is present in an amount of from at or about 0.1 wt % to at or about 20 wt % based on the weight of the composition. In some applications, a non-white ink can include 15 wt % or less pigment/dye, or 10 wt % or less pigment/dye or 5 wt % pigment/dye, or 1 wt % pigment/dye based on the weight of the composition. In some applications, a non-white ink can include 1 wt % to 10 wt %, or 5 wt % to 15 wt %, or 10 wt % to 20 wt % pigment/dye based on the weight of the composition. In some applications, a non-white ink can contain an amount of dye/pigment that is 1 wt %, 2 wt %, 3 wt %, 4 wt %, 5%, 6 wt %, 7 wt %, 8 wt %, 9 wt %, 10 wt %, 11 wt %, 12 wt %, 13 wt %, 14 wt %, 15%, 16 wt %, 17 wt %, 18 wt %, 19 wt % or 20 wt % based on the weight of the composition.

For white compositions, the amount of white pigment generally is present in an amount of from at or about 1 wt % to at or about 60 wt % based on the weight of the composition. In some applications, greater than 60 wt % white pigment can be present. Preferred white pigments include titanium dioxide (anatase and rutile), zinc oxide, lithopone (calcined coprecipitate of barium sulfate and zinc sulfide), zinc sulfide, blanc fixe and alumina hydrate and combinations thereof, although any of these can be combined with calcium carbonate. In some applications, a white ink can include 60 wt % or less white pigment, or 55 wt % or less white pigment, or 50 wt % white pigment, or 45 wt % white pigment, or 40 wt % white pigment, or 35 wt % white pigment, or 30 wt % white pigment, or 25 wt % white pigment, or 20 wt % white pigment, or 15 wt % white pigment, or 10 wt % white pigment, based on the weight of the composition. In some applications, a white ink can include 5 wt % to 60 wt %, or 5 wt % to 55 wt %, or 10 wt % to 50 wt %, or 10 wt % to 25 wt %, or 25 wt % to 50 wt %, or 5 wt % to 15 wt %, or 40 wt % to 60 wt % white pigment based on the weight of the composition. In some applications, a non-white ink can an amount of dye/pigment that is 5%, 6 wt %, 7 wt %, 8 wt %, 9 wt %, 10 wt %, 11 wt %, 12 wt %, 13 wt %, 14 wt %, 15%, 16 wt %, 17 wt %, 18 wt %, 19 wt %, 20 wt %, 21 wt %, 22 wt %, 23 wt %, 24 wt %, 25%, 26 wt %, 27 wt %, 28 wt %, 29 wt %, 30 wt %, 31 wt %, 32 wt %, 33 wt %, 34 wt %, 35%, 36 wt %, 37 wt %, 38 wt %, 39 wt %, 40 wt %, 41 wt %, 42 wt %, 43 wt %, 44 wt %, 45%, 46 wt %, 47 wt %, 48 wt %, 49 wt %, 50 wt %, 51 wt %, 52 wt %, 53 wt %, 54 wt %, 55%, 56 wt %, 57 wt %, 58 wt %, 59 wt % or 60 wt % based on the weight of the composition.

In some aspects, the additive or dopant comprises a conductive additive. Exemplary conductive additives include, but are not limited to graphite, graphite powder, carbon nanotubes, and metallic particles or nanoparticles, such as gold nanoparticles. In some aspects, the conductive additive is biocompatible and non-toxic.

In some aspects, the additive is a biologically active agent. The term “biologically active agent” as used herein refers to any molecule which exerts at least one biological effect in vivo. For example, the biologically active agent can be a therapeutic agent to treat or prevent a disease state or condition in a subject. Biologically active agents include, without limitation, organic molecules, inorganic materials, proteins, peptides, nucleic acids (e.g., genes, gene fragments, gene regulatory sequences, and antisense molecules), nucleoproteins, polysaccharides, glycoproteins, and lipoproteins. Classes of biologically active compounds that can be incorporated into the composition provided herein include, without limitation, anticancer agents, antibiotics, analgesics, anti-inflammatory agents, immunosuppressants, enzyme inhibitors, antihistamines, anti-convulsants, hormones, muscle relaxants, antispasmodics, ophthalmic agents, prostaglandins, anti-depressants, anti-psychotic substances, trophic factors, osteoinductive proteins, growth factors, and vaccines.

The term “active agent” may also be used herein to refer to a biological sample (e.g., a sample of tissue or fluid, such as for instance blood) or a component thereof, and/or to a biologically active entity or compound, and/or to a structurally or functionally labile entity.

Exemplary active agents include, but are not limited to, therapeutic agents, diagnostic agents (e.g., contrast agents), and any combinations thereof. In some embodiments, the active agent present in a silk matrix (e.g., a silk microsphere), composition, or the like can include a labile active agent, e.g., an agent that can undergo chemical, physical, or biological change, degradation and/or deactivation after exposure to a specified condition, e.g., high temperatures, high humidity, light exposure, and any combinations thereof. In some embodiments, the active agent present in the silk matrix (e.g., a silk microsphere), composition, or the like can include a temperature-sensitive active agent, e.g., an active agent that will lose at least about 30% or more, of its original activity or bioactivity, upon exposure to a temperature of at least about 10° C. or above, including at least about 15° C. or above, at least about room temperature or above, or at least about body temperature (e.g., about 37° C.) or above.

The active agent can be generally present in the silk matrix (e.g., a silk microsphere), composition, or the like in an amount of about 0.01% (w/w) to about 70% (w/w), or about 0.1% (w/w) to about 50% (w/w), or about 1% (w/w) to about 30% (w/w). The active agent can be present on a surface of the silk matrix (e.g., a silk microsphere), composition, or the like and/or encapsulated and dispersed in the silk matrix (e.g., a silk microsphere), composition, or the like homogeneously or heterogeneously or in a gradient. In some embodiments, the active agent can be added into the silk solution, which is then subjected to the methods described herein for preparing a silk matrix (e.g., a silk microsphere), composition, or the like. In some embodiments, the active agent can be coated on a surface of the silk matrix (e.g., a silk microsphere), composition, or the like. In some embodiments, the active agent can be loaded in a silk matrix (e.g., a silk microsphere), composition, or the like by incubating the silk microsphere in a solution of the active agent for a period of time, during which an amount of the active agent can diffuse into the silk matrix (e.g., a silk microsphere), composition, or the like, and thus distribute within the silk matrix (e.g., a silk microsphere), composition, or the like.

In some aspects, the additive is a therapeutic agent. As used herein, the term “therapeutic agent” means a molecule, group of molecules, complex or substance administered to an organism for diagnostic, therapeutic, preventative medical, or veterinary purposes. As used herein, the term “therapeutic agent” includes a “drug” or a “vaccine.” This term include externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term can also be used in reference to agricultural, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a therapeutic effect, for example deoxyribonucleic acid (DNA), ribonucleic acid (RNA), nucleic acid analogues (e.g., locked nucleic acid (LNA), peptide nucleic acid (PNA), xeno nucleic acid (XNA)), or mixtures or combinations thereof, including, for example, DNA nanoplexes, siRNA, microRNA, shRNA, aptamers, ribozymes, decoy nucleic acids, antisense nucleic acids, RNA activators, and the like. Generally, any therapeutic agent can be included in the composition provided herein.

The term “therapeutic agent” also includes an agent that is capable of providing a local or systemic biological, physiological, or therapeutic effect in the biological system to which it is applied. For example, the therapeutic agent can act to control infection or inflammation, enhance cell growth and tissue regeneration, control tumor growth, act as an analgesic, promote anti-cell attachment, and enhance bone growth, among other functions. Other suitable therapeutic agents can include anti-viral agents, hormones, antibodies, or therapeutic proteins. Other therapeutic agents include prodrugs, which are agents that are not biologically active when administered but, upon administration to a subject are converted to biologically active agents through metabolism or some other mechanism. Additionally, a silk-based drug delivery composition can contain one therapeutic agent or combinations of two or more therapeutic agents.

A therapeutic agent can include a wide variety of different compounds, including chemical compounds and mixtures of chemical compounds, e.g., small organic or inorganic molecules; saccharines; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; an extract made from biological materials such as bacteria, plants, fungi, or animal cells; animal tissues; naturally occurring or synthetic compositions; and any combinations thereof. In some aspects, the therapeutic agent is a small molecule.

The term “bioactivity,” as used herein in reference to an active agent, generally refers to the ability of an active agent to interact with a biological target and/or to produce an effect on a biological target. For example, bioactivity can include, without limitation, elicitation of a stimulatory, inhibitory, regulatory, toxic or lethal response in a biological target. The biological target can be a molecule or a cell. For example, a bioactivity can refer to the ability of an active agent to modulate the effect/activity of an enzyme, block a receptor, stimulate a receptor, modulate the expression level of one or more genes, modulate cell proliferation, modulate cell division, modulate cell morphology, or any combination thereof. In some instances, a bioactivity can refer to the ability of a compound to produce a toxic effect in a cell. Exemplary cellular responses include, but are not limited to, lysis, apoptosis, growth inhibition, and growth promotion; production, secretion, and surface expression of a protein or other molecule of interest by the cell; membrane surface molecule activation including receptor activation; transmembrane ion transports; transcriptional regulations; changes in viability of the cell; changes in cell morphology; changes in presence or expression of an intracellular component of the cell; changes in gene expression or transcripts; changes in the activity of an enzyme produced within the cell; and changes in the presence or expression of a ligand and/or receptor (e.g., protein expression and/or binding activity). Methods for assaying different cellular responses are well known to one of skill in the art, e.g., western blot for determining changes in presence or expression of an endogenous protein of the cell, or microscopy for monitoring the cell morphology in response to the active agent, or FISH and/or qPCR for the detection and quantification of changes in nucleic acids. Bioactivity can be determined in some embodiments, for example, by assaying a cellular response.

In reference to an antibody, the term “bioactivity” includes, but is not limited to, epitope or antigen binding affinity, the in vivo and/or in vitro stability of the antibody, the immunogenic properties of the antibody, e.g., when administered to a human subject, and/or the ability to neutralize or antagonize the bioactivity of a target molecule in vivo or in vitro. The aforementioned properties or characteristics can be observed or measured using art-recognized techniques including, but not limited to, scintillation proximity assays, ELISA, ORIGEN immunoassay (IGEN), fluorescence quenching, fluorescence ELISA, competitive ELISA, SPR analysis including, but not limited to, SPR analysis using a BIAcore biosensor, in vitro and in vivo neutralization assays (see, for example, International Publication No. WO 2006/062685), receptor binding, and immunohistochemistry with tissue sections from different sources including human, primate, or any other source as needed. In reference to an immunogen, the “bioactivity” includes immunogenicity, the definition of which is discussed in detail later. In reference to a virus, the “bioactivity” includes infectivity, the definition of which is discussed in detail later. In reference to a contrast agent, e.g., a dye, the “bioactivity” refers to the ability of a contrast agent when administered to a subject to enhance the contrast of structures or fluids within the subject's body. The bioactivity of a contrast agent also includes, but is not limited to, its ability to interact with a biological environment and/or influence the response of another molecule under certain conditions.

As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight of less than 5000 Daltons (5 kDa), preferably less than 3 kDa, still more preferably less than 2 kDa, and most preferably less than 1 kDa. In some cases it is preferred that a small molecule have a molecular weight equal to or less than 700 Daltons.

Exemplary therapeutic agents include, but are not limited to, those found in Harrison's Principles of Internal Medicine, 13th Edition, Eds. T. R. Harrison et al. McGraw-Hill N.Y., NY; Physicians' Desk Reference, 50th Edition, 1997, Oradell New Jersey, Medical Economics Co.; Pharmacological Basis of Therapeutics, 8th Edition, Goodman and Gilman, 1990; United States Pharmacopeia, The National Formulary, ETSP XII NF XVII, 1990, the complete contents of all of which are incorporated herein by reference.

Therapeutic agents include the herein disclosed categories and specific examples. It is not intended that the category be limited by the specific examples. Those of ordinary skill in the art will recognize also numerous other compounds that fall within the categories and that are useful according to the present disclosure. Examples include a radiosensitizer, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, an anticholinergic/antispasmodic agent, a vasopressin analogue, an anti arrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, a vaccine, a protein, or a nucleic acid. In a further aspect, the pharmaceutically active agent can be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenoterol, clenbuterol, bambuterol, salmeterol, fenoterol; antiinflammatory agents, including antiasthmatic anti-inflammatory agents, antiarthritics antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetaminophen, ibuprofen, ketoprofen and piroxicam; analgesic agents such as salicylates; calcium channel blockers such as nifedipine, amlodipine, and nicardipine; angiotensin converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists such as clonidine; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; antiarrhythmic agents such as quinidine, lidocaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as Coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hydrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides.

Anti-cancer agents include alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists/antagonists, endothelin A receptor antagonists, retinoic acid receptor agonists, immuno-modulators, hormonal and antihormonal agents, photodynamic agents, and tyrosine kinase inhibitors.

Antibiotics include aminoglycosides (e.g., gentamicin, tobramycin, netilmicin, streptomycin, amikacin, neomycin), bacitracin, corbapenems (e.g., imipenem/cislastatin), cephalosporins, colistin, methenamine, monobactams (e.g., aztreonam), penicillins (e.g., penicillin G, penicillin V, methicillin, natacillin, oxacillin, cloxacillin, dicloxacillin, ampicillin, amoxicillin, carbenicillin, ticarcillin, piperacillin, mezlocillin, azlocillin), polymyxin B, quinolones, and vancomycin; and bacteriostatic agents such as chloramphenicol, clindanyan, macrolides (e.g., erythromycin, azithromycin, clarithromycin), lincomyan, nitrofurantoin, sulfonamides, tetracyclines (e.g., tetracycline, doxycycline, minocycline, demeclocycline), and trimethoprim. Also included are metronidazole, fluoroquinolones, and ritampin.

Enzyme inhibitors are substances which inhibit an enzymatic reaction. Examples of enzyme inhibitors include edrophonium chloride, N-methylphysostigmine, neostigmine bromide, physostigmine sulfate, tacrine, tacrine, 1-hydroxy maleate, iodotubercidin, p-bromotetramiisole, 10-(alpha-diethylaminopropionyl)-phenothiazine hydrochloride, calmidazolium chloride, hemicholinium-3,3,5-dinitrocatechol, diacylglycerol kinase inhibitor I, diacylglycerol kinase inhibitor II, 3-phenylpropargylamine, N°-monomethyl-Larginine acetate, carbidopa, 3-hydroxybenzylhydrazine, hydralazine, clorgyline, deprenyl, hydroxylamine, iproniazid phosphate, 6-MeO-tetrahydro-9H-pyrido-indole, nialamide, pargyline, quinacrine, semi carb azide, tranylcypromine, N,N-diethylaminoethyl-2,2-diphenylvalerate hydrochloride, 3-isobutyl-1-methylxanthne, papaverine, indomethacind, 2-cyclooctyl-2-hydroxy ethylamine hydrochloride, 2,3-dichloro-a-methylbenzylamine (DCMB), 8,9-dichloro-2,3,4,5-tetrahydro-1H-2-benzazepine hydrochloride, p-amino glutethimide, p-aminoglutethimide tartrate, 3-iodotyrosine, alpha-methyltyrosine, acetazolamide, dichlorphenamide, 6-hydroxy-2-benzothiazolesulfonamide, and allopurinol.

Antihistamines include pyrilamine, chlorpheniramine, and tetrahydrazoline, among others.

Anti-inflammatory agents include corticosteroids, nonsteroidal anti-inflammatory drugs (e.g., aspirin, phenylbutazone, indomethacin, sulindac, tolmetin, ibuprofen, piroxicam, and fenamates), acetaminophen, phenacetin, gold salts, chloroquine, D-Penicillamine, methotrexate colchicine, allopurinol, probenecid, and sulfinpyrazone.

Muscle relaxants include mephenesin, methocarbamol, cyclobenzaprine hydrochloride, trihexylphenidyl hydrochloride, levodopa/carbidopa, and biperiden.

Anti-spasmodics include atropine, scopolamine, oxyphenonium, and papaverine.

Analgesics include aspirin, phenybutazone, idomethacin, sulindac, tolmetic, ibuprofen, piroxicam, fenamates, acetaminophen, phenacetin, morphine sulfate, codeine sulfate, meperidine, nalorphine, opioids (e.g., codeine sulfate, fentanyl citrate, hydrocodone bitartrate, loperamide, morphine sulfate, noscapine, norcodeine, normorphine, thebaine, nor-binaltorphimine, buprenorphine, chlomaltrexamine, funaltrexamione, nalbuphine, nalorphine, naloxone, naloxonazine, naltrexone, and naltrindole), procaine, lidocain, tetracaine and dibucaine. Ophthalmic agents include sodium fluorescein, rose bengal, methacholine, adrenaline, cocaine, atropine, alpha-chymotrypsin, hyaluronidase, betaxalol, pilocarpine, timolol, timolol salts, and combinations thereof.

Prostaglandins are art recognized and are a class of naturally occurring chemically related long-chain hydroxy fatty acids that have a variety of biological effects.

Anti-depressants are substances capable of preventing or relieving depression.

Examples of anti-depressants include imipramine, amitriptyline, nortriptyline, protriptyline, desipramine, amoxapine, doxepin, maprotiline, tranylcypromine, phenelzine, and isocarboxazide.

Trophic factors are factors whose continued presence improves the viability or longevity of a cell trophic factors include, without limitation, platelet-derived growth factor (PDGP), neutrophil-activating protein, monocyte chemoattractant protein, macrophage-inflammatory protein, platelet factor, platelet basic protein, and melanoma growth stimulating activity; epidermal growth factor, transforming growth factor (alpha), fibroblast growth factor, platelet-derived endothelial cell growth factor, insulin-like growth factor, glial derived growth neurotrophic factor, ciliary neurotrophic factor, nerve growth factor, bone growth/cartilage-inducing factor (alpha and beta), bone morphogenetic proteins, interleukins (e.g., interleukin inhibitors or interleukin receptors, including interleukin 1 through interleukin 10), interferons (e.g., interferon alpha, beta and gamma), hematopoietic factors, including erythropoietin, granulocyte colony stimulating factor, macrophage colony stimulating factor and granulocyte-macrophage colony stimulating factor; tumor necrosis factors, and transforming growth factors (beta), including beta-1, beta-2, beta-3, inhibin, and activin.

Hormones include estrogens (e.g., estradiol, estrone, estriol, diethylstibestrol, quinestrol, chlorotrianisene, ethinyl estradiol, mestranol), anti-estrogens (e.g., clomiphene, tamoxifen), progestins (e.g., medroxyprogesterone, norethindrone, hydroxyprogesterone, norgestrel), antiprogestin (mifepristone), androgens (e.g, testosterone cypionate, fluoxymesterone, danazol, testolactone), anti-androgens (e.g., cyproterone acetate, flutamide), thyroid hormones (e.g., triiodothyronne, thyroxine, propylthiouracil, methimazole, and iodixode), and pituitary hormones (e.g., corticotropin, sumutotropin, oxytocin, and vasopressin). Hormones are commonly employed in hormone replacement therapy and/or for purposes of birth control. Steroid hormones, such as prednisone, are also used as immunosuppressants and anti-inflammatories. In some aspects, the additive is an agent that stimulates tissue formation, and/or healing and regrowth of natural tissues, and any combinations thereof. Agents that increase formation of new tissues and/or stimulates healing or regrowth of native tissue at the site of injection can include, but are not limited to, fibroblast growth factor (FGF), transforming growth factor-beta (TGF-beta, platelet-derived growth factor (PDGF), epidermal growth factors (EGFs), connective tissue activated peptides (CTAPs), osteogenic factors including bone morphogenic proteins, heparin, angiotensin II (A-II) and fragments thereof, insulin-like growth factors, tumor necrosis factors, interleukins, colony stimulating factors, erythropoietin, nerve growth factors, interferons, biologically active analogs, fragments, and derivatives of such growth factors, and any combinations thereof.

In some aspects, the silk composition can further comprise at least one additional material for soft tissue augmentation, e.g., dermal filler materials, including, but not limited to, poly(methyl methacrylate) microspheres, hydroxylapatite, poly(L-lactic acid), collagen, elastin, and glycosaminoglycans, hyaluronic acid, commercial dermal filler products such as BOTOX® (from Allergan), DYSPORT®, COSMODERM®, EVOLENCE®, RADIESSE®, RESTYLANE®, JUVEDERM® (from Allergan), SCULPTRA®, PERLANE®, and CAPTIQEIE®, and any combinations thereof.

In some aspects, the additive is a wound healing agent. As used herein, a “wound healing agent” is a compound or composition that actively promotes wound healing process.

Exemplary wound healing agents include, but are not limited to dexpanthenol; growth factors; enzymes, hormones; povidon-iodide; fatty acids; anti-inflammatory agents; antibiotics; antimicrobials; antiseptics; cytokines; thrombin; analgesics; opioids; aminoxyls; furoxans; nitrosothiols; nitrates and anthocyanins; nucleosides, such as adenosine; and nucleotides, such as adenosine diphosphate (ADP) and adenosine triphosphate (ATP); neurotransmitter/neuromodulators, such as acetylcholine and 5-hydroxytryptamine (serotonin/5-HT); histamine and catecholamines, such as adrenalin and noradrenalin; lipid molecules, such as 5 sphingosine-1-phosphate and lysophosphatidic acid; amino acids, such as arginine and lysine; peptides such as the bradykinins, substance P and calcium gene-related peptide (CGRP); nitric oxide; and any combinations thereof.

In certain aspects, the active agents provided herein are immunogens. In one aspect, the immunogen is a vaccine. Most vaccines are sensitive to environmental conditions under which they are stored and/or transported. For example, freezing may increase reactogenicity (e.g., capability of causing an immunological reaction) and/or loss of potency for some vaccines (e.g., HepB, and DTaP/IPV/FQB), or cause hairline cracks in the container, leading to contamination. Further, some vaccines (e.g., BCG, Varicella, and MMR) are sensitive to heat. Many vaccines (e.g., BCG, MMR, Varicella, Meningococcal C Conjugate, and most DTaP-containing vaccines) are light sensitive. See, e.g., Galazka et al., Thermostability of vaccines, in Global Programme for Vaccines & Immunization (World Health Organization, Geneva, 1998); Peetermans et al., Stability of freeze-dried rubella virus vaccine (Cendehill strain) at various temperatures, 1 J. Biological Standardization 179 (1973). Thus, the compositions and methods provided herein also provide for stabilization of vaccines regardless of the cold chain and/or other environmental conditions.

In some aspects, the additive is a cell, e.g., a biological cell. Cells useful for incorporation into the composition can come from any source, e.g., mammalian, insect, plant, etc. In some aspects, the cell can be a human, rat or mouse cell. In general, cells to be used with the compositions provided herein can be any types of cells. In general, the cells should be viable when encapsulated within compositions. In some aspects, cells that can be used with the composition include, but are not limited to, mammalian cells (e.g. human cells, primate cells, mammalian cells, rodent cells, etc.), avian cells, fish cells, insect cells, plant cells, fungal cells, spore cells, bacterial cells, and hybrid cells. In some aspects, exemplary cells that can be can be used with the compositions include platelets, activated platelets, stem cells, totipotent cells, pluripotent cells, and/or embryonic stem cells. In some aspects, exemplary cells that can be encapsulated within compositions include, but are not limited to, primary cells and/or cell lines from any tissue. For example, cardiomyocytes, myocytes, hepatocytes, keratinocytes, melanocytes, neurons, astrocytes, embryonic stem cells, adult stem cells, hematopoietic stem cells, hematopoietic cells (e.g. monocytes, neutrophils, macrophages, etc.), ameloblasts, fibroblasts, chondrocytes, osteoblasts, osteoclasts, neurons, sperm cells, egg cells, liver cells, epithelial cells from lung, epithelial cells from gut, epithelial cells from intestine, liver, epithelial cells from skin, etc., and/or hybrids thereof, can be included in the silk/platelet compositions disclosed herein. Those skilled in the art will recognize that the cells listed herein represent an exemplary, not comprehensive, list of cells. Cells can be obtained from donors (allogenic) or from recipients (autologous). Cells can be obtained, as a non-limiting example, by biopsy or other surgical means known to those skilled in the art.

In some aspects, the cell can be a genetically modified cell. A cell can be genetically modified to express and secrete a desired compound, e.g. a bioactive agent, a growth factor, differentiation factor, cytokines, and the like. Methods of genetically modifying cells for expressing and secreting compounds of interest are known in the art and easily adaptable by one of skill in the art.

Differentiated cells that have been reprogrammed into stem cells can also be used.

For example, human skin cells reprogrammed into embryonic stem cells by the transduction of Oct3/4, Sox2, c-Myc and Klf4 (Junying Yu, et. ah, Science, 2007, 318, 1917-1920 and Takahashi K. et. ah, Cell, 2007, 131, 1-12).

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

While the invention has been illustrated and described in detail in the foregoing drawings and description, the same is to be considered as illustrative and not restrictive in character, it being understood that only illustrative embodiments thereof have been shown and described and that all changes and modifications that come within the spirit of the invention are desired to be protected. For example, any of the features or functions of any of the embodiments disclosed herein may be incorporated into any of the other embodiments disclosed herein.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

### Example 1

Disclosed herein is the bioengineering of next generation tympanostomy tubes using degradable natural biopolymers (e.g., silk fibroin protein and chitosan polysaccharide) as a suitable alternative to existing nondegradable commercially available ear tubes and manufacturing methods for fabricating biomaterial-based ear tubes with the size, geometry, and mechanical properties similar to existing commercial products. The disclosed ear tubes incorporate enhanced function through local elution of antibiotics and antiinflammatory agents to prevent post-operative infections while also degrading on-demand (e.g., Tunable degradation rate (1-3 days), non-toxic byproducts) without surgical intervention. The biocompatibility of the ear tubes is evaluated both in vitro and in vivo using rodent models to determine the impact of enhanced functionality on hearing, local tympanic membrane tissue effects, and degradability of ear tubes in comparison to non-functionalized or commercial products.

Commercially available, non-degradable ear tubes for treating middle ear infection suffer from inconsistencies in performance including falling out too early, becoming stuck in the ear drum, and clogging to prevent fluid drainage. Ear tubes disclosed herein have appropriate mechanical properties for easy handling during operation procedures, elution of antibiotics/steroids post-operation, maintenance of patency for the duration of implantation, and unique on demand dissolution of the tube at completion.

Tympanostomy tubes are commonly used to improve middle ear ventilation for ear infections that do not resolve on their own, with standard treatments (e.g., antibiotics), or for children predisposed to recurring ear infections. Current commercially available tubes are made of synthetic, non-degradable materials that are designed to self extrude but can be unpredictable, either prematurely falling out or becoming stuck and requiring surgical removal. These tubes can also become clogged or coated with biofilm, thus losing their ability to drain the middle ear and requiring administration of antibiotics to resolve this issue. While there have been significant attempts at developing ear tubes with desirable compatibility and resorption rates, there are still major limitations facing past and present approaches. An ideal ear tube would be biocompatible, have predictable and on demand resorption rates, have added benefits of serving as a drug eluting device, and mechanical properties that are well-suited to implantation in the tympanic membrane (TM). One of the main drawbacks apparent from commercially available tubes is that following placement, patients may experience discharge from the ear (post-tube otorrhea) which is frequently the result of biofilm formation. Such local infections can lead to mild to moderate TM inflammatory responses which, in turn, increases the risk of downstream tube retention and TM perforations. Therefore, to address these limitations, it is necessary to design a device that is both mechanically and biologically compatible, resorbable, and with drug eluting capabilities. In the present design, silk fibroin and chitosan biomaterials are used to create ear tubes out of materials with both robust and tailorable mechanical properties, as well as degradation capabilities and biocompatibility. A reduced inflammatory response associated with the silk/chitosan tubes will eliminate the possibility of local changes of the ear drum, or any other local biological tissue of the middle ear pathway that may result in long-term hearing loss and/or cochlear dysfunctions. Furthermore, given the all-aqueous processing methods of silk/chitosan, these devices can be functionalized with antibiotics and anti-inflammatory agents to simultaneously release these bioactive molecules as they degrade. These novel bioengineered ear tubes will be a tunable device with improved biocompatibility (e.g., Cytocompatible, minimal or no inflammatory response), resorption rates, and advantageous drug eluting potential.

The disclosed ear tubes exhibit shape fidelity (e.g., Suitable geometry/comparable to commercial tubes) and mechanics comparable to commercial tympanostomy tubes while incorporating enhanced on-demand degradability and functionalization.

To eliminate the long-term consequences of extrusion-related complications, resorbable polymers are attractive options as fabrication materials. However, common synthetic polymers such as poly(lactic-co-glycolic acid) (PLGA) and poly(L-lactide) (PLL) degrade through a bulk hydrolytic process that results in the local accumulation of acidic byproducts and inflammation, which is non-ideal for the thin and delicate tympanic membrane. This disclosure concerns the use of natural biopolymers, such as silk fibroin protein and chitosan polysaccharide, that degrade into soluble amino acids/peptide fragments and oligosaccharides, respectively. Furthermore, because these biopolymers can be processed using aqueous-based manufacturing techniques, and have useful properties for drug stabilization and release, active pharmaceutical ingredients (APIs) may be incorporated into ear tubes prepared from these materials while maintaining their function and controlling their release rate over time.

Disclosed herein is an evaluation of tympanostomy tubes prepared from biopolymers as a suitable alternative to existing commercially available ear tubes, all of which are non-absorbable. Disclosed herein is the next-generation ear tube that demonstrates appropriate mechanical properties to be easily handled during operation, elutes antibiotics/steroids post-operation, maintains patency for the duration of implantation, and uniquely dissolves on demand.

Disclosed herein are ear tubes that can be manufactured using different techniques and possess appropriate mechanical properties. To improve scalability of the ear tube manufacturing process, while limiting waste material from subtractive manufacturing approaches like machining, a novel molding approach to manufacture small medical devices like ear tubes is disclosed. The silk or chitosan is deposited into a custom-made 3D printed mold where they are centrifuged to conform and fill the negative of an ear tube geometry. Subsequently, a polymerization bath is applied to the deposited material, resulting in a solid ear tube construct. As shown in FIG. 2, shape fidelity is maintained using this method. Disclosed herein are biopolymer formulations and mold designs, as well as postprocessing steps, that are optimized to obtain the targeted ear tube geometries and mechanical properties.

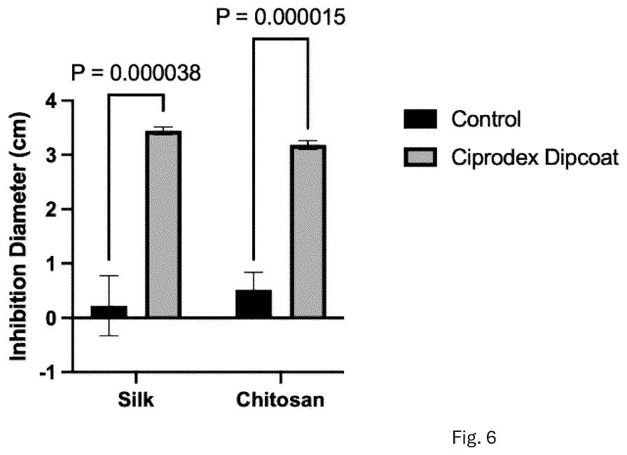

Drug loading and release (e.g., Maintenance of bioactivity, controllable rate/duration (3-7 days), and bioactivity are controllable based on formulation parameters. A significant advantage of natural polymers over their synthetic counterparts is the ability to load them with bioactive molecules like antibiotics or anti-inflammatory drugs. After ear tube implantation in patients, a regimen of Ciprodex is usually administered through the ear canal via otic topical drops. Ciprodex is a combination of an antibiotic, ciprofloxacin, to prevent infection; and a steroid, dexamethasone, to reduce inflammation. Adherence to otic medication regiment can be difficult, in particular when dealing with young children. As such, delivering these drugs locally through the ear tube would result in better outcomes for patients, in addition to allowing high drug concentration, increased efficacy, and limited side effects. Silk has been shown to stabilize and sequester bioactive molecules like antibiotics, enzymes, or growth factors, resulting in a more sustained action. FIG. 6 depicts that silk and chitosan discs could be manufactured and dipcoated with a silk solution containing Ciprodex. These discs were able to inhibit the growth of Staphylococcus aureus, as assessed by a disc diffusion test.

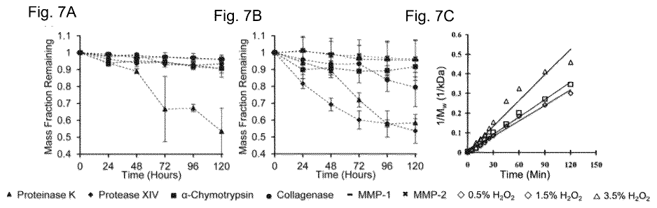

Degradation of biopolymers is a function of material properties. As mentioned above, a crucial way to limit complications relating to tympanostomy tubes is to allow a controllable and on demand degradation of the ear tube by the clinician. Previous work has shown that silk can be degraded in a tunable fashion through the action of proteolytic enzymes like protease XIV, as shown in FIGS. 7A and 7B. Likewise, chitosan has been shown to degrade in response to the action of hydrogen peroxide, at concentrations compatible with formulations commonly used in the ear canal (i.e., 3% or lower) as shown in FIG. 7C.

Taken together, the disclosure herein indicates that ear tubes can be manufactured from biopolymers like silk and chitosan, and that drug release and degradation can be controlled by various processing methods.

The disclosure herein improves upon an existing molding technique to consistently and accurately polymerize chitosan and silk materials into viable geometries with preferred mechanical properties. The mechanical properties of both materials can be tuned during polymerization through a variety of substrate and solvent compositions, as well as various additives. Material properties from the manufacturing process will also determine the degradation ability of silk and chitosan. Functionalization in the form of drug elution is disclosed through methods such as dip coating or incorporating drugs within the materials.

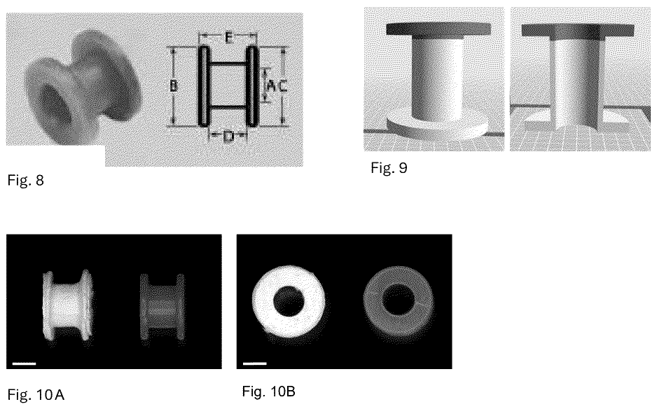

Disclosed herein are manufacturing methods for biomaterial-based ear tubes that mimic the size, geometry, and mechanical properties of existing commercial products. Example ear tubes approximately replicate the dimensions of “Collar Button” ventilation tubes (Summit Medical, see FIG. 8), which have an inner diameter of 1.3 mm, inner/outer flange diameters of 2.9 mm, inner flange distance of 1.4 mm, and total length of 2.1 mm. Using the centrifugal molding process described above, silk and chitosan solutions are polymerized into custom-made 3D printed molds of the desired ear tube geometries. The silk and chitosan are polymerized in high ionic strength salt bath (0.5 M dipotassium phosphate and 4 M sodium chloride) and strongly basic bath (5% w/v sodium hydroxide in 70% ethanol), respectively. The silk or chitosan is deposited into a custom-made 3D printed mold and centrifuged to conform to the mold. Subsequently, the polymerization bath is deposited into the mold and centrifuged again to polymerize the viscous silk/chitosan solution into the shape of an ear tube to eliminate the need for machining. Following complete polymerization (~24 hours), a controlled slow drying method will be used to retain shape fidelity of the ear tubes. Various molds may be used to achieve optimized ear tube geometry in coordination with material composition and drying processes. Quality control of ear tubes is performed by quantitative measurement of tube dimensions and qualitative imaging (e.g., scanning electron microscopy). To test the mechanical properties of materials, dog bones of silk and chitosan are prepared according to ASTM 638 standards. The mechanical properties of the constructs are evaluated by tensile testing of the bulk silk and chitosan materials on a universal testing machine (Instron 3366).

Disclosed herein are biomaterials incorporated into ear tubes that can degrade on-demand and exhibit degradation rates as a function of varied substrates and solvents. Disks were prepared (~6 mm diameter, ~1 mm height) using the same methods described above for ear tube manufacturing and evaluate degradation rates upon exposure to known agents (e.g., protease XIV, a non-specific mixture of serine proteases known to degrade silk through the cleavage of amide bonds, and hydrogen peroxide, a solution known to disrupt glycosidic linkages within chitosan structures). Different concentrations of protease XIV8 (1-6 U/mL) were examined and commercially available, commonly used concentration of hydrogen peroxide (3%). Test articles were immersed in these solutions and evaluated for changes in appearance and total mass loss over time, to obtain a complete degradation of the ear tube in 1-3 days, which is consistent with clinical needs for the management of retained ear tubes.

Disclosed herein are functionalized ear tubes with active ingredients (antibiotics, anti-inflammatories, clinically relevant drugs) to improve utility done by incorporating drugs through a dip coating process. Constructs were submerged within a 50/50 solution of 2% silk fibroin and Ciprodex (0.3% ciprofloxacin and 0.1% dexamethasone), a currently used combination ear drop medication to treat middle ear infections. Dip-coated constructs were allowed to dry to create a film before; the dip-coating process can be repeated up to 4 times to increase the amount of drug loaded. The dip coated constructs are water annealed to crystalize the silk to control the drug release profile, as previously described (See Hu, X. et al. Regulation of silk material structure by temperature-controlled water vapor annealing. Biomacromolecules 12, 1686-1696 (2011).). The water annealing process is optimized based on time and temperature, to obtain a sustained release of Ciprodex over 3-7 days, which should help with peri-operative complications.

In addition to or instead of the techniques described herein, different mold prototypes and designs, including different guided drying methodologies or subtractive manufacturing as well as altered concentrations and molecular weights of the silk and chitosan solutions may be used in creating the ear tubes. Furthermore, plasticizers can be added to create more flexible constructs and microparticles can be added to create stiffer constructs. Control over concentration and/or molecular weights can be used to address any issues with construct degradation. Disclosed herein is enhanced functionalization by increasing the frequency of the dip coating process and/or the concentration of ciprofloxacin and dexamethasone. Drugs can be incorporated directly within the constructs as well for a further extended release or higher concentration of release.

Disclosed herein is the biological response of ear tubes to determine impact of enhanced functionality in comparison to non-functionalized or commercial products. Assessments are carried out in models of increasing complexity, from in vitro cytocompatibility assays to subcutaneous dorsal implantation in a rat model to assess general biocompatibility, and finally implantation of ear tubes in a physiologically relevant animal model of the middle ear (chinchilla). Cell attachment and proliferation are used to demonstrate that test articles are not cytotoxic in vitro using standard bioassays before proceeding to animal studies. Following implantation, inflammatory responses are monitored using histological staining and monitoring of physiological health. For ear tube insertion, auditory function and healing over time is tracked.

Disclosed herein is cytocompatibility testing in vitro using standard bioassays. For in vitro assessment of cytocompatibility, disks of silk and chitosan following the processes described herein were completed. These test articles are seeded with L929 mouse fibroblasts, according to ISO 10993-5 standards. Cells are kept in culture for 24 hours after seeding, in MEM culture medium supplemented with 10% Fetal Bovine Serum (FBS), 4 mM glutamine, and 1% Antibiotic-Antimycotic. Cell adhesion is tracked by brightfield, confocal and scanning electron microscopy. General morphology, cell detachment, lysis and membrane integrity are visually assessed. Cell proliferation is quantified using Alamar blue. Cytocompatibility is assessed using the MTT protocol, through indirect contact, as well as direct contact. Negative control for cytotoxicity is fresh culture medium, and positive control is latex.

L929 mouse fibroblasts are able to adhere to the silk and chitosan constructs (e.g., Complete coverage of surface by fibroblasts after 24 hours), spread, as well as survive and proliferate over time (e.g., Increase in metabolism over 4 weeks; limited (<10%) dead cells after 3 days of culture), as seen by actin cytoskeleton staining (phalloidin), confocal microscopy, and SEM. In addition, a direct and indirect cytotoxicity assay was carried out (MTT bioassay), in accordance with ISO 10993-5 standard. A reduction in cell viability by no more than 30% was observed for cells in direct contact with constructs (24 hours) and after incubation in cell culture medium in contact with constructs for 24 hours. Cytotoxicity assays are also carried on the degradation byproducts that are formed during the degradation process, and in the functionalized materials developed herein, to assess potential cytotoxic effects of these byproducts and additives.

Disclosed herein is subcutaneous implantation of ear tubes in a rodent model to evaluate host response. The disks made of the silk or chitosan formulations disclosed herein were implanted subcutaneously dorsally in a rat model. Blank disks serve as controls, as do disks dip-coated with silk but without any drug loading. The final condition tested are constructs dip-coated with silk and Ciprodex. These materials are implanted subcutaneously to assess their general biocompatibility, long-term in vivo stability, cell/tissue infiltration, remodeling, and degradation. Briefly, an incision is made in the back of the rat. Sterilized silk and chitosan constructs are inserted, and the wound is closed using surgical clips. The implants and surrounding tissue are harvested and processed for histological analysis. The biocompatibility of the biomaterials is evaluated, i.e., the cell types interacting with the material, integration or degradation behavior, implant bed vascularization and immunological response. Study groups for rat dorsal subcutaneous implantation are: silk alone; silk dipcoated in silk, no drug; silk dipcoated in silk+Ciprodex; chitosan alone; chitosan dipcoated in silk, no drug; chitosan dipcoated in silk+Ciprodex. Six constructs are implanted in each rat. Six rats are implanted with the disks for all study groups and for each timepoint (see Table 1). Male and female animals were selected to account for sex differences.

TABLE 1

| Feature | Method | Result |
|---|---|---|
| Acute | Histological (H&E) | Signs of normal inflammation |
| inflammation | staining and imaging | 3 days after implantation; |
| | | no excessive or abnormal |
| | | inflammation |
| Chronic | Histological staining | No signs of inflammation |
| inflammation | (H&E) and imaging | ≥30 days after implantation |
| Physiological | Visual observation and | No signs of distress and |
| health | weight measurement | increased weight over time |
| In vivo | Histological staining | Ear tubes retain shape and |
| stability | and imaging | size for ≥30 days after |
| | | implantation |

Disclosed herein is an assessment for impact on hearing, local tympanic membrane tissue effects and in vivo degradability of ear tubes in a chinchilla model. The efficacy of bioresorbable silk and chitosan tympanostomy tubes in a chinchilla model was studied by assessing auditory function before and after tube placement and through periodic endoscopic examination of the implanted tubes, followed by histological processing of tympanic membrane (TM) tissues at study end. The chinchilla is chosen as the gold standard for these devices, due to similarities between the TM and ear canal in comparison to those of the human ear, as well as established methods of cochlear function testing. Hearing assessments are performed by recording the distortion product otoacoustic emissions (DPOAEs) and auditory brainstem response (ABR) before ear tube implantation and after resorption. Testing requires the placement of foam tipped earphones (a standard in audiological testing) into intact ear canals of the animals and the placement of three sterilized subdermal needle electrodes: one on the vertex, and one posterior and inferior to each pinna. The DPOAE is an automated test of peripheral auditory function, performed on a live but sedated animal, that assesses responses generated when the cochlea is stimulated simultaneously by two pure tone frequencies whose ratio in hertz is between 1.1 to 1.3. For ABR testing, tone pips of levels between 20 dB SPL (near threshold) and no larger than 80 dB SPL (loud conversational speech) are used to evoke a detectable waveform which can reliably establish auditory thresholds. Testing is performed binaurally. Animals are maintained on a surgical plane of anesthesia throughout by monitoring heart rate and toe-pinch withdrawal reflex. After auditory testing, sedated animals undergo M&T procedure bilaterally. The ability of the novel silk and chitosan tympanostomy tubes to maintain patency for a prolonged period is assessed, while signs of local inflammation in the eardrum are monitored endoscopically. Four weeks after the tubes are initially placed, and two weeks after resorption has been induced, each animal is anesthetized for a terminal experiment in which repeat DPOAE and ABR testing is performed, followed by harvesting of the tympanic membranes for histological analysis of its tissue structure to evaluate potential changes in response to the ear tube implantation and resorption. Six chinchillas were implanted for each timepoint. Male and female animals are selected to account for sex differences. Each animal serves as its own control for the ABR and DPOAEs, by carrying out a testing of auditory function before and after implantation. A commercially available ear tube is placed in the contralateral ear as an additional control.

TABLE 2

| Feature | Method | Result |
|---|---|---|
| Implantation and | Endoscopic imaging | Endoscopic visual confirmation |
| functional assessment | at each sedation | of successful implantation of the |
| | timepoint | ear tube in the tympanic |
| | | membrane; No signs of infection |
| | | or abnormal inflammation |
| | | surrounding the ear tube (Likert |
| | | scale); Tube remains in place |
| | | until resorption is induced (2 |
| | | weeks after implantation); |
| | | Tympanic membrane shows |
| | | signs of healing around the ear |
| | | tube 2 weeks after implantation |
| | | (Likert scale); Lumen of the ear |
| | | tube remains visible and patent |
| | | for the study duration |
| Auditory function | Auditory brainstem | No significant difference at the |
| | response (ABR); | various timepoints (pre- |
| | Distortion Product | implantation, post-implantation, |
| | Otoacoustic Emissions | post-resorption) or between ears |
| | (DPOAEs) | at each timepoint in comparison |
| | | to a commercially available ear |
| | | tube implanted in the |
| | | contralateral ear as a control |
| Tympanic | Histological | Moderate to high levels of |
| membrane healing | analysis (H&E) | healing 2 weeks after resorption |
| | | of the ear tube (healed/non- |
| | | healed determined |
| | | endoscopically) |

Ear tubes demonstrated acceptable biocompatibility, including lack of inflammation and suitable healing as assessed via endoscopic exam and use of a Likert scale. Further, hearing thresholds were unaffected by the implantation or resorption of the ear tubes. Additional in vitro testing cell types include human middle ear epithelial cells (MEEpiC, ScienCell) in addition to or instead of L929 fibroblasts. Control over cell response can be modified by addition and/or increased concentration of bioactive agents (e.g., steroids).

In some embodiments, silk is more stable over time, and less prone to rapid degradation on command. To take advantage of the properties of both biopolymers, composite ear tubes can be made by combining both materials, as shown in FIG. 9. For controlled release of the ear tube for example, the inner flange (on the middle ear side) could be manufactured out of chitosan, allowing rapid degradation on command, while the tube and outer flange (on the ear canal side) could be manufactured out of silk, which could be degraded enzymatically over a longer timeframe. This approach would ensure that the ear tube is released into the ear canal rather than the middle ear, making it easily removable by the patient/doctor.

### Example 2

Disclosed herein is a fully biopolymer ear tube that remained intact and did not migrate into the middle ear, degraded on demand, and remained patent supporting safety, performance, and novel function.

The current medical landscape sees millions of procedures annually that rely on synthetic, non-resorbable implants, or implants that have inflammatory byproducts leading to complications or removal surgeries. Implants made of natural biopolymers offer promising alternatives but are widely underutilized because they are limited by current manufacturing techniques, which are often harsh, expensive, or require additives, effectively compromising the performance features that natural biopolymers offer in the first place. Unlocking the potential of natural biopolymers can improve clinical outcomes for millions of patients undergoing various surgical procedures or receiving implants. This is especially true for tympanostomy tubes, a widely utilized implant exceeding 1 million placements annually in the US, and which exemplifies the pressing need for enhanced features. Currently, for children that have chronic ear infections, surgeons insert non-degradable fluoroplastic or silicone tubes as a standard of care. The sheer number of tubes and performance inconsistencies results in a significant number of patients having unpredictable complications (more than 50,0000 repeat surgeries for removal or replacement and up to 38% of children have reported eardrum scarring). Tubes may also become clogged or coated with biofilm, resulting in non-ventilation of the middle ear or persistent otorrhea (ear drainage), in which case parents must administer antibiotics. Given the volume of procedures, complications from tubes represent a significant burden on health. Furthermore, race/ethnicity and socioeconomic status impact tube placement such that white children and children with uninterrupted insurance coverage are more likely to undergo tube placement. Despite outcomes, limited innovation has been seen in this implant category since the 1950s.

Disclosed herein is the first fully biopolymeric ear tube. This sophisticated 3D shape, made from pure natural chitosan using a novel manufacturing process is nearly indistinguishable from commercially available ear tubes (FIG. 10A). Disclosed herein is the successful use of chitosan to produce ear tubes with appropriate geometry, shape fidelity, and that are visually almost indistinguishable from commercially available ear tubes. However, the biopolymer ear tubes offer unique benefits for ear surgeries, including: 1) On-Demand Degradation: Can perform with all the features of conventional tubes but also provide the option to be removed non-surgically, giving surgeons full control and sparing children the risk of repeat surgery under general anesthesia; 2) Antimicrobial Properties: Naturally antimicrobial and can elute antibiotics/steroids; and 3) Enhanced Patient Care: Aims to reduce complications, follow-up care burdens, and improve healing outcomes. Beyond ear tubes, other implantable medical devices are anticipated to be enabled by the disclosure herein, benefiting patients and the healthcare system at large.

The disclosure herein concerns certain results that: 1) Confirm biocompatibility of implanted ear tube (via Otoscope assessment and Histological evaluation); 2) Confirm ear tube patency (via Tympanometry and Otoscope assessment); 3) Characterize kinetics of on-demand degradation (via Otoscope assessment); 4) Rule out potential cytotoxicity (via Otoscope assessment, Histological evaluation, and DPOAEs); 5) Confirm tympanic membrane healing after degradation (via Otoscope assessment and Histological evaluation); and 6) Assess long-term capabilities of ear tubes (via Otoscope assessment and Histological evaluation).

The chinchilla animal model is well-established in the field of hearing science due to their behavioral, anatomical, and physiological characteristics. These include similarities with human hearing frequency and intensity sensitivity, the ability to be trained behaviorally with acoustic stimuli relevant to human hearing, their docile nature that allows many physiological measures to be made in an awake state, physiological robustness that allows for data to be collected from all levels of the auditory system, and the ability to model various types of conductive and sensorineural hearing losses that mimic pathologies observed in humans. Twelve chinchillas were selected for this study and were divided into three groups: a training group, a short-term group, and a long-term group. Each animal received two ear tubes, one in each ear. Prior to implantation, hearing was assessed using the following methods: 1)

Tympanometry: This was performed to measure the integrity of the tympanic membrane; and 2) Distortion Product Otoacoustic Emissions (DPOAEs): This test was used to assess the health of the outer hair cells, providing a baseline for hearing function and ear health.

Animal Surgery, Treatment Groups, and Imaging; Training Surgery (n=3)

This group was established to practice auditory testing procedures and to train the team on hearing measurements and surgeon on the implantation technique for the ear tubes. No data collection was planned to be performed on this group. The focus was solely on perfecting the surgical technique and ensuring consistent auditory testing methodology on the chinchillas.

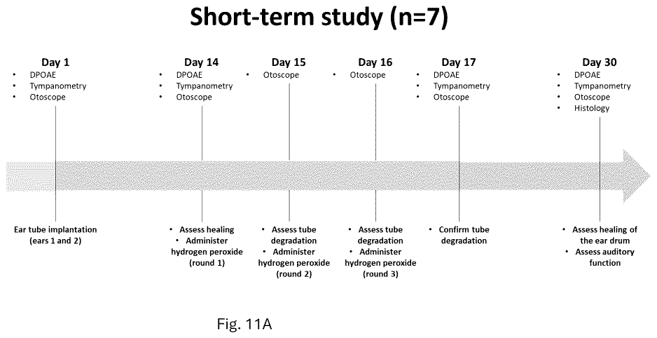

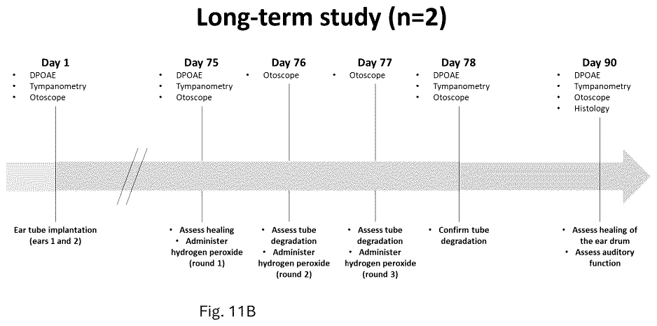

Short-Term Group (n=7); See FIG. 11A

Implantation: six animals (12 ears) were planned to be implanted with ear tubes.

Treatment Regimen: Two weeks after implantation, a 3% hydrogen peroxide solution would be administered in both ears (3 mL deposited in the ear canal, dwell time: 10 min). The administration schedule was as follows: 3 consecutive days of treatment, 4 days without treatment, followed by another 3 consecutive days of treatment.

Evaluation: Otoscopic examinations would be carried out throughout this process to evaluate ear tube degradation.

Long-Term Group (n=2); See FIG. 11B

Implantation: four animals (4 ears) implanted with ear tubes.

Initial Evaluation: Three weeks after implantation, otoscopic examinations performed to rule out any potential ototoxicity of the ear tubes.

Degradation Test: Eight weeks after implantation, degradation induced using 5% hydrogen peroxide.

Observation Period: Animals observed up to a 12-week timepoint.

Evaluation: Otoscopic examinations conducted throughout to evaluate tube degradation.

### Hearing Assessments & Histology

### Short-Term Group:

Post-Treatment Hearing Assessment: Four weeks after implantation (two weeks after degradation was initiated), hearing assessed and compared to baseline measurements.

Histological Assessment: After hearing assessment, the animals euthanized. Their heads collected for processing and histological assessment of the tympanic membrane.

### Long-Term Group:

Intermediate Hearing Assessment: Eight weeks after implantation, ear tubes examined using an otoscope, and hearing assessed.

Degradation and Final Assessment: Degradation induced using 5% hydrogen peroxide. Twelve weeks after implantation (four weeks after degradation), hearing reassessed. Animals then euthanized, and heads collected for processing and histological assessment of the tympanic membrane.

This methodology ensured thorough training of the technical and surgical team, rigorous testing of the ear tubes, and comprehensive evaluation of their safety, tolerability, and degradation kinetics.

### Surgeon Feedback

Although routinely carried out by many ENT surgeons, ear tube implantation is a delicate procedure, requiring precise maneuvering in the ear canal and placement in the thin and fragile tympanic membrane. It is critical that the handling of the ear tubes is suitable for ENT surgeons. This is particularly important, as most mechanical forces will be applied by the ear tube during handling and implantation. Once implanted, little mechanical force is applied to the ear tube by the tympanic membrane.

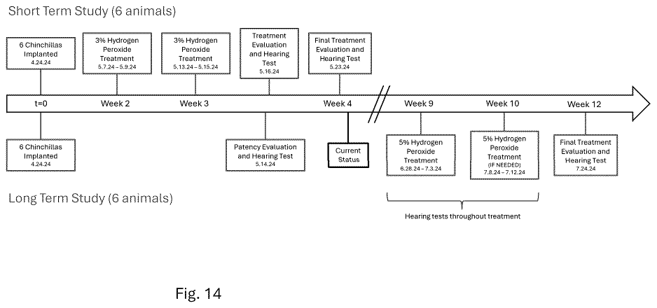

The surgeon implanting the ear tubes found them to be indistinguishable from traditional tubes, and successfully carried out the implantation of all 24 ear tubes of the chinchilla study, without any issue or need for adjustment. Given the success of the surgeries, the Training Animals were incorporated into the study, allowing the collection of data from 6 animals (12 ears) for each/both the short-term and long-term study groups (FIG. 14).

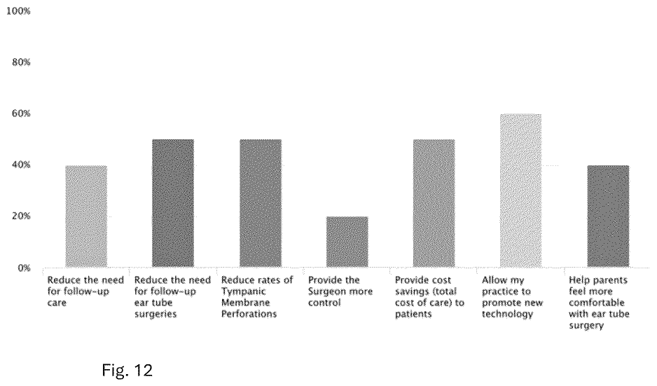

The desire for degrade-on-demand ear tubes was characterized using an anonymous survey to 10 ENT surgeons who commonly use ear tubes using InCrowd research platform. In response, surgeons replied they would Frequently or Always (60%) insert degrade-on-demand Ear Tubes over non-degradable grommet tubes. Others replied they would Occasionally (40%) insert degrade on-demand tubes, while no surgeon answered Rarely or Never. Furthermore, 70% of surgeons indicated they would pay more for such tubes, compared to non-degradable options. Benefits recognized by the Surgeons included: Allowed my practice to promote new technology; Reduced the need for follow-up care; Help parents feel more comfortable with ear tube surgery (FIG. 12).

### Degrade On-Demand Ear Tubes are Biocompatible

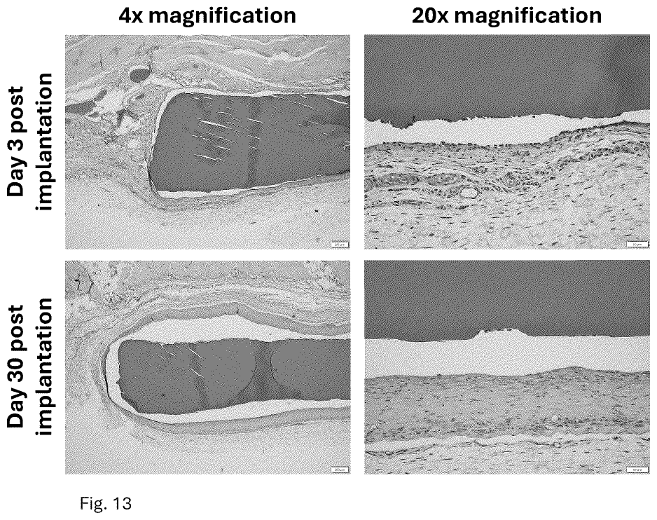

Biocompatibility was evaluated in vitro through cytotoxicity testing, and in vivo through subcutaneous implantation of test materials in a rat model. Chitosan materials were implanted subcutaneously in a rat model to assess their general biocompatibility (acute and chronic inflammation) by histological examination of hematoxylin- and eosin-stained slides (FIG. 13). Three days after implantation, the chitosan implants are intact in shape, and the margins of the implants are lined by mostly a single layer of small macrophages. The surrounding loose connective tissue contains a low to moderate density infiltrate of macrophages and fewer to minimal neutrophils. Inflammation is minimal to mild around the implants. At day 30 post implantation, the implants are still intact with minimal scalloping of edges. There is only mild fibroplasia in the connective tissue immediately surrounding the implants and only small numbers of macrophages are present around the implants. These results confirm that the materials are biocompatible and do not elicit an adverse inflammatory reaction (acute or chronic) after implantation.

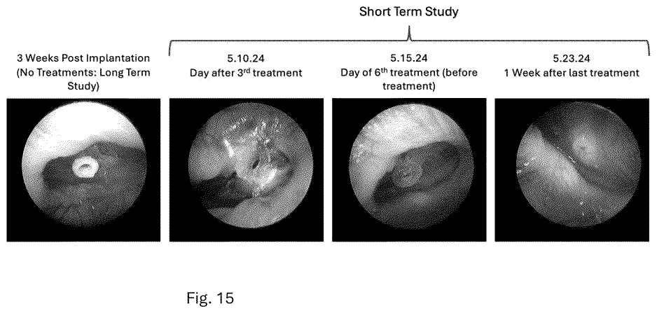

To build on this work, ear tube biocompatibility was evaluated in the chinchilla preclinical model through implantation in the tympanic membrane and confirmed through (1) otoscopic examination at different timepoints after implantation and (2) tissue collection and histological analysis after euthanasia (FIG. 15).

It was surprisingly discovered during this work that processing the explanted tympanic membrane for histology is a unique process compared to conventional soft tissue harvesting as it requires removing the mineralized tissue from the surrounding bone (a lengthy process). While traditional harvest for H&E could have been done, we chose to elevate our technique to this because it enables obtaining high-quality pictures of the tympanic membrane.

Degrade On-Demand Ear Tubes Maintain their Integrity and Patency In Vivo

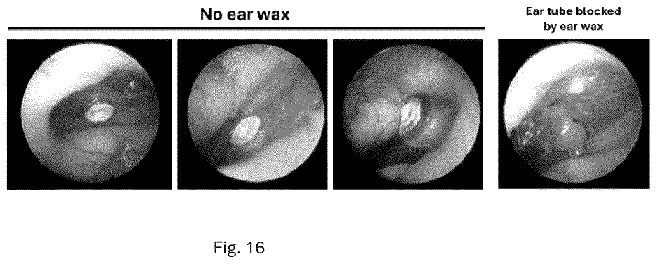

The purpose of the ear tube is to equalize pressures between the ear canal and the middle ear, as well as allow drainage of fluid out of the middle ear, to prevent infection. Ear tube blockage by mucin, blood, granulation, or wax can occur after implantation. This is not uncommon but can cause the tubes to become ineffective. Monitoring ear tubes after implantation enables checking for patency and ear tube integrity. In the study, one animal seemed to present increased ear wax production, blocking the ear tube, as shown in FIG. 16, right. This represented less than 10% of ears in this study and both cases came from the same animal.

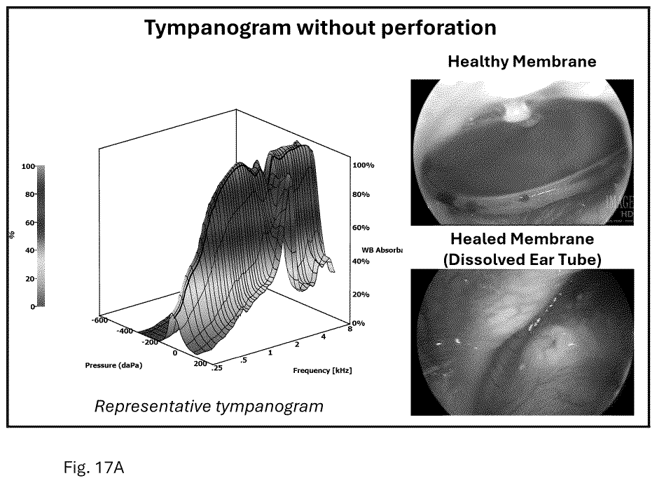

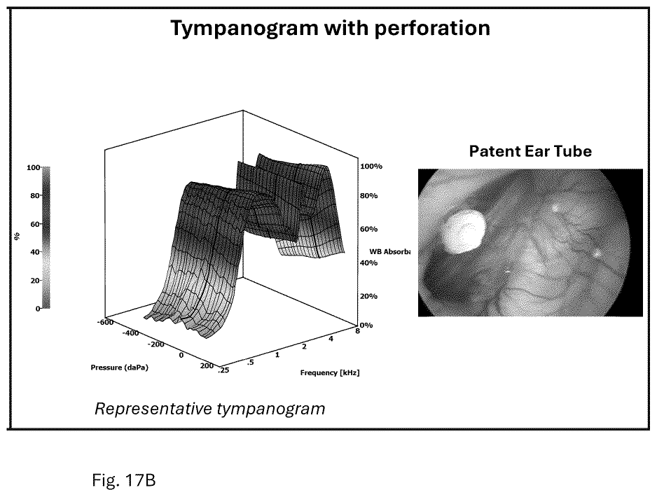

Using the long-term group, three weeks after implantation, otoscope observations (FIG. 16) confirmed that all ear tubes in this group were still in place and did not appear altered. Otoscope observations and tympanometry measurements indicate that our ear tubes can remain patent after implantation. A representative set of otoscope pictures and tympanometry data is shown in FIG. 17A and FIG. 17B.

### Degrade On-Demand Ear Tubes Reliably Degrade On-Demand In Vivo

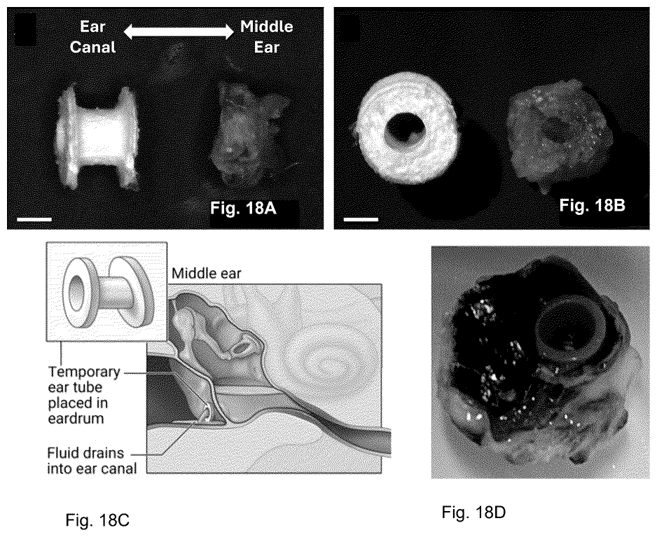

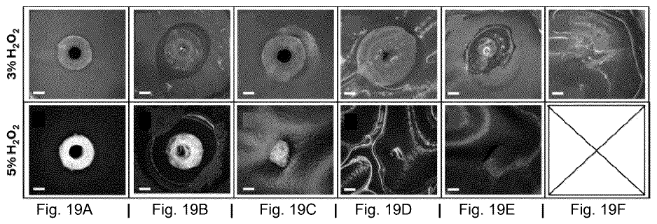

In vitro characterization of ear tube degradation had established that the ear tubes degrade with three daily 10-minute treatments with 3% hydrogen peroxide (FIG. 19, top). One of the goals of this study was to better understand in vivo degradation kinetics. In the short-term group, it was observed that 7 out of 12 tubes degraded after treatments with 3% hydrogen peroxide (confirmed through otoscopic evaluation and tympanometry). While this is considered rare, one of the extruded tubes was collected from the chinchilla ear canal (FIG. 18D). Interestingly, the tube appears to have absorbed ear wax, which has recognized antimicrobial properties. Furthermore, one side of the tube flanges is more degraded than the other side, which is believed to be the inner flange (middle ear facing) side because it could not have extruded with the other side's geometry intact as it was observed. This may indicate that the ear canal (outer flange side) is protected slightly from degradation-perhaps by earwax- and may have a positive effect for preventing any tube entry into the middle ear space. Five of 12 tubes experienced partial degradation and may have extruded after an additional hydrogen peroxide treatment. These results confirm the feasibility of in vivo degradation of the ear tubes.

Treatment may be optimized for faster degradation rates, to improve patient compliance. At the 8 week timepoint, the administration regiment can be adjusted to accelerate degradation. Specifically, by switching from 3% to 5% hydrogen peroxide 5% hydrogen peroxide is still considered safe for the eardrum and does not represent a significant risk for the animals. In vitro, all tubes can be degraded in vitro by the middle/end of the second daily treatment, as shown in FIG. 19. The in vitro results translate to accelerated degradation in vivo.

### Degrade On-Demand Ear Tube Implantation and Degradation do not Cause Loss of DPOAE

DPOAEs are indirect measures of outer hair cell integrity and function. Otoscope images and DPOAE measurements were taken at different stages: (1) before implantation (baseline), (2) three weeks after implantation (before degradation), and (3) 1 week after degradation.

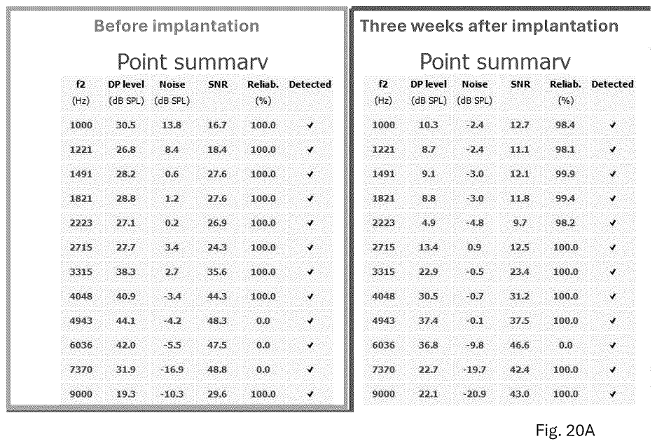

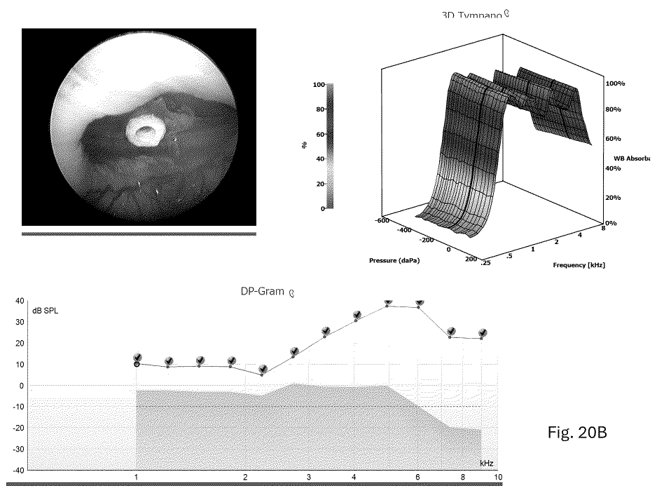

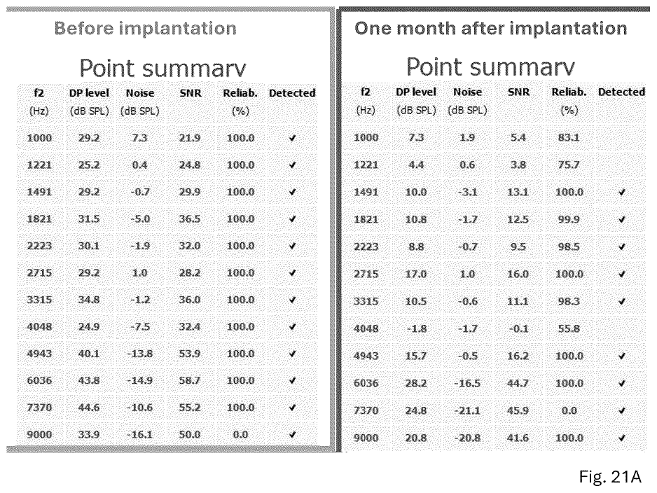

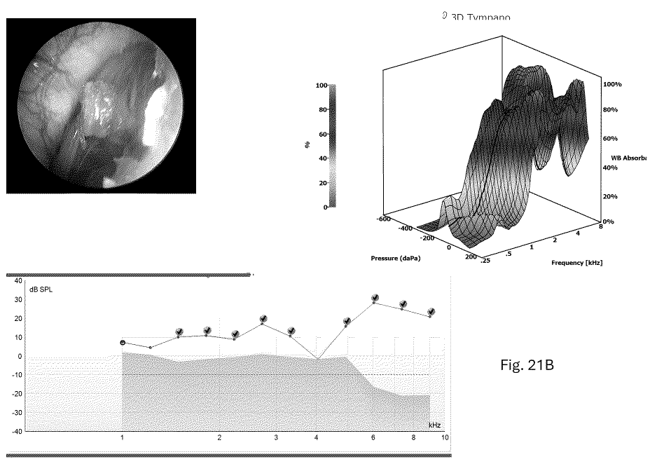

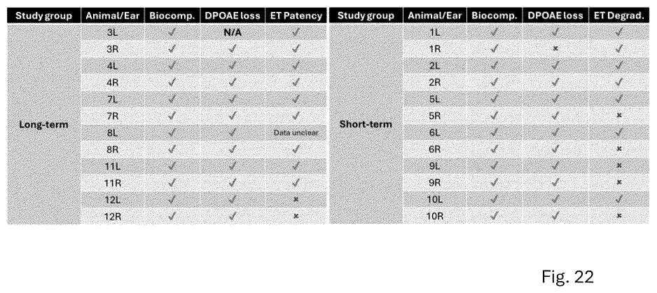

Representative curves and tables of the DPOAE measurements are presented on FIG. 20A, the bottom panel of FIG. 20B, FIG. 21A, and the bottom panel of FIG. 21B.

In the study, 100% of ears (n=12) showed no change or low-frequency change in DPOE amplitude after implantation of Chitosan ear tubes, which is consistent with anticipated DPOAE changes after conventional ear tube implantation. 92% of all degraded tubes (11/12) showed no change or low-frequency change in DPOAE from baseline indicating intact outer cell integrity and function.

Only one ear showed a loss in DPOAEs across a broad frequency range after degradation and tube extrusion. It is possible that this ear could have benefited from longer healing times (>1 week) after implantation to fully assess DPOAE and hearing function. The general hearing loss tendencies of chinchillas are also unknown, and larger samples sizes may be needed to account for natural hearing loss for device performance assessments. For example, one animal had a very low amplitude DPOAE signal at baseline in one ear, potentially indicating hearing dysfunction prior to study entry.

Overall, the data suggests that the novel ear tubes have (1) acute performance that is similar to traditional ear tubes and (2) the degradation process (hydrogen peroxide) does not introduce observable damage to outer hair cell integrity, hearing loss, or cochlear dysfunction. Furthermore, this data suggests that the process of tube degradation and byproducts do not cause ototoxicity.

Evidence of Low Perforation Rate (i.e. Tympanic Membrane Healing) Measured by Tympanogram

Otoscope observations and tympanometry measurements after degradation suggest that the tympanic membrane is able to heal after ear tube degradation (FIG. 15). Direct observation with the otoscope is difficult, but achievable. This difficulty is due to the convoluted nature of the chinchilla ear canal.

Long-term capabilities: The ear tubes of the long-term group are still implanted successfully, with an upcoming euthanasia timepoint 12 weeks after implantation. No animals have been sick or lost. These results enable confirmation of (1) ear tube integrity after several (10) weeks of implantation, (2) on-demand degradation of ear tubes (administrations of 5% hydrogen peroxide), (3) tympanic membrane healing two weeks after degradation, and (4) hearing function.

### Additional Data

a. Cytotoxicity

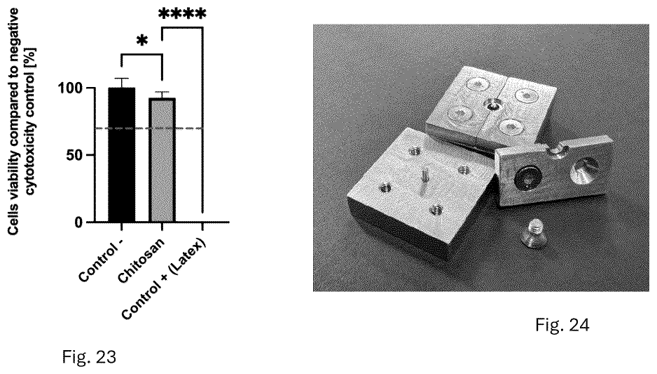

In vitro, L929 murine fibroblasts were seeded on discs of chitosan, and cell metabolism was assessed after 6 days of culture using the PrestoBlue assay. Metabolism was used as a proxy for cell viability, with the appropriate negative and positive controls, as outlined in 10993-5:2009—Biological evaluation of medical devices. A viability reduction greater than 30% (i.e., viability reduced to <70% of the blank) was considered as evidence of cytotoxicity. As shown in FIG. 20B, the biomaterials were not cytotoxic, as evidenced by the cell viability of 92% (FIG. 23).

b. Refining the Manufacturing Process

Much of the workflow involves using 3D printed molds that are manufactured as needed. This allows a dynamic approach when it comes to prototyping, adjusting designs, or making custom parts (e.g., for personalized care). Nevertheless, machined stainless steel molds present advantages for the mass manufacturing of parts, due to the low tolerances of machining, the stability of steel for repeated use, and the ease of sterilization. FIG. 24 depicts stainless steel molds that are suitable for the repeated manufacturing of ear tubes and may be useful in scaling up manufacturing.

c. Mechanical Properties

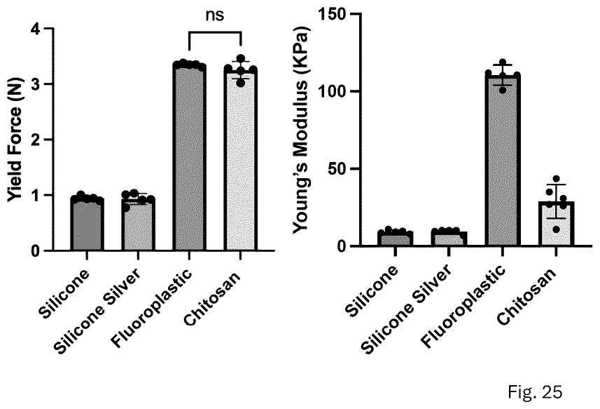

Uniaxial compression testing of the ear tubes was carried out, as well as three commercially available Medtronic products (Sheehy-Type Collar Button Ventilation Tube, Microgel (ref: 1083302), Sheehy-Type Collar Button, Activent Silicone (ref: 1026145), and Donaldson Fluoroplastic Ventilation Tube (ref: 1015101)). As shown in FIG. 25, the disclosed ear tubes have a yield force that is on par with fluoroplastic tubes, and a Young's modulus that is between silicone and fluoroplastic tubes. This, combined with the surgeon feedback regarding their handling capabilities, indicates that the disclosed ear tubes are suitable for implantation.

d. Functionalization Potential
a. Drug Loading and Release

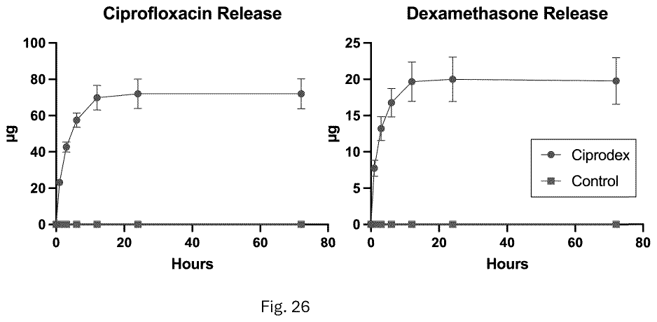

To assess the ability to functionalize the ear tubes, they were soaked in Ciprodex (0.3% ciproflocaxin and 0.1% dexamethasone), which is commonly administered to patients in the form of otic drops after ear tube implantation. As shown in FIG. 26, both compounds were released by the chitosan in approximately 24 hours, suggesting that ear tubes can be loaded with Ciprodex for the prophylactic treatment of post-tympanostomy tube otorrhea. Chitosan's cationic nature and the pH of Ciprodex may be leveraged to improve loading, or the Ciprodex may be incorporated in the manufacturing process for a more sustained release.

b. Kirby Bauer Disc Diffusion Assay

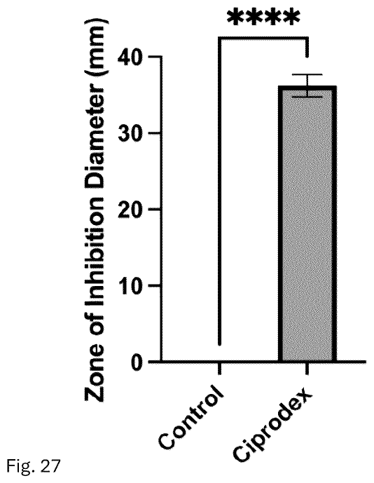

It was confirmed that the loaded Ciprofloxacin maintained its bioactivity by characterizing its ability to inhibit the growth of Xen29, a Staphylococcus aureus strain. As shown in FIG. 27, the Ciprodex had a strong inhibitory effect on S. aureus growth.

To estimate the concentration of ciprofloxacin released by the chitosan, a standard curve was established using blank paper discs and known dilutions of Ciprodex. The measured concentration is ~1700 μg/mL, which largely exceeds the MIC values of relevant middle ear-infecting organisms. This suggests that the ear tubes can be effectively functionalized with Ciprodex for the prophylactic treatment of acute otitis following ear tube implantation.

While the disclosure has been disclosed in connection with the preferred embodiments shown and described in detail, various modifications and improvements thereon will become readily apparent to those skilled in the art. Accordingly, the spirit and scope of the present disclosure is not to be limited by the foregoing examples, but is to be understood in the broadest sense allowable by law.

In addition to the features described above and elsewhere herein, the present disclosure also includes the following clauses:
1. An ear tube comprising at least one biopolymer, the ear tube having at least one characteristic centrifugal property selected from the group consisting of:
   i) a uniform density across at least a portion of the ear tube;
   ii) at least a portion of the ear tube is optically nontransparent;
   iii) at least a portion of the ear tube is free of exterior layering;
   iv) at least a portion of the ear tube is free of interior layering;
   v) at least a portion of the ear tube is interior-void-free;
   vi) at least a portion of the ear tube is free of cylindrical extrusion gaps; and
   vii) at least a portion of the ear tube is free of layering.
2. The ear tube of clause 1, wherein at least one of an outer flange or an inner flange of the ear tube is angled.
3. The ear tube of clause 2, wherein the at least one of the outer flange or inner flange defines a plane which is inclined at an angle with respect to a plane disposed perpendicular to an axis of a main tube of the ear tube.
4. The ear tube of clause 3, wherein the angle is between 15 degrees and 45 degrees, including but not limited to, at least 15 degrees, at least 30 degrees, or at least 45 degrees.
5. The ear tube of clause 2, wherein at least one of the outer flange or inner flange comprises an insertion protuberance.
6. The ear tube of clause 1, wherein an outer flange and an inner flange of the ear tube are at least one of same or different with respect to at least one of a thickness, a relative angle, a shape, an orientation, or a geometry.
7. The ear tube of clause 1, wherein the ear tube has a uniform diameter throughout its length.
8. The ear tube of clause 1, wherein the ear tube has a non-uniform diameter along its length.
9. The ear tube of clause 1, wherein the ear tube tapers along its length.
10. The ear tube of clause 1, wherein the ear tube has a uniform density throughout.
11. The ear tube of clause 1, wherein the ear tube comprises a cross-sectional profile that is uniform throughout the ear tube.
12. The ear tube of clause 11, wherein the uniform cross-sectional profile is determined by at least one of a visual inspection, scanning electron microscopy, fluorescence microscopy, DSC, TEM, XRD, EPMA, or FTIR.
13. The ear tube of clause 11, wherein the cross-sectional profile relates to at least one of a density, an absence of voids, a consistent pattern, or a chemical profile.
14. The ear tube of clause 1, wherein a main tube of the ear tube has an axial length at least several times its diameter.
15. The ear tube of clause 1, wherein a main tube of the ear tube has an inner diameter of between 0.1 and 2.9 mm, between 0.1 and 2.5 mm, between 0.1 and 2 mm, between 0.1 and 1.5 mm, between 0.1 and 1 mm, between 1.0 and 3 mm, between 1.0 and 2 mm, or between 1.0 and 1.5 mm, including but not limited to, at least 0.1 mm, at least 0.5 mm, at least 0.6 mm, at least 0.7 mm, at least 0.8 mm, at least 0.9 mm, at least 1.0 mm, at least 1.1 mm, at least 1.2 mm, at least 1.3 mm, at least 1.4 mm, at least 1.5 mm, at least 2.0 mm, at least 2.5 mm, at least 3.0 mm, or at least 3.8 mm.
16. The ear tube of clause 1, wherein a main tube of the ear tube has an outer diameter of between 0.1 and 2.9 mm, between 0.1 and 2.5 mm, between 0.1 and 2 mm, between 0.1 and 1.5 mm, between 0.1 and 1 mm, between 1.0 and 3 mm, between 1.0 and 2 mm, or between 1.0 and 1.5 mm, including but not limited to, at least 0.1 mm, at least 0.5 mm, at least 0.6 mm, at least 0.7 mm, at least 0.8 mm, at least 0.9 mm, at least 1.0 mm, at least 1.1 mm, at least 1.2 mm, at least 1.3 mm, at least 1.4 mm, at least 1.5 mm, at least 2.0 mm, at least 2.5 mm, at least 3.0 mm, or at least 3.8 mm.
17. The ear tube of clause 1, wherein the ear tube has an inner flange, the inner flange diameter is between 1.0 mm and 10.0 mm, between 2.0 mm and 9.0 mm, between 2.0 mm and 7.0 mm, or between 3.0 mm and 6.0 mm, including but not limited to, at least 1 mm, at least 1.5 mm, at least 2.0 mm, at least 3.0 mm, at least 4.0 mm, at least 5.0 mm, at least 6.0 mm, at least 7.0 mm, at least 8.0 mm, at least 9.0 mm, or at least 10.0 mm.
18. The ear tube of clause 1, wherein the ear tube has an outer flange, the outer flange diameter is between 1.0 mm and 10.0 mm, between 2.0 mm and 9.0 mm, between 2.0 mm and 7.0 mm, or between 3.0 mm and 6.0 mm, including but not limited to, at least 1 mm, at least 1.5 mm, at least 2.0 mm, at least 3.0 mm, at least 4.0 mm, at least 5.0 mm, at least 6.0 mm, at least 7.0 mm, at least 8.0 mm, at least 9.0 mm, or at least 10.0 mm.
19. The ear tube of clause 1, wherein a wall of a main tube of the ear tube has a thickness of between 0.1 mm and 0.5 mm, between 0.1 mm and 0.4 mm, or between 0.3 mm and 0.5 mm, including but not limited to, at least 0.1 mm, at least 0.2 mm, at least 0.3 mm, at least 0.4 mm, or at least 0.5 mm.
20. The ear tube of clause 1, wherein a distance between a top surface of an inner flange of the ear tube and a bottom surface of an outer flange of the ear tube is between 0.05 mm and 12.0 mm, between 0.1 mm and 7.5 mm, between 1 mm and 7.5 mm, between 2.0 mm and 5.0 mm, including but not limited to, at least 0.05 mm, at least 0.1 mm, at least 0.5 mm, at least 1.0 mm, at least 2.0 mm. at least 3.0 mm, at least 4.0 mm, at least 5.0 mm, at least 7.5 mm, at least 10.0 mm, or at least 12.0 mm.
21. The ear tube of clause 1, wherein a total length of the ear tube is between 0.5 and 3 mm, between 0.5 and 2.5 mm, between 1 and 2.5 mm, between 1 and 2 mm, between 1 and 1.5 mm, between 2.0 mm and 5.0 mm, or between 3.0 mm and 8.0 mm, including but not limited to, at least 1.5 mm, at least 2.0 mm. at least 3.0 mm, at least 4.0 mm, at least 5.0 mm, at least 7.5 mm, or at least 8.0 mm.
22. The ear tube of clause 1, wherein an outer flange and an inner flange of the ear tube are formed from a same biopolymer.
23. The ear tube of clause 1, wherein an outer flange and an inner flange of the ear tube are formed from different biopolymers.
24. The ear tube of clause 1, wherein an outer flange and an inner flange of the ear tube are formed from at least two biopolymers, each of the at least two biopolymers defining a thickness of the flange.
25. The ear tube of clause 1, wherein a main tube of the ear tube is formed from a single biopolymer.
26. The ear tube of clause 1, wherein a main tube of the ear tube is formed from a plurality of biopolymers, wherein a first biopolymer of the plurality of biopolymers forms an inner surface of the main tube and a second biopolymer of the plurality of biopolymers is disposed circumferentially around the first biopolymer and forms an outer surface of the main tube.
27. The ear tube of clause 1, wherein the ear tube comprises a first biopolymer forming at least a portion of a main tube of the ear tube and at least a portion of at least one flange, and a second biopolymer forming at least a portion of the main tube of the ear tube and at least a portion of at least one flange.
28. The ear tube of clause 1, wherein an inner flange of the ear tube comprises chitosan and a main tube of the ear tube and outer flange comprise silk.
29. The ear tube of clause 1, wherein the ear tube comprises at least one biopolymer, the at least one biopolymer is silk fibroin.
30. The ear tube of clause 1, wherein the ear tube comprises at least one biopolymer, the at least one biopolymer is at least one of silk fibroin or chitosan polysaccharide.
31. The ear tube of clause 1, wherein the ear tube comprises silk fibroin and chitosan polysaccharide.
32. The ear tube of clause 1, wherein the ear tube comprises silk fibroin and alginate.
33. The ear tube of clause 1, wherein the ear tube comprises at least one biopolymer, the at least one biopolymer is an aqueous silk solution, an aqueous alginate solution, an aqueous fibrinogen solution, an aqueous chitosan solution, or an aqueous collagen solution.
34. The ear tube of clause 1, wherein the ear tube is formed via centrifugal molding.
35. The ear tube of clause 1, wherein the ear tube is formed via thermal molding and machining.
36. The ear tube of clause 1, wherein the ear tube is formed via centrifugal molding, thermal molding, and machining.
37. The ear tube of clause 1, wherein the ear tube comprises at least one biopolymer, the at least one biopolymer is assembled via an aqueous-based polymerization.
38. The ear tube of clause 1, wherein the ear tube comprises at least one additive.
39. The ear tube of clause 38, wherein the at least one additive is at least one of a bioactive molecule, a hydrophilic plasticizer, a hydrophobic plasticizer, a pigment, or a dye.
40. The ear tube of clause 39, wherein the bioactive molecule is an active pharmaceutical ingredient, an antibiotic, an anti-inflammatory agent, a steroid, a growth factor, or an enzyme.
41. The ear tube of clause 38, wherein the at least one additive is dip-coated onto the ear tube.
42. The ear tube of clause 41, wherein the dip-coated ear tube is subjected to water annealing.
43. The ear tube of clause 38, wherein the additive is dissolved in a biopolymer solution before dip-coating.
44. The ear tube of clause 38, wherein the ear tube is impregnated with the at least one additive.
45. The ear tube of clause 40, wherein the enzyme is at least one of a proteolytic enzyme, a serine protease, protease XIV, proteinase K. alpha-chymotrypsin, collagenase, MMP-1, MMP-2, or glucose oxidase.
46. The ear tube of clause 1, wherein the ear tube is designed to degrade on demand.
47. The ear tube of clause 46, wherein degradation commences when the ear tube is exposed to a solvent.
48. The ear tube of clause 47 wherein the solvent is at least one of a glucose solution, hydrogen peroxide, carbamide peroxide, an alcohol, a calcium chelator, or EDTA.
49. The ear tube of clause 1, wherein the ear tube is a tympanostomy tube.
50. An ear tube comprising at least one material assembled via an aqueous solution, the ear tube having at least one characteristic centrifugal property selected from the group consisting of:
   i) a uniform density across at least a portion of the ear tube;
   ii) at least a portion of the ear tube is optically nontransparent;
   iii) at least a portion of the ear tube is free of exterior layering;
   iv) at least a portion of the ear tube is free of interior layering;
   v) at least a portion of the ear tube is interior-void-free;
   vi) at least a portion of the ear tube is free of cylindrical extrusion gaps; and
   vii) at least a portion of the ear tube is free of layering.
51. The ear tube of clause 50, wherein at least one of an outer flange or an inner flange is angled.
52. The ear tube of clause 51, wherein the at least one of the outer flange or inner flange defines a plane which is inclined at an angle with respect to a plane disposed perpendicular to an axis of a main tube.
53. The ear tube of clause 52, wherein the angle is between 15 degrees and 45 degrees, including but not limited to, at least 15 degrees, at least 30 degrees, or at least 45 degrees.
54. The ear tube of clause 51, wherein at least one of the outer flange or inner flange comprises an insertion protuberance.
55. The ear tube of clause 50, wherein an outer flange and an inner flange of the ear tube are at least one of same or different with respect to at least one of a thickness, a relative angle, a shape, an orientation, or a geometry.
56. The ear tube of clause 50, wherein the ear tube has a uniform diameter throughout its length.
57. The ear tube of clause 50, wherein the ear tube has a non-uniform diameter along its length.
58. The ear tube of clause 50, wherein the ear tube tapers along its length.
59. The ear tube of clause 50, wherein the ear tube has a uniform density throughout.
60. The ear tube of clause 50, wherein the ear tube comprises a cross-sectional profile that is uniform throughout the ear tube.
61. The ear tube of clause 60, wherein the uniform cross-sectional profile is determined by at least one of a visual inspection, scanning electron microscopy, fluorescence microscopy, DSC, TEM, XRD, EPMA, or FTIR.
62. The ear tube of clause 60, wherein the cross-sectional profile relates to at least one of a density, an absence of voids, a consistent pattern, or a chemical profile.
63. The ear tube of clause 50, wherein a main tube of the ear tube has an axial length at least several times its diameter.
64. The ear tube of clause 50, wherein a main tube of the ear tube has an inner diameter of between 0.1 and 2.9 mm, between 0.1 and 2.5 mm, between 0.1 and 2 mm, between 0.1 and 1.5 mm, between 0.1 and 1 mm, between 1.0 and 3 mm, between 1.0 and 2 mm, or between 1.0 and 1.5 mm, including but not limited to, at least 0.1 mm, at least 0.5 mm, at least 0.6 mm, at least 0.7 mm. at least 0.8 mm, at least 0.9 mm, at least 1.0 mm, at least 1.1 mm, at least 1.2 mm, at least 1.3 mm, at least 1.4 mm, at least 1.5 mm, at least 2.0 mm, at least 2.5 mm, at least 3.0 mm, or at least 3.8 mm.
65. The ear tube of clause 50, wherein a main tube of the ear tube has an outer diameter of between 0.1 and 2.9 mm, between 0.1 and 2.5 mm, between 0.1 and 2 mm, between 0.1 and 1.5 mm, between 0.1 and 1 mm, between 1.0 and 3 mm, between 1.0 and 2 mm, or between 1.0 and 1.5 mm, including but not limited to, at least 0.1 mm, at least 0.5 mm, at least 0.6 mm, at least 0.7 mm. at least 0.8 mm, at least 0.9 mm, at least 1.0 mm, at least 1.1 mm, at least 1.2 mm, at least 1.3 mm, at least 1.4 mm, at least 1.5 mm, at least 2.0 mm, at least 2.5 mm, at least 3.0 mm, or at least 3.8 mm.
66. The ear tube of clause 50, wherein a diameter of an inner flange is between 1.0 mm and 10.0 mm, between 2.0 mm and 9.0 mm, between 2.0 mm and 7.0 mm, or between 3.0 mm and 6.0 mm, including but not limited to, at least 1 mm, at least 1.5 mm, at least 2.0 mm, at least 3.0 mm. at least 4.0 mm, at least 5.0 mm, at least 6.0 mm, at least 7.0 mm, at least 8.0 mm, at least 9.0 mm, or at least 10.0 mm.
67. The ear tube of clause 50, wherein a diameter of an outer flange is between 1.0 mm and 10.0 mm, between 2.0 mm and 9.0 mm, between 2.0 mm and 7.0 mm, or between 3.0 mm and 6.0 mm, including but not limited to, at least 1 mm, at least 1.5 mm, at least 2.0 mm, at least 3.0 mm. at least 4.0 mm, at least 5.0 mm, at least 6.0 mm, at least 7.0 mm, at least 8.0 mm, at least 9.0 mm, or at least 10.0 mm.
68. The ear tube of clause 50, wherein a wall of a main tube has a thickness of between 0.1 mm and 0.5 mm, between 0.1 mm and 0.4 mm, or between 0.3 mm and 0.5 mm, including but not limited to, at least 0.1 mm, at least 0.2 mm, at least 0.3 mm, at least 0.4 mm, or at least 0.5 mm.
69. The ear tube of clause 50, wherein a distance between a top surface of an inner flange and a bottom surface of an outer flange is between 0.05 mm and 12.0 mm, between 0.1 mm and 7.5 mm, between 1 mm and 7.5 mm, between 2.0 mm and 5.0 mm, including but not limited to, at least 0.05 mm, at least 0.1 mm, at least 0.5 mm, at least 1.0 mm, at least 2.0 mm. at least 3.0 mm, at least 4.0 mm, at least 5.0 mm, at least 7.5 mm, at least 10.0 mm, or at least 12.0 mm.
70. The ear tube of clause 50, wherein a total length of the ear tube is between 0.5 and 3 mm, between 0.5 and 2.5 mm, between 1 and 2.5 mm, between 1 and 2 mm, between 1 and 1.5 mm, between 2.0 mm and 5.0 mm, or between 3.0 mm and 8.0 mm, including but not limited to, at least 1.5 mm, at least 2.0 mm. at least 3.0 mm, at least 4.0 mm, at least 5.0 mm, at least 7.5 mm, or at least 8.0 mm.
71. The ear tube of clause 50, wherein an outer flange and an inner flange of the ear tube are formed from a same material.
72. The ear tube of clause 50, wherein an outer flange and an inner flange of the ear tube are formed from different materials.
73. The ear tube of clause 50, wherein an outer flange and an inner flange of the ear tube are formed from at least two biopolymers, each of the at least two biopolymers defining a thickness of the flange.
74. The ear tube of clause 50, wherein a main tube is formed from a single biopolymer.
75. The ear tube of clause 50, wherein a main tube is formed from a plurality of biopolymers, wherein a first biopolymer of the plurality of biopolymers forms an inner surface of the main tube and a second biopolymer of the plurality of biopolymers is disposed circumferentially around the first biopolymer and forms an outer surface of the main tube.
76. The ear tube of clause 50, wherein the ear tube comprises a first biopolymer forming at least a portion of a main tube of the ear tube and at least a portion of at least one flange, and a second biopolymer forming at least a portion of the main tube and at least a portion of at least flange.
77. The ear tube of clause 50, wherein an inner flange of the ear tube comprises chitosan and a main tube of the ear tube and an outer flange comprise silk.
78. The ear tube of clause 50, wherein the at least one material is silk fibroin.
79. The ear tube of clause 50, wherein the at least one material is at least one of silk fibroin or chitosan polysaccharide.
80. The ear tube of clause 50, wherein the ear tube comprises silk fibroin and chitosan polysaccharide.
81. The ear tube of clause 50, wherein the ear tube comprises silk fibroin and alginate.
82. The ear tube of clause 50, wherein the at least one material is an aqueous silk solution, an aqueous alginate solution, an aqueous fibrinogen solution, an aqueous chitosan solution, or an aqueous collagen solution.
83. The ear tube of clause 50, wherein the ear tube is formed via centrifugal molding.
84. The ear tube of clause 50, wherein the ear tube is formed via thermal molding and machining.
85. The ear tube of clause 50, wherein the ear tube is formed via centrifugal molding, thermal molding, and machining.
86. The ear tube of clause 50, wherein the material is at least one biopolymer and is assembled via an aqueous-based polymerization.
87. The ear tube of clause 50, wherein the ear tube comprises at least one additive.
88. The ear tube of clause 87, wherein the at least one additive is at least one of a bioactive molecule, a hydrophilic plasticizer, a hydrophobic plasticizer, a pigment, or a dye.
89. The ear tube of clause 88, wherein the bioactive molecule is an active pharmaceutical ingredient, an antibiotic, an anti-inflammatory agent, a steroid, a growth factor, or an enzyme.
90. The ear tube of clause 87, wherein the at least one additive is dip-coated onto the ear tube.
91. The ear tube of clause 90, wherein the dip-coated ear tube is subjected to water annealing.
92. The ear tube of clause 87, wherein the additive is dissolved in a biopolymer solution before dip-coating.
93. The ear tube of clause 87, wherein the ear tube is impregnated with the at least one additive.
94. The ear tube of clause 89, wherein the enzyme is at least one of a proteolytic enzyme, a serine protease, protease XIV, proteinase K. alpha-chymotrypsin, collagenase, MMP-1, MMP-2, or glucose oxidase.
95. The ear tube of clause 50, wherein the ear tube is designed to degrade on demand.
96. The ear tube of clause 95, wherein degradation commences when the ear tube is exposed to a solvent.
97. The ear tube of clause 96 wherein the solvent is at least one of a glucose solution, hydrogen peroxide, carbamide peroxide, an alcohol, a calcium chelator, or EDTA.
98. The ear tube of clause 50, wherein the ear tube is a tympanostomy tube.
99. An ear tube, comprising:
   a hollow cylindrical body having at least one flanged end, wherein the ear tube comprises at least one biopolymer, the ear tube having at least one characteristic centrifugal property selected from the group consisting of:
   i) a uniform density across at least a portion of the ear tube;
   ii) at least a portion of the ear tube is optically nontransparent;
   iii) at least a portion of the ear tube is free of exterior layering;
   iv) at least a portion of the ear tube is free of interior layering;
   v) at least a portion of the ear tube is interior-void-free;
   vi) at least a portion of the ear tube is free of cylindrical extrusion gaps; and
   vii) at least a portion of the ear tube is free of layering.
100. The ear tube of clause 99, wherein at least one of an outer flange or an inner flange is angled.
101. The ear tube of clause 100, wherein the at least one of the outer flange or inner flange defines a plane which is inclined at an angle with respect to a plane disposed perpendicular to an axis of the hollow cylindrical body.
102. The ear tube of clause 101, wherein the angle is between 15 degrees and 45 degrees, including but not limited to, at least 15 degrees, at least 30 degrees, or at least 45 degrees.
103. The ear tube of clause 100, wherein at least one of the outer flange or inner flange comprises an insertion protuberance.
104. The ear tube of clause 99, wherein the at least one flanged end comprises an outer flange and an inner flange of the ear tube that are at least one of same or different with respect to at least one of a thickness, a relative angle, a shape, an orientation, or a geometry.
105. The ear tube of clause 99, wherein the hollow cylindrical body has a uniform diameter.
106. The ear tube of clause 99, wherein the hollow cylindrical body has a non-uniform diameter along its length.
107. The ear tube of clause 99, wherein the hollow cylindrical body tapers along its length.
108. The ear tube of clause 99, wherein the ear tube has a uniform density throughout.
109. The ear tube of clause 99, wherein the ear tube comprises a cross-sectional profile that is uniform throughout the ear tube.
110. The ear tube of clause 109, wherein the uniform cross-sectional profile is determined by at least one of a visual inspection, scanning electron microscopy, fluorescence microscopy, DSC, TEM, XRD, EPMA, or FTIR.
111. The ear tube of clause 109, wherein the cross-sectional profile relates to at least one of a density, an absence of voids, a consistent pattern, or a chemical profile.
112. The ear tube of clause 99, wherein the hollow cylindrical body has an axial length at least several times its diameter.
113. The ear tube of clause 99, wherein the hollow cylindrical body has an inner diameter of between 0.1 and 2.9 mm, between 0.1 and 2.5 mm, between 0.1 and 2 mm, between 0.1 and 1.5 mm, between 0.1 and 1 mm, between 1.0 and 3 mm, between 1.0 and 2 mm, or between 1.0 and 1.5 mm, including but not limited to, at least 0.1 mm, at least 0.5 mm, at least 0.6 mm, at least 0.7 mm. at least 0.8 mm, at least 0.9 mm, at least 1.0 mm, at least 1.1 mm, at least 1.2 mm, at least 1.3 mm, at least 1.4 mm, at least 1.5 mm, at least 2.0 mm, at least 2.5 mm, at least 3.0 mm, or at least 3.8 mm.
114. The ear tube of clause 99, wherein the hollow cylindrical body has an outer diameter of between 0.1 and 2.9 mm, between 0.1 and 2.5 mm, between 0.1 and 2 mm, between 0.1 and 1.5 mm, between 0.1 and 1 mm, between 1.0 and 3 mm, between 1.0 and 2 mm, or between 1.0 and 1.5 mm, including but not limited to, at least 0.1 mm, at least 0.5 mm, at least 0.6 mm, at least 0.7 mm. at least 0.8 mm, at least 0.9 mm, at least 1.0 mm, at least 1.1 mm, at least 1.2 mm, at least 1.3 mm, at least 1.4 mm, at least 1.5 mm, at least 2.0 mm, at least 2.5 mm, at least 3.0 mm, or at least 3.8 mm.
115. The ear tube of clause 99, wherein the inner flange diameter is between 1.0 mm and 10.0 mm, between 2.0 mm and 9.0 mm, between 2.0 mm and 7.0 mm, or between 3.0 mm and 6.0 mm, including but not limited to, at least 1 mm, at least 1.5 mm, at least 2.0 mm, at least 3.0 mm. at least 4.0 mm, at least 5.0 mm, at least 6.0 mm, at least 7.0 mm, at least 8.0 mm, at least 9.0 mm, or at least 10.0 mm.
116. The ear tube of clause 99, wherein the outer flange diameter is between 1.0 mm and 10.0 mm, between 2.0 mm and 9.0 mm, between 2.0 mm and 7.0 mm, or between 3.0 mm and 6.0 mm, including but not limited to, at least 1 mm, at least 1.5 mm, at least 2.0 mm, at least 3.0 mm. at least 4.0 mm, at least 5.0 mm, at least 6.0 mm, at least 7.0 mm, at least 8.0 mm, at least 9.0 mm, or at least 10.0 mm.
117. The ear tube of clause 99, wherein a wall of the hollow cylindrical body has a thickness of between 0.1 mm and 0.5 mm, between 0.1 mm and 0.4 mm, or between 0.3 mm and 0.5 mm, including but not limited to, at least 0.1 mm, at least 0.2 mm, at least 0.3 mm, at least 0.4 mm, or at least 0.5 mm.
118. The ear tube of clause 99, wherein a distance between a top surface of the inner flange and a bottom surface of the outer flange is between 0.05 mm and 12.0 mm, between 0.1 mm and 7.5 mm, between 1 mm and 7.5 mm, between 2.0 mm and 5.0 mm, including but not limited to, at least 0.05 mm, at least 0.1 mm, at least 0.5 mm, at least 1.0 mm, at least 2.0 mm. at least 3.0 mm, at least 4.0 mm, at least 5.0 mm, at least 7.5 mm, at least 10.0 mm, or at least 12.0 mm.
119. The ear tube of clause 99, wherein a total length of the ear tube is between 0.5 and 3 mm, between 0.5 and 2.5 mm, between 1 and 2.5 mm, between 1 and 2 mm, between 1 and 1.5 mm, between 2.0 mm and 5.0 mm, or between 3.0 mm and 8.0 mm, including but not limited to, at least 1.5 mm, at least 2.0 mm. at least 3.0 mm, at least 4.0 mm, at least 5.0 mm, at least 7.5 mm, or at least 8.0 mm.
120. The ear tube of clause 99, wherein the outer flange and the inner flange are formed from the same biopolymer.
121. The ear tube of clause 99, wherein the outer flange and the inner flange are formed from different biopolymers.
122. The ear tube of clause 99, wherein an outer flange and an inner flange are formed from at least two biopolymers, each of the at least two biopolymers defining a thickness of the flange.
123. The ear tube of clause 99, wherein the hollow cylindrical body is formed from a single biopolymer.
124. The ear tube of clause 99, wherein the hollow cylindrical body is formed from a plurality of biopolymers, wherein a first biopolymer of the plurality of biopolymers forms an inner surface of the hollow cylindrical body and a second biopolymer of the plurality of biopolymers is disposed circumferentially around the first biopolymer and forms an outer surface of the hollow cylindrical body.
125. The ear tube of clause 99, wherein the ear tube comprises a first biopolymer forming at least a portion of the main tube and at least a portion of at least one of the flanges, and a second biopolymer forming at least a portion of the hollow cylindrical body and at least a portion of at least one of the flanges.
126. The ear tube of clause 99, wherein an inner flange comprises chitosan and the hollow cylindrical body and an outer flange comprise silk.
127. The ear tube of clause 99, wherein the at least one biopolymer is silk fibroin.
128. The ear tube of clause 99, wherein the at least one biopolymer is at least one of silk fibroin or chitosan polysaccharide.
129. The ear tube of clause 99, wherein the ear tube comprises silk fibroin and chitosan polysaccharide.
130. The ear tube of clause 99, wherein the ear tube comprises silk fibroin and alginate.
131. The ear tube of clause 99, wherein the at least one biopolymer is an aqueous silk solution, an aqueous alginate solution, an aqueous fibrinogen solution, an aqueous chitosan solution, or an aqueous collagen solution.
132. The ear tube of clause 99, wherein the ear tube is formed via centrifugal molding.
133. The ear tube of clause 99, wherein the ear tube is formed via thermal molding and machining.
134. The ear tube of clause 99, wherein the ear tube is formed via centrifugal molding, thermal molding, and machining.
135. The ear tube of clause 99, wherein the at least one biopolymer is assembled via an aqueous-based polymerization.
136. The ear tube of clause 99, wherein the ear tube comprises at least one additive.
137. The ear tube of clause 136, wherein the at least one additive is at least one of a bioactive molecule, a hydrophilic plasticizer, a hydrophobic plasticizer, a pigment, or a dye.
138. The ear tube of clause 137, wherein the bioactive molecule is an active pharmaceutical ingredient, an antibiotic, an anti-inflammatory agent, a steroid, a growth factor, or an enzyme.
139. The ear tube of clause 136, wherein the at least one additive is dip-coated onto the ear tube.
140. The ear tube of clause 139, wherein the dip-coated ear tube is subjected to water annealing.
141. The ear tube of clause 136, wherein the additive is dissolved in a biopolymer solution before dip-coating.
142. The ear tube of clause 136, wherein the ear tube is impregnated with the at least one additive.
143. The ear tube of clause 138, wherein the enzyme is at least one of a proteolytic enzyme, a serine protease, protease XIV, proteinase K. alpha-chymotrypsin, collagenase, MMP-1, MMP-2, or glucose oxidase.
144. The ear tube of clause 99, wherein the ear tube is designed to degrade on demand.
145. The ear tube of clause 144, wherein degradation commences when the ear tube is exposed to a solvent.
146. The ear tube of clause 145 wherein the solvent is at least one of a glucose solution, hydrogen peroxide, carbamide peroxide, an alcohol, a calcium chelator, or EDTA.
147. The ear tube of clause 99, wherein the ear tube is a tympanostomy tube.
148. An ear tube, comprising:
   a main tube, the main tube extending along a center axis defining a conduit for fluid communication, the main tube having a first end and a second end; and
   an outer flange extending radially away from the main tube on the first end, wherein the ear tube comprises at least one biopolymer, the ear tube having at least one characteristic centrifugal property selected from the group consisting of:
   i) a uniform density across at least a portion of the ear tube;
   ii) at least a portion of the ear tube is optically nontransparent;
   iii) at least a portion of the ear tube is free of exterior layering;
   iv) at least a portion of the ear tube is free of interior layering;
   v) at least a portion of the ear tube is interior-void-free;
   vi) at least a portion of the ear tube is free of cylindrical extrusion gaps; and
   vii) at least a portion of the ear tube is free of layering.
149. The ear tube of clause 148, wherein at least one of the outer flange or inner flange is angled.
150. The ear tube of clause 149, wherein the at least one of the outer flange or inner flange defines a plane which is inclined at an angle with respect to a plane disposed perpendicular to the axis of the main tube.
151. The ear tube of clause 150, wherein the angle is between 15 degrees and 45 degrees, including but not limited to, at least 15 degrees, at least 30 degrees, or at least 45 degrees.
152. The ear tube of clause 149, wherein at least one of the outer flange or inner flange comprises an insertion protuberance.
153. The ear tube of clause 148, wherein the outer flange and an optional inner flange of the ear tube are at least one of same or different with respect to at least one of a thickness, a relative angle, a shape, an orientation, or a geometry.
154. The ear tube of clause 148, wherein the ear tube has a uniform diameter throughout a main tube.
155. The ear tube of clause 148, wherein the ear tube has a non-uniform diameter along a length of the main tube.
156. The ear tube of clause 148, wherein the ear tube tapers along a length of the main tube.
157. The ear tube of clause 148, wherein the ear tube has a uniform density throughout.
158. The ear tube of clause 148, wherein the ear tube comprises a cross-sectional profile that is uniform throughout the ear tube.
159. The ear tube of clause 158, wherein the uniform cross-sectional profile is determined by at least one of a visual inspection, scanning electron microscopy, fluorescence microscopy, DSC, TEM, XRD, EPMA, or FTIR.
160. The ear tube of clause 158, wherein the cross-sectional profile relates to at least one of a density, an absence of voids, a consistent pattern, or a chemical profile.
161. The ear tube of clause 148, wherein the main tube has an axial length at least several times its diameter.
162. The ear tube of clause 148, wherein the main tube has an inner diameter of between 0.1 and 2.9 mm, between 0.1 and 2.5 mm, between 0.1 and 2 mm, between 0.1 and 1.5 mm, between 0.1 and 1 mm, between 1.0 and 3 mm, between 1.0 and 2 mm, or between 1.0 and 1.5 mm, including but not limited to, at least 0.1 mm, at least 0.5 mm, at least 0.6 mm, at least 0.7 mm. at least 0.8 mm, at least 0.9 mm, at least 1.0 mm, at least 1.1 mm, at least 1.2 mm, at least 1.3 mm, at least 1.4 mm, at least 1.5 mm, at least 2.0 mm, at least 2.5 mm, at least 3.0 mm, or at least 3.8 mm.
163. The ear tube of clause 148, wherein the main tube has an outer diameter of between 0.1 and 2.9 mm, between 0.1 and 2.5 mm, between 0.1 and 2 mm, between 0.1 and 1.5 mm, between 0.1 and 1 mm, between 1.0 and 3 mm, between 1.0 and 2 mm, or between 1.0 and 1.5 mm, including but not limited to, at least 0.1 mm, at least 0.5 mm, at least 0.6 mm, at least 0.7 mm. at least 0.8 mm, at least 0.9 mm, at least 1.0 mm, at least 1.1 mm, at least 1.2 mm, at least 1.3 mm, at least 1.4 mm, at least 1.5 mm, at least 2.0 mm, at least 2.5 mm, at least 3.0 mm, or at least 3.8 mm.
164. The ear tube of clause 148, wherein the inner flange diameter is between 1.0 mm and 10.0 mm, between 2.0 mm and 9.0 mm, between 2.0 mm and 7.0 mm, or between 3.0 mm and 6.0 mm, including but not limited to, at least 1 mm, at least 1.5 mm, at least 2.0 mm, at least 3.0 mm. at least 4.0 mm, at least 5.0 mm, at least 6.0 mm, at least 7.0 mm, at least 8.0 mm, at least 9.0 mm, or at least 10.0 mm.
165. The ear tube of clause 148, wherein the outer flange diameter is between 1.0 mm and 10.0 mm, between 2.0 mm and 9.0 mm, between 2.0 mm and 7.0 mm, or between 3.0 mm and 6.0 mm, including but not limited to, at least 1 mm, at least 1.5 mm, at least 2.0 mm, at least 3.0 mm. at least 4.0 mm, at least 5.0 mm, at least 6.0 mm, at least 7.0 mm, at least 8.0 mm, at least 9.0 mm, or at least 10.0 mm.
166. The ear tube of clause 148, wherein a wall of the main tube has a thickness of between 0.1 mm and 0.5 mm, between 0.1 mm and 0.4 mm, or between 0.3 mm and 0.5 mm, including but not limited to, at least 0.1 mm, at least 0.2 mm, at least 0.3 mm, at least 0.4 mm, or at least 0.5
167. The ear tube of clause 148, wherein a distance between a top surface of an optional inner flange and a bottom surface of the outer flange is between 0.05 mm and 12.0 mm, between 0.1 mm and 7.5 mm, between 1 mm and 7.5 mm, between 2.0 mm and 5.0 mm, including but not limited to, at least 0.05 mm, at least 0.1 mm, at least 0.5 mm, at least 1.0 mm, at least 2.0 mm. at least 3.0 mm, at least 4.0 mm, at least 5.0 mm, at least 7.5 mm, at least 10.0 mm, or at least 12.0 mm.
168. The ear tube of clause 148, wherein a total length of the ear tube is between 0.5 and 3 mm, between 0.5 and 2.5 mm, between 1 and 2.5 mm, between 1 and 2 mm, between 1 and 1.5 mm, between 2.0 mm and 5.0 mm, or between 3.0 mm and 8.0 mm, including but not limited to, at least 1.5 mm, at least 2.0 mm. at least 3.0 mm, at least 4.0 mm, at least 5.0 mm, at least 7.5 mm, or at least 8.0 mm.
169. The ear tube of clause 148, wherein the outer flange and an optional inner flange are formed from a same biopolymer.
170. The ear tube of clause 148, wherein the outer flange and an optional inner flange are formed from different biopolymers.
171. The ear tube of clause 148, wherein the outer flange and the inner flange are formed from at least two biopolymers, each of the at least two biopolymers defining a thickness of the flange.
172. The ear tube of clause 148, wherein the main tube is formed from a single biopolymer.
173. The ear tube of clause 148, wherein the main tube is formed from a plurality of biopolymers, wherein a first biopolymer of the plurality of biopolymers forms an inner surface of the main tube and a second biopolymer of the plurality of biopolymers is disposed circumferentially around the first biopolymer and forms an outer surface of the main tube.
174. The ear tube of clause 148, wherein the ear tube comprises a first biopolymer forming at least a portion of the main tube and at least a portion of at least one of the flanges, and a second biopolymer forming at least a portion of the main tube and at least a portion of at least one of the flanges.
175. The ear tube of clause 148, wherein an optional inner flange comprises chitosan and the main tube and outer flange comprise silk.
176. The ear tube of clause 148, wherein the at least one biopolymer is silk fibroin.
177. The ear tube of clause 148, wherein the at least one biopolymer is at least one of silk fibroin or chitosan polysaccharide.
178. The ear tube of clause 148, wherein the ear tube comprises silk fibroin and chitosan polysaccharide.
179. The ear tube of clause 148, wherein the ear tube comprises silk fibroin and alginate.
180. The ear tube of clause 148, wherein the at least one biopolymer is an aqueous silk solution, an aqueous alginate solution, an aqueous fibrinogen solution, an aqueous chitosan solution, or an aqueous collagen solution.
181. The ear tube of clause 148, wherein the ear tube is formed via centrifugal molding.
182. The ear tube of clause 148, wherein the ear tube is formed via thermal molding and machining.
183. The ear tube of clause 148, wherein the ear tube is formed via centrifugal molding, thermal molding, and machining.
184. The ear tube of clause 148, wherein the at least one biopolymer is assembled via an aqueous-based polymerization.
185. The ear tube of clause 148, wherein the ear tube comprises at least one additive.
186. The ear tube of clause 185, wherein the at least one additive is at least one of a bioactive molecule, a hydrophilic plasticizer, a hydrophobic plasticizer, a pigment, or a dye.
187. The ear tube of clause 186, wherein the bioactive molecule is an active pharmaceutical ingredient, an antibiotic, an anti-inflammatory agent, a steroid, a growth factor, or an enzyme.
188. The ear tube of clause 185, wherein the at least one additive is dip-coated onto the ear tube.
189. The ear tube of clause 188, wherein the dip-coated ear tube is subjected to water annealing.
190. The ear tube of clause 185, wherein the additive is dissolved in a biopolymer solution before dip-coating.
191. The ear tube of clause 185, wherein the ear tube is impregnated with the at least one additive.
192. The ear tube of clause 187, wherein the enzyme is at least one of a proteolytic enzyme, a serine protease, protease XIV, proteinase K. alpha-chymotrypsin, collagenase, MMP-1, MMP-2, or glucose oxidase.
193. The ear tube of clause 148, wherein the ear tube is designed to degrade on demand.
194. The ear tube of clause 193, wherein degradation commences when the ear tube is exposed to a solvent.
195. The ear tube of clause 194 wherein the solvent is at least one of a glucose solution, hydrogen peroxide, carbamide peroxide, an alcohol, a calcium chelator, or EDTA.
196. The ear tube of clause 148, wherein the ear tube is a tympanostomy tube.
197. An ear tube, comprising:
   a main tube, the main tube extending along a center axis defining a conduit for fluid communication, the main tube having a first end and a second end;
   an outer flange extending radially away from the main tube on a first end;
   an inner flange extending radially away from the main tube on a second end; and
   a frustoconical portion of the main tube extending longitudinally from at least one of the first end or the second end, the frustoconical portion extending beyond a width of the flange on the at least one of the first end or the second end, wherein the ear tube comprises at least one biopolymer, the ear tube having at least one characteristic centrifugal property selected from the group consisting of:
   i) a uniform density across at least a portion of the ear tube;
   ii) at least a portion of the ear tube is optically nontransparent;
   iii) at least a portion of the ear tube is free of exterior layering;
   iv) at least a portion of the ear tube is free of interior layering;
   v) at least a portion of the ear tube is interior-void-free;
   vi) at least a portion of the ear tube is free of cylindrical extrusion gaps; and
   vii) at least a portion of the ear tube is free of layering.
198. The ear tube of clause 197, wherein at least one of the outer flange or inner flange is angled.
199. The ear tube of clause 198, wherein the at least one of the outer flange or inner flange defines a plane which is inclined at an angle with respect to a plane disposed perpendicular to the axis of the main tube.
200. The ear tube of clause 199, wherein the angle is between 15 degrees and 45 degrees, including but not limited to, at least 15 degrees, at least 30 degrees, or at least 45 degrees.
201. The ear tube of clause 198, wherein at least one of the outer flange or inner flange comprises an insertion protuberance.
202. The ear tube of clause 197, wherein an outer flange and an inner flange of the ear tube are at least one of same or different with respect to at least one of a thickness, a relative angle, a shape, an orientation, or a geometry.
203. The ear tube of clause 197, wherein the ear tube has a uniform diameter throughout a main tube.
204. The ear tube of clause 197, wherein the ear tube has a non-uniform diameter along a length of the main tube.
205. The ear tube of clause 197, wherein the ear tube tapers along a length of the main tube.
206. The ear tube of clause 197, wherein the ear tube has a uniform density throughout.
207. The ear tube of clause 197, wherein the ear tube comprises a cross-sectional profile that is uniform throughout the ear tube.
208. The ear tube of clause 207, wherein the uniform cross-sectional profile is determined by at least one of a visual inspection, scanning electron microscopy, fluorescence microscopy, DSC, TEM, XRD, EPMA, or FTIR.
209. The ear tube of clause 207, wherein the cross-sectional profile relates to at least one of a density, an absence of voids, a consistent pattern, or a chemical profile.
210. The ear tube of clause 197, wherein the main tube has an axial length at least several times its diameter.
211. The ear tube of clause 197, wherein the main tube has an inner diameter of between 0.1 and 2.9 mm, between 0.1 and 2.5 mm, between 0.1 and 2 mm, between 0.1 and 1.5 mm, between 0.1 and 1 mm, between 1.0 and 3 mm, between 1.0 and 2 mm, or between 1.0 and 1.5 mm, including but not limited to, at least 0.1 mm, at least 0.5 mm, at least 0.6 mm, at least 0.7 mm. at least 0.8 mm, at least 0.9 mm, at least 1.0 mm, at least 1.1 mm, at least 1.2 mm, at least 1.3 mm, at least 1.4 mm, at least 1.5 mm, at least 2.0 mm, at least 2.5 mm, at least 3.0 mm, or at least 3.8 mm.
212. The ear tube of clause 197, wherein the main tube has an outer diameter of between 0.1 and 2.9 mm, between 0.1 and 2.5 mm, between 0.1 and 2 mm, between 0.1 and 1.5 mm, between 0.1 and 1 mm, between 1.0 and 3 mm, between 1.0 and 2 mm, or between 1.0 and 1.5 mm, including but not limited to, at least 0.1 mm, at least 0.5 mm, at least 0.6 mm, at least 0.7 mm. at least 0.8 mm, at least 0.9 mm, at least 1.0 mm, at least 1.1 mm, at least 1.2 mm, at least 1.3 mm, at least 1.4 mm, at least 1.5 mm, at least 2.0 mm, at least 2.5 mm, at least 3.0 mm, or at least 3.8 mm.
213. The ear tube of clause 197, wherein the inner flange diameter is between 1.0 mm and 10.0 mm, between 2.0 mm and 9.0 mm, between 2.0 mm and 7.0 mm, or between 3.0 mm and 6.0 mm, including but not limited to, at least 1 mm, at least 1.5 mm, at least 2.0 mm, at least 3.0 mm. at least 4.0 mm, at least 5.0 mm, at least 6.0 mm, at least 7.0 mm, at least 8.0 mm, at least 9.0 mm, or at least 10.0 mm.
214. The ear tube of clause 197, wherein the outer flange diameter is between 1.0 mm and 10.0 mm, between 2.0 mm and 9.0 mm, between 2.0 mm and 7.0 mm, or between 3.0 mm and 6.0 mm, including but not limited to, at least 1 mm, at least 1.5 mm, at least 2.0 mm, at least 3.0 mm. at least 4.0 mm, at least 5.0 mm, at least 6.0 mm, at least 7.0 mm, at least 8.0 mm, at least 9.0 mm, or at least 10.0 mm.
215. The ear tube of clause 197, wherein a wall of the main tube has a thickness of between 0.1 mm and 0.5 mm, between 0.1 mm and 0.4 mm, or between 0.3 mm and 0.5 mm, including but not limited to, at least 0.1 mm, at least 0.2 mm, at least 0.3 mm, at least 0.4 mm, or at least 0.5
216. The ear tube of clause 197, wherein a distance between a top surface of the inner flange and a bottom surface of the outer flange is between 0.05 mm and 12.0 mm, between 0.1 mm and 7.5 mm, between 1 mm and 7.5 mm, between 2.0 mm and 5.0 mm, including but not limited to, at least 0.05 mm, at least 0.1 mm, at least 0.5 mm, at least 1.0 mm, at least 2.0 mm. at least 3.0 mm, at least 4.0 mm, at least 5.0 mm, at least 7.5 mm, at least 10.0 mm, or at least 12.0 mm.
217. The ear tube of clause 197, wherein a total length of the ear tube is between 0.5 and 3 mm, between 0.5 and 2.5 mm, between 1 and 2.5 mm, between 1 and 2 mm, between 1 and 1.5 mm, between 2.0 mm and 5.0 mm, or between 3.0 mm and 8.0 mm, including but not limited to, at least 1.5 mm, at least 2.0 mm. at least 3.0 mm, at least 4.0 mm, at least 5.0 mm, at least 7.5 mm, or at least 8.0 mm.
218. The ear tube of clause 197, wherein the outer flange and the inner flange are formed from a same biopolymer.
219. The ear tube of clause 197, wherein the outer flange and the inner flange are formed from different biopolymers.
220. The ear tube of clause 197, wherein the outer flange and the inner flange are formed from at least two biopolymers, each of the at least two biopolymers defining a thickness of the flange.
221. The ear tube of clause 197, wherein the main tube is formed from a single biopolymer.
222. The ear tube of clause 197, wherein the main tube is formed from a plurality of biopolymers, wherein a first biopolymer of the plurality of biopolymers forms an inner surface of the main tube and a second biopolymer of the plurality of biopolymers is disposed circumferentially around the first biopolymer and forms an outer surface of the main tube.
223. The ear tube of clause 197, wherein the ear tube comprises a first biopolymer forming at least a portion of the main tube and at least a portion of at least one of the flanges, and a second biopolymer forming at least a portion of the main tube and at least a portion of at least one of the flanges.
224. The ear tube of clause 197, wherein the inner flange comprises chitosan and the main tube and outer flange comprise silk.
225. The ear tube of clause 197, wherein the at least one biopolymer is silk fibroin.
226. The ear tube of clause 197, wherein the at least one biopolymer is at least one of silk fibroin or chitosan polysaccharide.
227. The ear tube of clause 197, wherein the ear tube comprises silk fibroin and chitosan polysaccharide.
228. The ear tube of clause 197, wherein the ear tube comprises silk fibroin and alginate.
229. The ear tube of clause 197, wherein the at least one biopolymer is an aqueous silk solution, an aqueous alginate solution, an aqueous fibrinogen solution, an aqueous chitosan solution, or an aqueous collagen solution.
230. The ear tube of clause 197, wherein the ear tube is formed via centrifugal molding.
231. The ear tube of clause 197, wherein the ear tube is formed via thermal molding and machining.
232. The ear tube of clause 197, wherein the ear tube is formed via centrifugal molding, thermal molding, and machining.
233. The ear tube of clause 197, wherein the at least one biopolymer is assembled via an aqueous-based polymerization.
234. The ear tube of clause 197, wherein the ear tube comprises at least one additive.
235. The ear tube of clause 234, wherein the at least one additive is at least one of a bioactive molecule, a hydrophilic plasticizer, a hydrophobic plasticizer, a pigment, or a dye.
236. The ear tube of clause 235, wherein the bioactive molecule is an active pharmaceutical ingredient, an antibiotic, an anti-inflammatory agent, a steroid, a growth factor, or an enzyme.
237. The ear tube of clause 234, wherein the at least one additive is dip-coated onto the ear tube.
238. The ear tube of clause 237, wherein the dip-coated ear tube is subjected to water annealing.
239. The ear tube of clause 234, wherein the additive is dissolved in a biopolymer solution before dip-coating.
240. The ear tube of clause 234, wherein the ear tube is impregnated with the at least one additive.
241. The ear tube of clause 236, wherein the enzyme is at least one of a proteolytic enzyme, a serine protease, protease XIV, proteinase K. alpha-chymotrypsin, collagenase, MMP-1, MMP-2, or glucose oxidase.
242. The ear tube of clause 197, wherein the ear tube is designed to degrade on demand.
243. The ear tube of clause 242, wherein degradation commences when the ear tube is exposed to a solvent.
244. The ear tube of clause 243 wherein the solvent is at least one of a glucose solution, hydrogen peroxide, carbamide peroxide, an alcohol, a calcium chelator, or EDTA.
245. The ear tube of clause 197, wherein the ear tube is a tympanostomy tube.
246. A method of forming an ear tube, comprising:
   depositing at least one aqueous biopolymer solution into an ear tube mold;
   subjecting the ear tube mold and its contents to a first centrifugation;
   depositing a polymerization bath into the ear tube mold and subjecting the ear tube mold and its contents to a second centrifugation to polymerize the aqueous biopolymer solution into a shape of an ear tube; and
   drying the ear tube.
247. The method of clause 246, wherein the ear tube dwells in the mold after the second centrifugation to complete polymerization before the drying.
248. The method of clause 246, wherein the at least one aqueous biopolymer solution is an aqueous silk solution, an aqueous alginate solution, an aqueous fibrinogen solution, an aqueous chitosan solution, or an aqueous collagen solution.
249. The method of clause 246, further comprising subjecting the ear tube to at least one of cross-linking, dipcoating, covalent bonding, heat treatment, pressure treatment, water annealing, sterilization, surface modifications, chemical etching, chemical smoothing, or physical smoothing.
250. A method of degrading an ear tube, comprising:
   exposing an ear tube to a solvent,
wherein the ear tube comprises at least one biopolymer having at least one characteristic centrifugal property selected from the group consisting of:
   i) a uniform density across at least a portion of the ear tube;
   ii) at least a portion of the ear tube is optically nontransparent;
   iii) at least a portion of the ear tube is free of exterior layering;
   iv) at least a portion of the ear tube is free of interior layering;
   v) at least a portion of the ear tube is interior-void-free;
   vi) at least a portion of the ear tube is free of cylindrical extrusion gaps; and
   vii) at least a portion of the ear tube is free of layering.
251. The method of clause 250, wherein the solvent is at least one of a glucose solution, hydrogen peroxide, carbamide peroxide, an alcohol, a calcium chelator, or EDTA.
252. The method of clause 250, wherein exposing is over a period of at least one of minutes, hours, or days.
253. The ear tube or method of any one of the preceding clauses, comprising a uniform density across the at least a portion of the ear tube.
254. The ear tube or method of any one of the preceding clauses, wherein the at least a portion of the ear tube is optically nontransparent.
255. The ear tube or method of any one of the preceding clauses, wherein the at least a portion of the ear tube is free of exterior layering.
256. The ear tube or method of any one of the preceding clauses, wherein the at least a portion of the ear tube is free of interior layering.
257. The ear tube or method of any one of the preceding clauses, wherein the at least a portion of the ear tube is interior-void-free.
258. The ear tube or method of any one of the preceding clauses, wherein the at least a portion of the ear tube is free of cylindrical extrusion gaps.
259. The ear tube or method of any one of the preceding clauses, wherein the at least a portion of the ear tube is free of layering.
260. An ear tube comprising chitosan, the ear tube having at least one characteristic centrifugal property selected from the group consisting of:
   i) a uniform density across at least a portion of the ear tube;
   ii) at least a portion of the ear tube is optically nontransparent;
   iii) at least a portion of the ear tube is free of exterior layering;
   iv) at least a portion of the ear tube is free of interior layering;
   v) at least a portion of the ear tube is interior-void-free;
   vi) at least a portion of the ear tube is free of cylindrical extrusion gaps; and
   vii) at least a portion of the ear tube is free of layering.
261. The ear tube of clause 260, wherein at least one of an outer flange or an inner flange of the ear tube is angled.
262. The ear tube of clause 261, wherein the at least one of the outer flange or inner flange defines a plane which is inclined at an angle with respect to a plane disposed perpendicular to an axis of a main tube of the ear tube.
263. The ear tube of clause 262, wherein the angle is between 15 degrees and 45 degrees, including but not limited to, at least 15 degrees, at least 30 degrees, or at least 45 degrees.
264. The ear tube of clause 261, wherein at least one of the outer flange or inner flange comprises an insertion protuberance.
265. The ear tube of clause 260, wherein an outer flange and an inner flange of the ear tube are at least one of same or different with respect to at least one of a thickness, a relative angle, a shape, an orientation, or a geometry.
266. The ear tube of clause 260, wherein the ear tube has a uniform diameter throughout its length.
267. The ear tube of clause 260, wherein the ear tube has a non-uniform diameter along its length.
268. The ear tube of clause 260, wherein the ear tube tapers along its length.
269. The ear tube of clause 260, wherein the ear tube has a uniform density throughout.
270. The ear tube of clause 260, wherein the ear tube comprises a cross-sectional profile that is uniform throughout the ear tube.
271. The ear tube of clause 270, wherein the uniform cross-sectional profile is determined by at least one of a visual inspection, scanning electron microscopy, fluorescence microscopy, DSC, TEM, XRD, EPMA, or FTIR.
272. The ear tube of clause 270, wherein the cross-sectional profile relates to at least one of a density, an absence of voids, a consistent pattern, or a chemical profile.
273. The ear tube of clause 260, wherein a main tube of the ear tube has an axial length at least several times its diameter.
274. The ear tube of clause 260, wherein a main tube of the ear tube has an inner diameter of between 0.1 and 2.9 mm, between 0.1 and 2.5 mm, between 0.1 and 2 mm, between 0.1 and 1.5 mm, between 0.1 and 1 mm, between 1.0 and 3 mm, between 1.0 and 2 mm, or between 1.0 and 1.5 mm, including but not limited to, at least 0.1 mm, at least 0.5 mm, at least 0.6 mm, at least 0.7 mm, at least 0.8 mm, at least 0.9 mm, at least 1.0 mm, at least 1.1 mm, at least 1.2 mm, at least 1.3 mm, at least 1.4 mm, at least 1.5 mm, at least 2.0 mm, at least 2.5 mm, at least 3.0 mm, or at least 3.8 mm.
275. The ear tube of clause 260, wherein a main tube of the ear tube has an outer diameter of between 0.1 and 2.9 mm, between 0.1 and 2.5 mm, between 0.1 and 2 mm, between 0.1 and 1.5 mm, between 0.1 and 1 mm, between 1.0 and 3 mm, between 1.0 and 2 mm, or between 1.0 and 1.5 mm, including but not limited to, at least 0.1 mm, at least 0.5 mm, at least 0.6 mm, at least 0.7 mm, at least 0.8 mm, at least 0.9 mm, at least 1.0 mm, at least 1.1 mm, at least 1.2 mm, at least 1.3 mm, at least 1.4 mm, at least 1.5 mm, at least 2.0 mm, at least 2.5 mm, at least 3.0 mm, or at least 3.8 mm.
276. The ear tube of clause 260, wherein the ear tube has an inner flange, the inner flange diameter is between 1.0 mm and 10.0 mm, between 2.0 mm and 9.0 mm, between 2.0 mm and 7.0 mm, or between 3.0 mm and 6.0 mm, including but not limited to, at least 1 mm, at least 1.5 mm, at least 2.0 mm, at least 3.0 mm, at least 4.0 mm, at least 5.0 mm, at least 6.0 mm, at least 7.0 mm, at least 8.0 mm, at least 9.0 mm, or at least 10.0 mm.
277. The ear tube of clause 260, wherein the ear tube has an outer flange, the outer flange diameter is between 1.0 mm and 10.0 mm, between 2.0 mm and 9.0 mm, between 2.0 mm and 7.0 mm, or between 3.0 mm and 6.0 mm, including but not limited to, at least 1 mm, at least 1.5 mm, at least 2.0 mm, at least 3.0 mm, at least 4.0 mm, at least 5.0 mm, at least 6.0 mm, at least 7.0 mm, at least 8.0 mm, at least 9.0 mm, or at least 10.0 mm.
278. The ear tube of clause 260, wherein a wall of a main tube of the ear tube has a thickness of between 0.1 mm and 0.5 mm, between 0.1 mm and 0.4 mm, or between 0.3 mm and 0.5 mm, including but not limited to, at least 0.1 mm, at least 0.2 mm, at least 0.3 mm, at least 0.4 mm, or at least 0.5 mm.
279. The ear tube of clause 260, wherein a distance between a top surface of an inner flange of the ear tube and a bottom surface of an outer flange of the ear tube is between 0.05 mm and 12.0 mm, between 0.1 mm and 7.5 mm, between 1 mm and 7.5 mm, between 2.0 mm and 5.0 mm, including but not limited to, at least 0.05 mm, at least 0.1 mm, at least 0.5 mm, at least 1.0 mm, at least 2.0 mm. at least 3.0 mm, at least 4.0 mm, at least 5.0 mm, at least 7.5 mm, at least 10.0 mm, or at least 12.0 mm.
280. The ear tube of clause 260, wherein a total length of the ear tube is between 0.5 and 3 mm, between 0.5 and 2.5 mm, between 1 and 2.5 mm, between 1 and 2 mm, between 1 and 1.5 mm, between 2.0 mm and 5.0 mm, or between 3.0 mm and 8.0 mm, including but not limited to, at least 1.5 mm, at least 2.0 mm. at least 3.0 mm, at least 4.0 mm, at least 5.0 mm, at least 7.5 mm, or at least 8.0 mm.
281. The ear tube of clause 260, wherein an outer flange and an inner flange of the ear tube are both formed from chitosan.
282. The ear tube of clause 260, wherein an outer flange and an inner flange of the ear tube are formed from different biopolymers, at least one of the biopolymers comprising chitosan.
283. The ear tube of clause 260, wherein an outer flange and an inner flange of the ear tube are formed from at least two biopolymers, each of the at least two biopolymers defining a thickness of the flange.
284. The ear tube of clause 260, wherein a main tube of the ear tube is formed from chitosan.
285. The ear tube of clause 260, wherein a main tube of the ear tube is formed from a plurality of biopolymers, wherein a first biopolymer of the plurality of biopolymers forms an inner surface of the main tube and a second biopolymer of the plurality of biopolymers is disposed circumferentially around the first biopolymer and forms an outer surface of the main tube, and wherein at least one of the first biopolymer or the second biopolymer is chitosan.
286. The ear tube of clause 260, wherein the ear tube comprises a first biopolymer forming at least a portion of a main tube of the ear tube and at least a portion of at least one flange, and a second biopolymer forming at least a portion of the main tube of the ear tube and at least a portion of at least one flange, and wherein at least one of the first biopolymer or the second biopolymer is chitosan.
287. The ear tube of clause 260, wherein an inner flange of the ear tube comprises chitosan and a main tube of the ear tube and outer flange comprise silk.
288. The ear tube of clause 260, wherein the ear tube comprises at least one biopolymer, the at least one biopolymer is silk fibroin.
289. The ear tube of clause 260, wherein the ear tube comprises at least one biopolymer, the at least one biopolymer is at least one of silk fibroin or chitosan polysaccharide.
290. The ear tube of clause 260, wherein the ear tube comprises silk fibroin and chitosan polysaccharide.
291. The ear tube of clause 260, wherein the ear tube comprises silk fibroin and alginate.
292. The ear tube of clause 260, wherein the ear tube comprises at least one biopolymer, the at least one biopolymer is an aqueous silk solution, an aqueous alginate solution, an aqueous fibrinogen solution, an aqueous chitosan solution, or an aqueous collagen solution.
293. The ear tube of clause 260, wherein the ear tube is formed via centrifugal molding.
294. The ear tube of clause 260, wherein the ear tube is formed via thermal molding and machining.
295. The ear tube of clause 260, wherein the ear tube is formed via centrifugal molding, thermal molding, and machining.
296. The ear tube of clause 260, wherein the ear tube comprises at least one biopolymer, the at least one biopolymer is assembled via an aqueous-based polymerization.
297. The ear tube of clause 260, wherein the ear tube comprises at least one additive.
298. The ear tube of clause 297, wherein the at least one additive is at least one of a bioactive molecule, a hydrophilic plasticizer, a hydrophobic plasticizer, a pigment, or a dye.
299. The ear tube of clause 298, wherein the bioactive molecule is an active pharmaceutical ingredient, an antibiotic, an anti-inflammatory agent, a steroid, a growth factor, or an enzyme.
300. The ear tube of clause 297, wherein the at least one additive is dip-coated onto the ear tube.
301. The ear tube of clause 300, wherein the dip-coated ear tube is subjected to water annealing.
302. The ear tube of clause 297, wherein the additive is dissolved in a biopolymer solution before dip-coating.
303. The ear tube of clause 297, wherein the ear tube is impregnated with the at least one additive.
304. The ear tube of clause 299, wherein the enzyme is at least one of a proteolytic enzyme, a serine protease, protease XIV, proteinase K. alpha-chymotrypsin, collagenase, MMP-1, MMP-2, or glucose oxidase.
305. The ear tube of clause 260, wherein the ear tube is designed to degrade on demand.
306. The ear tube of clause 305, wherein degradation commences when the ear tube is exposed to a solvent.
307. The ear tube of clause 306 wherein the solvent is at least one of a glucose solution, hydrogen peroxide, carbamide peroxide, an alcohol, a calcium chelator, or EDTA.
308. The ear tube of clause 260, wherein the ear tube is a tympanostomy tube.
309. The ear tube of clause 260, wherein the ear tube comprises a hollow cylindrical body having at least one flanged end.
310. The ear tube of clause 260, the ear tube comprising:
   a main tube, the main tube extending along a center axis defining a conduit for fluid communication, the main tube having a first end and a second end; and
   an outer flange extending radially away from the main tube on the first end.
311. The ear tube of clause 260, the ear tube comprising:
   a main tube, the main tube extending along a center axis defining a conduit for fluid communication, the main tube having a first end and a second end;
   an outer flange extending radially away from the main tube on a first end;
   an inner flange extending radially away from the main tube on a second end; and
   a frustoconical portion of the main tube extending longitudinally from at least one of the first end or the second end, the frustoconical portion extending beyond a width of the flange on the at least one of the first end or the second end.
312. An ear tube comprising chitosan assembled via an aqueous solution, the ear tube having at least one characteristic centrifugal property selected from the group consisting of:
   i) a uniform density across at least a portion of the ear tube;
   ii) at least a portion of the ear tube is optically nontransparent;
   iii) at least a portion of the ear tube is free of exterior layering;
   iv) at least a portion of the ear tube is free of interior layering;
   v) at least a portion of the ear tube is interior-void-free;
   vi) at least a portion of the ear tube is free of cylindrical extrusion gaps; and
   vii) at least a portion of the ear tube is free of layering.
313. An ear tube, comprising:
   a hollow cylindrical body having at least one flanged end, wherein the ear tube comprises chitosan, the ear tube having at least one characteristic centrifugal property selected from the group consisting of:
   i) a uniform density across at least a portion of the ear tube;
   ii) at least a portion of the ear tube is optically nontransparent;
   iii) at least a portion of the ear tube is free of exterior layering;
   iv) at least a portion of the ear tube is free of interior layering;
   v) at least a portion of the ear tube is interior-void-free;
   vi) at least a portion of the ear tube is free of cylindrical extrusion gaps; and
   vii) at least a portion of the ear tube is free of layering.
314. An ear tube, comprising:
   a main tube, the main tube extending along a center axis defining a conduit for fluid communication, the main tube having a first end and a second end; and
   an outer flange extending radially away from the main tube on the first end, wherein the ear tube comprises chitosan, the ear tube having at least one characteristic centrifugal property selected from the group consisting of:
   i) a uniform density across at least a portion of the ear tube;
   ii) at least a portion of the ear tube is optically nontransparent;
   iii) at least a portion of the ear tube is free of exterior layering;
   iv) at least a portion of the ear tube is free of interior layering;
   v) at least a portion of the ear tube is interior-void-free;
   vi) at least a portion of the ear tube is free of cylindrical extrusion gaps; and
   vii) at least a portion of the ear tube is free of layering.
315. An ear tube, comprising:
   a main tube, the main tube extending along a center axis defining a conduit for fluid communication, the main tube having a first end and a second end;
   an outer flange extending radially away from the main tube on a first end;
   an inner flange extending radially away from the main tube on a second end; and
   a frustoconical portion of the main tube extending longitudinally from at least one of the first end or the second end, the frustoconical portion extending beyond a width of the flange on the at least one of the first end or the second end, wherein the ear tube comprises chitosan, the ear tube having at least one characteristic centrifugal property selected from the group consisting of:
   i) a uniform density across at least a portion of the ear tube;
   ii) at least a portion of the ear tube is optically nontransparent;
   iii) at least a portion of the ear tube is free of exterior layering;
   iv) at least a portion of the ear tube is free of interior layering;
   v) at least a portion of the ear tube is interior-void-free;
   vi) at least a portion of the ear tube is free of cylindrical extrusion gaps; and
   vii) at least a portion of the ear tube is free of layering.
316. A method of forming an ear tube, comprising:
   depositing a chitosan solution into an ear tube mold;
   subjecting the ear tube mold and its contents to a first centrifugation;
   depositing a polymerization bath into the ear tube mold and subjecting the ear tube mold and its contents to a second centrifugation to polymerize the chitosan solution into a shape of an ear tube; and
   drying the ear tube.
317. The method of clause 316, wherein the ear tube dwells in the mold after the second centrifugation to complete polymerization before the drying.
318. The method of clause 316, further comprising subjecting the ear tube to at least one of cross-linking, dipcoating, covalent bonding, heat treatment, pressure treatment, water annealing, sterilization, surface modifications, chemical etching, chemical smoothing, or physical smoothing.
319. A method of degrading an ear tube, comprising:
   exposing an ear tube to a solvent,
wherein the ear tube comprises chitosan having at least one characteristic centrifugal property selected from the group consisting of:
   i) a uniform density across at least a portion of the ear tube;
   ii) at least a portion of the ear tube is optically nontransparent;
   iii) at least a portion of the ear tube is free of exterior layering;
   iv) at least a portion of the ear tube is free of interior layering;
   v) at least a portion of the ear tube is interior-void-free;
   vi) at least a portion of the ear tube is free of cylindrical extrusion gaps; and
   vii) at least a portion of the ear tube is free of layering.
320. The method of clause 319, wherein the solvent is at least one of a glucose solution, hydrogen peroxide, carbamide peroxide, an alcohol, a calcium chelator, or EDTA.
321. The method of clause 319, wherein exposing is over a period of at least one of minutes, hours, or days.

1.-35. (canceled)

36. A ventilation tube comprising at least one biopolymer, the ventilation tube having at least one characteristic centrifugal property selected from the group consisting of:
i) a uniform density across at least a portion of the ventilation tube;
ii) at least a portion of the ventilation tube is optically nontransparent;
iii) at least a portion of the ventilation tube is free of layering;
iv) at least a portion of the ventilation tube is interior-void-free; and
v) at least a portion of the ventilation tube is free of cylindrical extrusion gaps.

37. The ventilation tube of claim 36, comprising at least one of an outer flange or an inner flange.

38. The ventilation tube of claim 37, wherein at least one of the outer flange or inner flange comprises an insertion protuberance.

39. The ventilation tube of claim 36, wherein the ventilation tube has a uniform diameter throughout its length.

40. The ventilation tube of claim 36, wherein the ventilation tube has a uniform density throughout.

41. The ventilation tube of claim 36, wherein a main tube of the ventilation tube has an axial length at least several times its diameter.

42. The ventilation tube of claim 36, wherein the at least one biopolymer is assembled via an aqueous-based polymerization.

43. The ventilation tube of claim 36, wherein the ventilation tube comprises chitosan.

44. The ventilation tube of claim 36, wherein the ventilation tube is formed via centrifugal molding.

45. The ventilation tube of claim 36, wherein the ventilation tube is designed to dissolve upon administration of a solvent.

46. The ventilation tube of claim 36, wherein a main tube of the ventilation tube has an axial length at least several times its diameter.

47. The ventilation tube of claim 36, wherein the ventilation tube is an ear tube.

48. The ventilation tube of claim 36, wherein at least a portion of the ventilation tube is free of exterior or interior layering.

49. An ear tube, comprising a hollow cylindrical body formed of a uniform biopolymer material including chitosan, the hollow cylindrical body having a flanged end, wherein the ear tube is configured to dissolve upon administration of a solvent.

50. The ear tube of claim 49, wherein the flanged end has a uniform outer diameter.

51. The ear tube of claim 49, wherein the hollow cylindrical body and at least one flanged end comprises a main tube, the main tube extending along a center axis defining a conduit for fluid communication, the main tube having a first end and a second end;
an outer flange extending radially away from the main tube on a first end;
an inner flange extending radially away from the main tube on a second end; and
a frustoconical portion of the main tube extending longitudinally from at least one of the first end or the second end, the frustoconical portion extending beyond a width of the flange on the at least one of the first end or the second end.

52. The ear tube of claim 49, wherein the ear tube improves wound healing after dissolution.

53. The ear tube of claim 49, wherein the ear tube is formed via centrifugal molding.

54. The ear tube of claim 49, having at least one characteristic centrifugal property selected from the group consisting of:
i) a uniform density across at least a portion of the ear tube;
ii) at least a portion of the ear tube is optically nontransparent;
iii) at least a portion of the ear tube is free of exterior layering;
iv) at least a portion of the ear tube is free of interior layering;
v) at least a portion of the ear tube is interior-void-free; and
vi) at least a portion of the ear tube is free of cylindrical extrusion gaps.

55. A method of forming an ear tube, comprising:
depositing a chitosan solution into an ear tube mold;
subjecting the ear tube mold and its contents to a first centrifugation;
depositing a polymerization bath into the ear tube mold and subjecting the ear tube mold and its contents to a second centrifugation to polymerize the chitosan solution into a shape of an ear tube; and
drying the ear tube.

56. The method of claim 55, wherein the ear tube dwells in the ear tube mold after the second centrifugation to complete polymerization before the drying.

57. The method of claim 55, further comprising dissolving the ear tube upon administration of a solvent.

58. A ventilation tube that is made of chitosan and formed by a process comprising:
depositing a chitosan solution into a ventilation tube mold;
subjecting the ventilation tube mold and its contents to a first centrifugation;
depositing a polymerization bath into the ventilation tube mold and subjecting the ventilation tube mold and its contents to a second centrifugation to polymerize the chitosan solution into a shape of an ventilation tube; and
drying the ventilation tube.

59. The ventilation tube of claim 58 wherein the ventilation tube is an ear tube.
